(19) United States
(12) Patent Application Publication
(10) Pub. No.: US 2026/0117230 A1
(43) Pub. Date: Apr. 30, 2026

(54) INHIBITORY RNA NANOPARTICLES

(71) Applicant: Rutgers, The State University of New Jersey, New Brunswick, NJ (US)

(72) Inventors: Sarah Jean Nevins, New Brunswick, NJ (US); Ki-Bum Lee, New Brunswick, NJ (US); Thanapat Pongkulapa, New Brunswick, NJ (US); Sang Ah Yi, New Brunswick, NJ (US)

(21) Appl. No.: 19/373,606

(22) Filed: Oct. 29, 2025

Related U.S. Application Data
(60) Provisional application No. 63/713,541, filed on Oct. 29, 2024.

(51) Int. Cl. C12N 15/113; A61K 9/51; A61K 31/7105; C12N 9/00; C12N 9/12

(52) U.S. Cl. CPC C12N 15/113; A61K 9/5192; A61K 31/7105; C12N 9/1247; C12N 9/93; C12Y 207/07006; C12Y 605/01001; C12N 2330/30

(57) ABSTRACT

Provided herein are nanoparticles comprising up to two miRNAs or miRNA zippers and up to two siRNAs for inhibiting up to four target nucleic acids. Template DNA sequences and methods for preparing the nanoparticles are also described. Also provided are methods for using sequence specific nanoparticles to treat various disorders.

### CROSS-REFERENCE TO RELATED APPLICATIONS

This application claims the benefit of and priority to U.S. Provisional Application No. 63/713,541 filed on Oct. 29, 2024, the content of which is incorporated by reference in its entirety.

### STATEMENT REGARDING FEDERALLY SPONSORED RESEARCH

This invention was made with government support under 1803517 awarded by the National Science Foundation, NS132556, DC016612, NS130836, NS133003, EB005583, 01HL150852, and GM135141, awarded by the National Institutes of Health, and OC220235P1 awarded by the Department of Defense. The government has certain rights in the invention.

### REFERENCE TO A SEQUENCE LISTING

The content of the electronic sequence listing (18316100015.xml; Size: 78,827 bytes; and Date of Creation: Oct. 29, 2025) is herein incorporated by reference in its entirety.

### BACKGROUND

Therapeutic approaches that utilize ribonucleic acid (RNA) moieties have shown significant promise in addressing a broad spectrum of diseases and health conditions, from viral infections and genetic abnormalities to numerous cancer types. The pivotal role of RNA in a myriad of vital biological processes highlights its potential as a therapeutic agent. It is instrumental in gene expression and the synthesis of proteins. In contrast to DNA-based therapies, RNA's regulation of cellular processes is transient, thereby positioning it as a promising approach for drug development in clinical environments. Furthermore, DNA-based therapeutics may carry a risk of genotoxicity due to the possibility of off-target mutations in the genome, a concern not as prevalent in RNA-based approaches. In contrast, RNA-focused therapies can interact with biological processes by manipulating RNA, effectively addressing the fundamental issues present in various diseases. This approach enables the correction of molecular dysfunctions, offering a targeted therapeutic strategy for addressing various medical conditions. For instance, 17 RNA technologies have received clinical approval for therapeutic purposes. Besides these approved technologies, 222 distinct RNA technologies are undergoing clinical trials. These include antisense oligonucleotides (ASOs), microRNA (miRNA) mimics, messenger RNAs (mRNAs), small interfering RNAs (siRNAs), and aptamers.

While RNA interference (RNAi) therapeutics hold significant promise, the practical clinical application of these treatments hinges on the ability to deliver RNA to cells and tissues in a targeted and efficient manner. It is crucial to ensure that RNA drugs are accurately directed to their intended locations within the body to harness the full therapeutic potential of this promising technology. Various strategies, including viral vectors, liposomes, polymers, and nanoparticles (NPs), have been developed for delivering siRNA into cells. These methods utilize different materials for cell uptake and incorporate extended templates capable of carrying multiple siRNAs. This enhances the therapeutic potential and quantity of siRNA delivered. However, conventional RNAi technology with current delivery methods faces challenges, such as agent instability, limited therapeutic efficacy, and potential off-target effects. For example, small RNA silencing technologies such as miRNA inhibitors and siRNA have been correlated with off-target gene expression, which can be reduced but not eliminated through chemical modifications such as a 2′-O-methyl ribosyl substitution at position 2 from the 5′ end in the antisense strand. Modified nucleic acid strategies, such as locked nucleic acids (LNAs) connected between 2′-O and 4′-C, miRNA sponges, and miRNA masks, have also been developed to increase the specificity of these RNAi therapeutics. Furthermore, adeno-associated virus (AAV) and lipid nanoparticle (LNP) formulations have recently been used to transfect RNA therapeutics and vaccines, including for delivering COVID-19 vaccines, as they assist in RNA uptake into cells. However, a main issue with the AAV-mediated delivery system is off-target effects such as vaccine-induced immune thrombotic thrombocytopenia (VITT). It has been reported that the AAV shell and proteins can recruit platelets to surround foreign proteins, which induces abnormal blood clotting. In addition, polyethylene glycol (PEG), a common component of LNPs, is thought to cause severe allergic reactions, including anaphylaxis, upon vaccination.

The aforementioned challenges underscore the imperative need for advanced delivery methods to maximize the efficacy of RNA-based therapies. Consequently, there is an urgent need for an innovative platform capable of delivering small RNAs with high precision and minimal side effects aimed at either inhibiting or replicating natural nucleic acids.

### SUMMARY

In an aspect, provided herein is a composition comprising: a first DNA template comprising from 5′ to 3′: a first portion of a T7 promoter; a first double-strand template; a first single-strand template; a second double-strand template; and a second portion of a T7 promoter; and a second DNA template comprising from 5′ to 3′: the first portion of the T7 promoter; a second complement template that is reverse complementary to the second double-strand template; a second single-strand template; a first complement template that is reverse complementary to the first double-strand template; and the second portion of the T7 promoter; wherein the first single-strand template and the second single-strand template cannot hybridize; wherein the first double-strand template and the first complement template encode a first siRNA or a first random duplex; and wherein the second double-strand template and the second complement template encode a second siRNA or a second random duplex.

The first DNA template and the second DNA template may be between about 85 and about 95 nucleotides in length. In embodiments, the first DNA template and the second DNA template each are about 90 nucleotides in length.

Each of the first single-strand template and the second single-strand template may be between about 21 and about 23 nucleotides in length.

Each of the first single-strand template and the second single-strand template may encode a miRNA, a miRNA zipper, a miRNA mimic, a miRNA inhibitor, an antagomir, an aptamer, an antisense oligonucleotide (ASO), or a random sequence.

The first siRNA or random duplex and the second siRNA or random duplex each are between about 21 and about 23 base pairs in length.

In embodiments, the first siRNA or random duplex and the second siRNA or random duplex are different.

In another aspect, provided herein is a kit comprising the first DNA template and the second DNA template described herein, wherein the first DNA template and the second DNA template are in separate containers.

In another aspect, provided herein is an inhibitory RNA nanoparticle comprising a first RNA and a second RNA, wherein the first and second RNAs are partially hybridized to each other; the nanoparticle comprising from 5′ to 3′: a first double-stranded region, wherein the first RNA is hybridized to the second RNA; a bubble region, wherein the first RNA is not hybridized to the second RNA; and a second double-stranded region, wherein the first RNA is hybridized to the second RNA; wherein the first RNA is hybridized to the second RNA at the first double-stranded region and the second double-stranded region; wherein the first RNA and the second RNA cannot hybridize to each other; and wherein the first and second RNAs are between about 85 and about 95 nucleotides in length.

The first RNA and the second RNA may each be about 90 nucleotides in length. In the bubble region, the first RNA and the second RNA are between about 21 and about 23 nucleotides in length; and wherein the first double-stranded region and the second double-stranded region are each between about 21 and about 23 base pairs in length. In the bubble region, each of the first RNA and the second RNA may be a miRNA, a miRNA zipper, a miRNA mimic, a miRNA inhibitor, an antagomir, an aptamer, an ASO, or a random sequence. Each of the first double-stranded region and the second double-stranded region may be an siRNA or a random duplex.

In embodiments, the first double-stranded region and the second double-stranded region are different.

The nanoparticle may comprise a fluorescent label. In embodiments, the fluorescent label comprises 6-carboxyfluorescein (FAM).

The nanoparticle may be coated with a polymer or a lipid. An antibody may be conjugated to the nanoparticle.

In another aspect, provided herein is a method for preparing any of the inhibitory RNA nanoparticles described herein, the method comprising: providing a first 5′ phosphorylated linear DNA template encoding the first RNA and a second 5′ phosphorylated linear DNA template encoding the second RNA; and for each template: a) in a reaction mixture, heating the template with a primer DNA containing a T7 promoter sequence in a duplex buffer at about 95° C. for about 2 minutes; b) letting the reaction mixture cool to room temperature; c) adding T4 buffer comprising 10 mM MgCl2, about 2.5 mM MnCl2, and about 200 U μL⁻¹ T4 DNA ligase; d) incubating the reaction mixture for about 16° C. for about 18 hours; e) incubating the reaction mixture for about 65° C. for about 10 minutes; f) diluting the reaction mixture to about 1:10 produce a first circularized DNA template and a second circularized DNA template; g) adding EDTA to the reaction mixture to about 11 mM, and incubating the reaction at about 80° C. for about 30 minutes; h) adding an equimolar amount of the first and second circularized DNA template to a reaction mixture comprising T7 reaction buffer, ribonucleotides, and a T7 RNA polymerase; i) incubating the reaction mixture at about 37° C. for about 20 hours, thereby producing the inhibitory RNA nanoparticle; and j) washing the inhibitory RNA nanoparticle.

The method may further comprise k) incubating the inhibitory RNA nanoparticle with a fluorescently labeled oligonucleotide comprising a sequence complementary to a non-functional portion of the inhibitory RNA nanoparticle at a final concentration of about 10 μM at about 65° C. for about 10 minutes. Step f) may further comprise incubating the reacting mixture with at least one of exonuclease 1 and exonuclease 3 at about 37° C. for about 30 minutes.

### BRIEF DESCRIPTION OF THE DRAWINGS

FIGS. 1A-1C. MiR-133a zipper NP-mediated miRNA inhibition of miR-133a and induction of beige adipocytes. A) Schematic illustration of miR-133a zipper NPs encoding miRNA zipper for intracellular miRNA inhibition via complementary interaction. B) Fabrication process of miR-133a zipper NPs via RCT. C) MiR-133a zipper NPs inhibit miRNA-133a, resulting in upregulation of thermogenic genes (i.e., PRDM16, UCP1), hence increasing mitochondrial biogenesis and thermogenesis in adipocytes.

FIGS. 2A-2L. Synthesis and characterization of miR-133a zipper NPs. A) Schematic diagram of miR-133a zipper NP fabrication process via template circularization, followed by RCT. B) miR-133a (SEQ ID NO: 18) zipper designs used in this study, namely 11G11 (SEQ ID NO: 16) and 14G8 (SEQ ID NO: 17). Refer to FIG. 8 for full-size gel. C) Confirmation of template circularization after ligation. (M=100 bp ladder, +T4=ligation reaction in the presence of T4 ligase,−T4=ligation reaction in the absence of T4 ligase). Refer to FIG. 8 for full-size gel. D) Confirmation of exonuclease 1 and 3 digestion after template circularization (+Exo=Exonuclease 1 and 3). Refer to FIG. 8 for full-size gel. E) Confirmation of miR-133a zipper NP formation. Refer to FIG. 8 for full-size gel. F) Scanning electron microscope (SEM) image of miR-133a zipper NPs (scale bar=2 μm), inset: high magnification SEM image of miR-133a zipper NPs (scale bar=100 nm). G) Transmission electron microscope (TEM) image of miR-133a zipper NP (scale bar=100 nm), inset: higher magnification TEM image of RNA NP. H) Elemental composition of siRNA NPs I) NP concentration and size from NP Tracking Analysis (NTA). J) NTA video and K) AFM image of miR-133a zipper NPs (image is 10 μm by 8.33 μm). L) Schematic diagram of NP fabrication process.

FIGS. 3A-3C. Functional confirmation of released small RNAs from miR-133a zipper NP. A) Schematic illustration of miR-133a zipper NP system encoding both single-stranded RNA zipper complementary to miR-133a and double-stranded siRNA complementary to GFP mRNA for simultaneous gene inhibition. B) Images of native PAGE gels to test RNA binding to target. Refer to FIGS. 12, 14, and 16 for Full-size gels. C) Quantification of RNA binding to target and target sequence disappearance relative to the intensity of the fully unbound target from gels (n=3). Refer to FIGS. 12, 14, and 16 for Full-size gel triplicates.

FIGS. 4A-4G. Effects of miR-133a zipper NP delivery systems on thermogenic gene expression. A) Brightfield image of differentiated 3T3-LI adipocytes (scale bar=100 μm) B) Fluorescent images showing the cellular uptake of 11G11 miR-133a zipper NPs in a 3T3-L1 adipocyte (Green=fluorescein-labeled RNA NPs, Blue=nucleus staining by Hoechst 33342). C) The release of miRNA zipper from miR-133a zipper NPs in Dicer reactions. Refer to FIG. 20 for Full-size gels. D) GFP knockdown by miR-133a zipper NPs after 24 and 48 h of transfection (scale bar=50 μm). E) Quantification of GFP-positive 3T3-L1 adipocytes after treatment of miR-133a zipper. A one-way ANOVA with post-hoc Tukey test (n=2-3, *p<**p<0.01). F) Schematic illustration of thermogenic beige adipocyte generation from white adipocyte, induced by miR-133a zipper. G) Relative mRNA expression of key thermogenic genes upon treatment of adipocytes with different forms of miR-133a zipper. A one-way ANOVA with a post-hoc Tukey test (n=2-3, *p<0.05, **p<0.01, ****p<0.0001).

FIGS. 5A-5E. MiR-133a zipper NP induces thermogenic remodeling of adipocytes. A) Relative mRNA expression of key thermogenic genes after treatment of adipocytes with miR-133a zippers. A one-way ANOVA with a post-hoc Tukey test (n=2-3, *p<0.05, **p<0.01, ***p<0.001, ****p<0.0001). B) Western blot analysis of thermogenic factors after treatment of adipocytes with miR-133a zippers. Refer to FIG. 24 for full-size gel. C) Evaluation of mitochondrial biogenesis using MitoTracker Green staining. D) Fluorescence microscopic images of adipocytes stained for mitochondrial membrane (green) and nucleus (blue). E) Quantification of MitoTracker-stained cells, presented as relative intensity normalized to Hoechst signal. A one-way ANOVA with a post-hoc Tukey test (n=3, *p<0.05, **p<0.01, ***p<0.001, ****p<0.0001).

FIGS. 6A-6F. Reduction of adipose mass, and induction of thermogenesis and mitochondria biogenesis in adipose spheroids through miR-133a zipper NP. A) Schematic illustration of experimental workflow for evaluation of thermogenic remodeling in 3T3-LI adipose spheroids. B) Optical microscopic images and size measurement of adipose spheroids upon treatment with different forms of miR-133a zippers (image scale bar=100 μm). A two-way ANOVA with a post-hoc Tukey test (n=8, *p 0.05, **p 0.01, ***p 0.001, ****p 0.0001). C) Relative mRNA expression level of thermogenic genes from adipocyte spheroids treated with different forms of miR-133a zippers. A one-way ANOVA with post-hoc Tukey test (n=2-3, *p 0.05, **p 0.01, ***p 0.001, ****p 0.0001). D) Schematic illustration of experimental workflow for evaluation of thermogenic remodeling in adipose spheroids. E) Representative fluorescent images of adipocyte spheroids stained for mitochondrial membrane (using MitoTracker Green staining [Green]) and thermogenesis (using ERthermAC staining [Red]) after treatment with different miR-133a zippers (scale bar=200 μm). F) Quantification of fluorescent intensities of mitochondrial membrane and thermogenesis staining from treated adipose spheroids relative to the control. A one-way ANOVA with a post-hoc Tukey test (n=3, *p 0.05, **p 0.01, ***p 0.001, ****p 0.0001).

FIG. 7. Final RNA sequences formed from template A and B, as well as theoretical complementary binding shown between the two templates after transcription to RNA. SEQ ID NO: 26, top, SEQ ID NO: 27 bottom)

FIGS. 8A-8C. Full-size gel for A) FIG. 2C, B) FIGS. 2D, and C) FIG. 2E.

FIGS. 9A-9B. A) PDI and B) average size (nm) from DLS of three batches of 11G11 RNA nanoparticles for confirmation of batch-to-batch variation. A one-way ANOVA with a post-hoc Tukey test was run (n=3, *p<0.05, **p<0.01, ****p<0.0001).

FIG. 10. Elemental mapping data with FE-SEM with an EDX detector.

FIG. 11. LNA miRNA zipper design. A) Structure of locked nucleic acid (LNA). B) The designs of miR-133a zipper analogs. LNA=locked nucleic acid; NP=nanoparticle. SEQ ID NO: 16

FIG. 12. Full-size gels of 11G11 LNA, RNA for FIG. 3.

FIG. 13. Native PAGE gels and quantification of relative binding for 11G11 RNA, DNA and LNA sequences.

FIG. 14. Full-size gels of 14G8 LNA, RNA, and DNA for FIG. 3.

FIG. 15. Native PAGE gels and quantification of relative binding for 14G8 RNA, DNA, and LNA sequences.

FIG. 16. Full-size gels of complementary sequences for FIG. 3.

FIG. 17. Native PAGE gels and quantification of relative binding for 5′ to 3′ and 3′ to 5′ RNA sequences.

FIG. 18. 11A11 versus 11G11 binding efficiency outside of cells to confirm higher binding efficacy due to better gap in zipper sequence. A one-way ANOVA with a post-hoc Tukey test was run (n=3, *p<0.05, **p<0.01, ****p<0.0001).

FIG. 19. Full-size gels of 11A11 Gels for FIG. 18.

FIG. 20. Full-size gel of miRNA release from miR-133a zipper NPs in Dicer reactions.

FIG. 21A-21C. Stem-loop RT-qPCR for quantification of miR-133a zipper in RNA nanoparticle. A) Schematic of stem-loop RT-qPCR principle. B) Calibration Curve for determining amount of 11G11 RNA zipper. C) Sequences for stem-loop RT-qPCR. 11G11F: SEQ ID NO: 16; Complete: SEQ ID NO: 24; RT Primer SEQ ID NO: 20; 1st strand SEQ ID NO: 80; Primer F: SEQ ID NO: 21; 2nd strand SEQ ID NO: 81; Primer R SEQ ID NO: 22.

FIG. 22. Cell Viability data of RNA NP and LNA zipper in a concentration dependent manner. A one-way ANOVA with a post-hoc Tukey test was run (n=3, *p<0.05, **p<0.01, ****p<0.0001).

FIG. 23. RT-qPCR for quantification of PRDM16 and UCPI expression relative to GAPDH for A) 11G11 RNA zipper nanoparticle and B) LNA zipper delivered with lipofectamine. A two-way ANOVA with a post-hoc Tukey test (n=3 for PRDM16 nanoparticle; n=6 for all other conditions, *p<0.05, **p<0.01, ***p<0.001, ****p<0.0001).

FIG. 24. Unedited Western Blot Membranes for FIG. 5B.

FIG. 25. Changes in miR-133a levels with RNA zipper, LNA zipper, and the NP for both the 11G11 and 14G8 sequences.

FIG. 26. FAM-labeled miR-133a zipper NP uptake into adipose spheroids.

FIG. 27. Adipose spheroids treatment and size alterations over time. A) Comparison of nanoparticle, zipper in lipofectamine, and control Adipocyte Spheroid size on day 2 of nanoparticle delivery. B) Comparison of nanoparticle, zipper in lipofectamine, and control Adipocyte Spheroid size on day 4 of nanoparticle delivery. C) Comparison of nanoparticle, zipper in lipofectamine, and control Adipocyte Spheroid size on day 6 of nanoparticle delivery.

FIG. 28. Theoretical Volume of Spheroids treated with 11G11 or 14G8 miR-133a zipper and control conditions overtime and two-way ANOVA with a post-hoc Tukey test (n=8, *p<0.05, **p<0.01, ***p<0.001, ****p<0.0001).

FIG. 29. Ratio of spheroid volume after treatment with 11G11 or 14G8 miR-133a zipper over control condition volume overtime and two-way ANOVA with post-hoc Tukey test (n=8, *p<0.05, **p<0.01, ***p<0.001, ****p<0.0001).

FIG. 30. Schematic of annealing of small RNA zippers (e.g. miRNA zipper) to small RNA molecules (e.g. miRNA).

FIGS. 31A-31B. MicroRNA zipper NP-mediated miRNA inhibition of a miRNA. A) Schematic illustration of miRNA zipper NPs encoding miRNA zipper for intracellular miRNA inhibition via complementary interaction. B) Fabrication process of miRNA zipper NPs via RCT.

FIG. 32 illustrates the template sequence for an A_{2A}R nanoparticle and the resulting A_{2A}R siRNA. In descending order: Antisense SEQ ID NO: 31; template SEQ ID NO: 28; T7 Promoter SEQ ID NO: 1; Sense SEQ ID NO: 32; SEQ ID NO: 29; Anti-A2AR sense: SEQ ID NO: 30, antisense: 31; SEQ ID NO: 31; Convert to DNA bases: sense: SEQ ID NO: 32; antisense SEQ ID NO: antisense SEQ ID NO: 33 (SEQ ID NO: 32 and 33 in box and below box to right); Order from IDT: SEQ ID NO: 28 and 29.

FIGS. 33A-33L. Generation of nanoparticle-based siRNA-A_{2A}R silencing (siRNA-A_{2A}R NP). (A) Schematic diagram of A_{2A}R siRNA nanoparticle formation and coating. (B) DLS and (C) Zeta of RNA nanoparticles without and with chitosan coating. (D) TEM of chitosan coated RNA NP. (E) Cell uptake of chitosan-coated RNA NP over time. (F) Cell viability with RNA NP and chitosan-coated RNA NP. qRT-PCR of A_{2A}R expression relative to GAPDH after bare RNA nanoparticle delivery at (G) 24 hours and (H) 48 hours. (I) Western Blot of A_{2A}R expression and beta-actin housekeeping protein after bare RNA nanoparticle delivery. (J) Quantification of A_{2A}R protein levels relative to beta-actin from western blot. (K) qRT-PCR of relative A_{2A}R expression after delivery of bare A_{2A}R nanoparticles, PEI-coated A_{2A}R nanoparticles, and chitosan-coated A_{2A}R nanoparticles to mature hippocampal mouse neurons induced with A_{2A}R upregulation. (L) Chemical structure of A_{2A}R agonist NECA.

FIGS. 34A-34E. (A) schematic of RNA NP synthesis. (B) siRNA sequence targeting human A2AR (different from mouse sequence). (C) siRNA binding to sequence target ran with PAGE. (D) Nanoparticle formation and each step of synthesis is characterized with agarose gel electrophoresis. (E) Nanoparticle size characterization done with nanoparticle tracking analysis (NTA). Anti-A2AR sense SEQ ID NO: 82, antisense SEQ ID NO: 83; convert to DNA bases sense SEQ ID NO: 84; antisense SEQ ID NO: 85.

FIG. 35A-35D. (A) Schematic of antibody conjugation to RNA NP (B) Western Blot of RNA NPs, Chitosan-coated RNA NP, and antibody-conjugated RNA NP to show particle still functions with coating and antibody conjugation. (C) quantification of A2AR western blot relative to beta actin. (D) quantification of pCREB relative to beta actin which is downstream of A2AR.

FIG. 36. Transferrin antibody conjugation to RNA NP's led to higher uptake in endothelial cells than chitosan coated RNA NP alone.

FIGS. 37A-37C. (A) Schematic of cDNA formation and RNA nanoparticle formation. (B) Schematic of lipid coating of the RNA nanoparticle. (C) RT-qPCR data showing PDE2A RNA expression in the presence of bare nanoparticle and lipid-coated nanoparticle.

FIGS. 38A-38B. illustrate the template sequence for a (A) PDE2A2 nanoparticle and the resulting PDE2A2 siRNA, and a (Sequences in descending order antisense: SEQ ID NO: 37; template SEQ ID NO: 40; T7 promoter SEQ ID NO: 1; sense SEQ ID NO: 38; template SEQ ID NO: 35; anti-PEE2A2 sense SEQ ID NO: 36, antisense SEQ ID NO: 37; antisense SEQ ID NO: 39; SEQ ID NO: 40, SEQ ID NO: 41) (B) PDE2A nanoparticle and the resulting PDE2A siRNA. (Sequences in descending order antisense: SEQ ID NO: 43; template SEQ ID NO: 41; T7 SEQ ID NO: 1; sense SEQ ID NO: 42; template SEQ ID NO: 47; Anti-PDT2A SEQ ID NO: 42, SEQ ID NO: 43; IDT templates SEQ ID NO: 40, SEQ ID NO: 41.

FIG. 39 illustrates a nanoparticle that targets TLR4 and comprises two microRNAs (miR-21-5p and miR-21-3p).

FIG. 40 illustrates the template sequence for an anti-TLR4 and miR-21 nanoparticle. Sequences in descending order: Antisense SEQ ID NO: 49; template SEQ ID NO: 46; T7 SEQ ID NO: 1; sense SEQ ID NO: 50; template SEQ ID NO: 47; Anti-TLR4 sense: SEQ ID NO: 48; antisense SEQ ID NO: 49; convert to DNA bases sense SEQ ID NO: 48; antisense SEQ ID NO: 49; IDT primers SEQ ID NO: 46, SEQ ID NO: 47

FIGS. 41A-41B. (A) Cell viability after 24h-LPS treatment. RNA NPs with different concentrations. 1, 10, 20 μg/mL. (B) TLR4 and PTEN down regulation after simultaneous treatment of cells with 1 μg/mL of LPS and 20 μg/mL of RNA NP formulation.

FIG. 42 Ahnak nanoparticles. Antibodies are conjugated to the nanoparticles, as described in FIG. 35A.

FIG. 43 illustrates the template sequence for a miR-21 zipper and anti-VDAC 1 nanoparticle. In descending order antisense SEQ ID NO: 59; template SEQ ID NO: 56; T7 SEQ ID NO: 1; sense SEQ ID NO: SEQ ID NO: 58; template SEQ ID NO: 57; Anti-VDACI sense SEQ ID NO: 58; antisense SEQ ID NO: 59; convert to DNA bases SEQ ID NO: sense SEQ ID NO: 58; antisense SEQ ID NO: 59; Order from IDT SEQ ID NO: 56, SEQ ID NO: 57.

FIG. 44 illustrates the template sequence for a miR-21 zipper and anti-VDAC 2 nanoparticle. Sequences in descending order, antisense SEQ ID NO: template SEQ ID NO: 63; T7 SEQ ID NO: 1; sense SEQ ID NO: 65; template SEQ ID NO: 64; Anti-VDAC2 sense SEQ ID NO: 65; antisense SEQ ID NO: 66, miR-21 zipper SEQ ID NO: 69; Convert to DNA bases sense SEQ ID NO: 65; antisense SEQ ID NO: 66; order from IDT: SEQ ID NO: 56, SEQ ID NO: 57

### DETAILED DESCRIPTION

Described herein is a self-assembled particle of less than 400 nm in size, capable of simultaneously delivering multiple types of RNA, including siRNA, miRNA, and miRNA zipper. The particle template permits the generation of both single-stranded RNA and double-stranded RNA upon intracellular and extracellular processing by dicer and/or RNases. This nanoparticle is useful for treating a multitude of diseases including but not limited to diseases related to metabolic processing such as obesity, diabetes, pancreatic cancer, Tay-sachs disease (GM2 gangliosidosis), and Wilson's disease (hepatolenticular degeneration). Further applications include treatment of heart disease, other forms of cancer such as breast cancer, glioblastoma, and hepatocellular carcinoma, neurodegenerative diseases, and spinal cord injury.

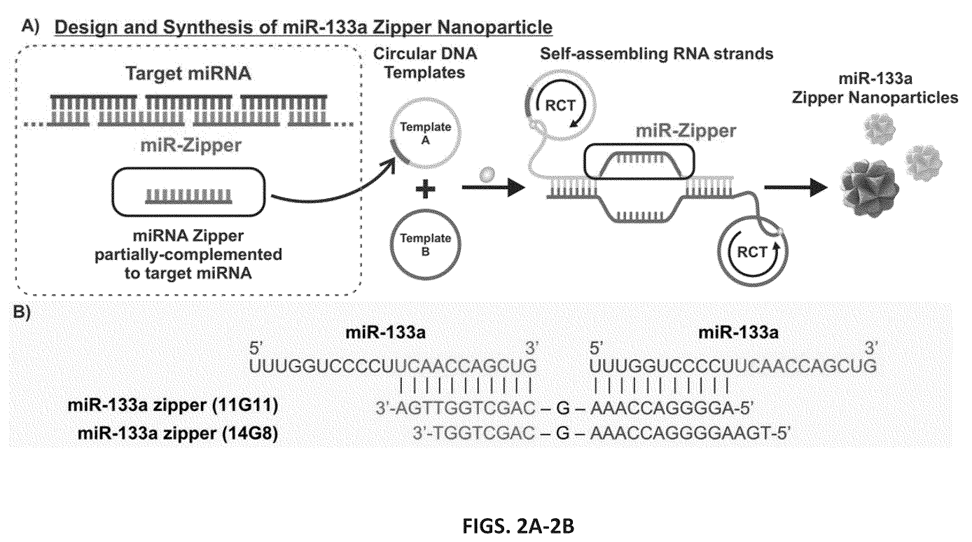

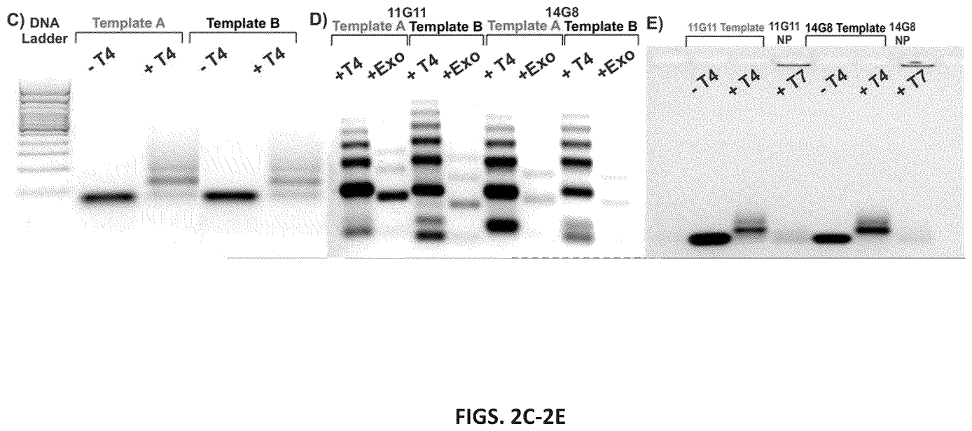

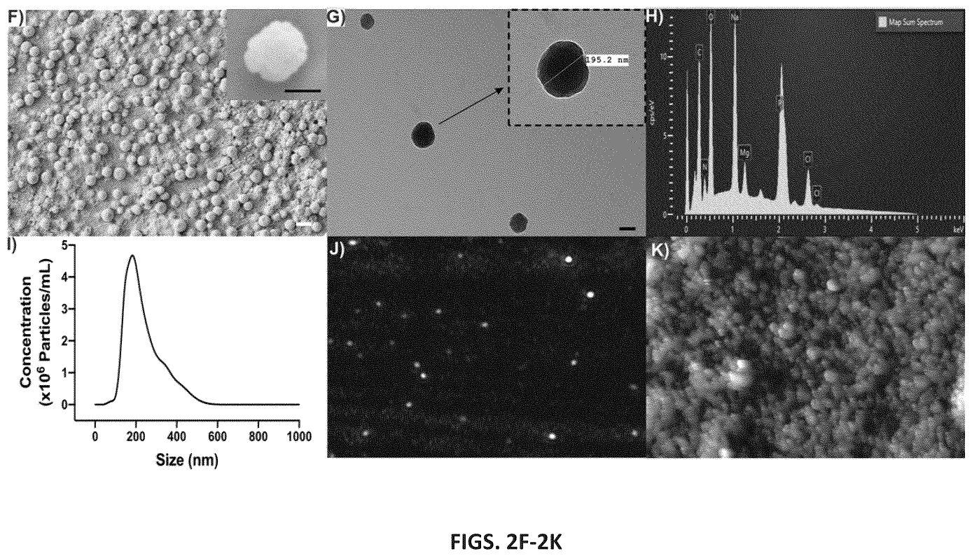

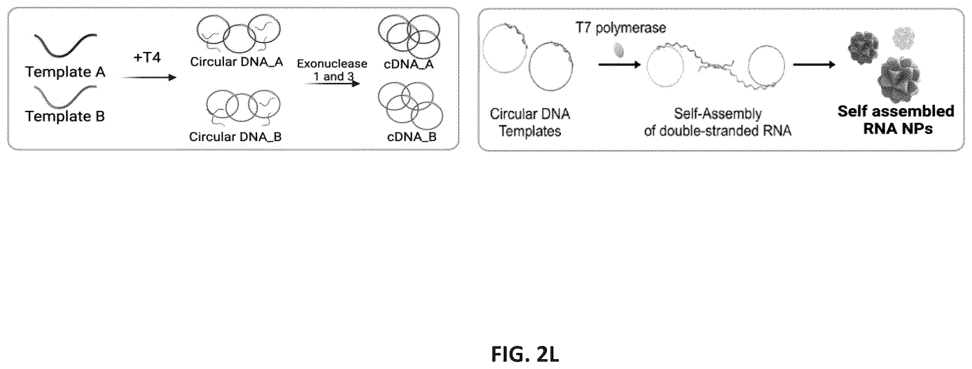

In a first aspect, provided herein is a composition comprising template sequences for preparing an inhibitory RNA nanoparticle, the template sequences comprising a first DNA template comprising from 5′ to 3′: a first portion of a T7 promoter; a first double-strand template; a first single-strand template; a second double-strand template; and a second portion of a T7 promoter; and a second DNA template comprising from 5′ to 3′: the first portion of the T7 promoter; a second complement template that is reverse complementary to the second double-strand template; a second single-strand template; a first complement template that is reverse complementary to the first double-strand template; and the second portion of the T7 promoter; wherein the first single-strand template and the second single-strand template cannot hybridize; wherein the first double-strand template and the first complement template encode a first siRNA, or a first random duplex; and wherein the second double-strand template and the second complement template encode a second siRNA or a second random duplex. The first and second DNA templates are designed such that when they are transcribed, the first-double strand template and first complement template are able to hybridize, and the second-double strand template and second complement template are able to hybridize, and the first single-strand template and second single-strand template are not able to hybridize with any sequences on either DNA template. This generates a structure comprising two RNA sequences having a bubble region of non-complementarity, flanked by two regions of complementarity, resulting in a nanoparticle as illustrated at FIG. 2A.

An inhibitory RNA nanoparticle is a nanoparticle comprised completely of RNA having both single-stranded regions and double-stranded regions, wherein at least of the regions encodes an inhibitory RNA. Examples of inhibitory RNAs are small RNA molecules (e.g. miRNAs, miRNA zippers, siRNAs, etc.) that bind to and inhibit the activity of other nucleic acid molecules through RNA interference.

In embodiments, the first DNA template comprises ATAGTGAGTCGTATTATN₁ (21-23) GN₂ (21-23) CN₃ (21-23) ATCCCT (SEQ ID NO: 90), wherein N₁ encodes a 21-23 nucleotide forward strand of an siRNA molecule or a random duplex, wherein N₂ encodes a 21-23 nucleotide single-stranded molecule that may be a miRNA, miRNA zipper, a miRNA mimic, a miRNA inhibitor, an antagomir, an aptamer, an antisense oligonucleotide (ASO), or a random sequence, and wherein N₃ encodes a 21-23 nucleotide forward strand of an siRNA molecule or a random duplex. N₁ and N₃ may be the same or different. The second DNA template may be reverse complementary to the first DNA template at N₁ and N₃.

The terms “polynucleotide,” “polynucleotide sequence,” “oligonucleotide,” “nucleic acid” and “nucleic acid sequence” refer to deoxyribonucleic acid (DNA), ribonucleic acid (RNA) and DNA/RNA hybrids. Polynucleotides may be single-stranded or double-stranded. Nucleic acids include, but are not limited to: genomic DNA (gDNA), complementary DNA (cDNA), synthetic RNA, synthetic DNA, or recombinant DNA.

The T7 promoter, having a consensus sequence of TAATACGACTCACTATAGGGA (SEQ ID NO: 1) is recognized by the T7 RNA polymerase for binding and initiation of transcription. The first portion and second portion of the T7 promoter are such that when the template sequences circularize, the T7 promoter is intact and recognized by the T7 polymerase.

As used herein, the terms “complementary” or “complementarity” are used in reference to “polynucleotides” and “oligonucleotides” related by the base-pairing rules as typically seen in the forward strand and reverse strand of a double-stranded DNA molecule. For example, the sequence “5′-C-A-G-T,” is complementary to the sequence “5′-G-T-C-A.” Complementarity can be “partial” or “total.” “Reverse complementary” refers to a sequence that is complementary to the reverse of another sequence. For example, the sequence “5′-C-A-G-T,” is reverse complementary to the sequence “5′-A-C-T-G.” “Partial” complementarity is where one or more nucleic acid bases is not matched according to the base pairing rules. “Total” or “complete” complementarity between nucleic acids is where each and every nucleic acid base is matched with another base under the base pairing rules.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26 (3/4): 227-259; and Owczarzy et al., 2008, Biochemistry, 47:5336-5353, which are incorporated herein by reference).

Each of the first and DNA templates are between about 85 and about 95 nucleotides in length, preferably about 90 nucleotides in length.

Each of the first single-strand template and the second single-strand template may be between about 21 and about 23 nucleotides in length. Each of the first single-strand template and the second single-strand template encodes a miRNA, a miRNA zipper, a miRNA mimic, a miRNA inhibitor, an antagomir, an aptamer, an antisense oligonucleotide (ASO), or a random sequence. The term “random sequence” as used herein refers to a single-stranded nucleic acid sequence that has no identified function or is not able to bind or hybridize with any molecule within a particular cell or organism.

In embodiments, the first single-strand template and the second single-strand template are different.

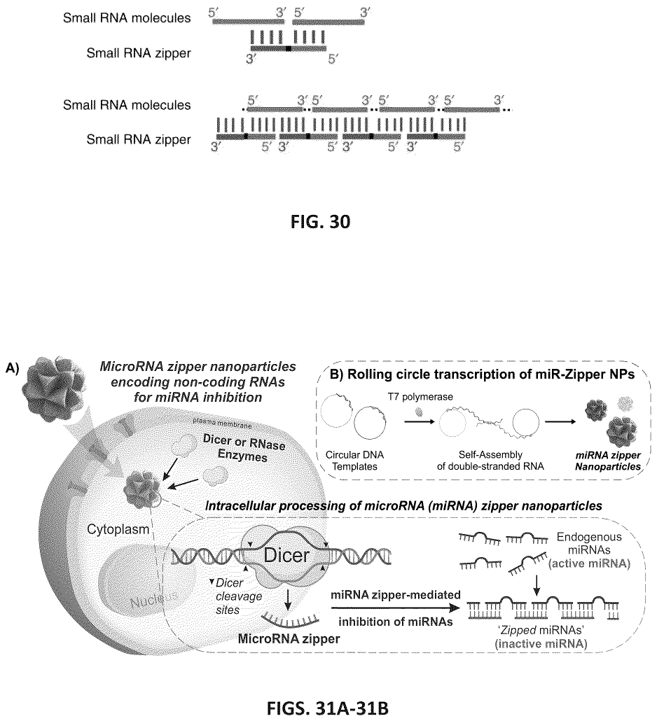

In embodiments, at least one of the first and second single-strand templates encode a miRNA zipper. The miRNA zipper may target miR-133a. The template encoding the miR-133a zipper may comprise SEQ ID NO: 24 or 25. A miRNA zipper is designed to bind to the 5′ half sequence of one miRNA molecule and the 3′ half sequence of another miRNA molecule through complementary hybridization, as illustrated at FIG. 30. The zipper binds the miRNAs end to end, forming a stable structure and blocking the function of the miRNA. The miRNA zipper may comprise a random nucleotide between a first portion that binds the 3′ half of the target miRNA and a second portion that binds the 5′ half of the target miRNA. In exemplary embodiments, the random nucleotide is a G or an A nucleotide. In preferred embodiments, the random nucleotide is a G.

At least one of the first double-strand template and first complement template, and second double-strand template and second complement template encode a small interfering RNA (siRNA) or a random duplex. A “random duplex” refers to a double-stranded nucleic acid sequence that has no identified function or is not able to bind or hybridize with any molecule within a particular cell or organism.

Each of the first siRNA or random duplex and the second siRNA or random duplex may be between about 21 and about 23 base pairs in length.

In embodiments the first siRNA or random duplex and the second siRNA or random duplex are different. In embodiments the first siRNA or random duplex and the second siRNA or random duplex are the same.

In embodiments, at least one of the siRNAs targets adenosine A_{2A} receptor (A_{2A}R). A_{2A}R is a G-protein coupled receptor critical for synaptic plasticity and memory, and has been identified as a promising therapeutic target for neurodegenerative diseases. In embodiments in which at least one of the siRNAs targets A2AR, the at least one of the double-strand template pairs (i.e. first double-strand template and first complement, and second double-strand template and second complement template) comprises SEQ ID NO: 32 and SEQ ID NO: 33.

In embodiments, at least one of the siRNAs targets a reporter protein, such as a fluorescent protein. In exemplary embodiments, the reporter protein is green fluorescent protein (GFP). For example, in embodiments, the first DNA template comprises SEQ ID NO: 2, 4; and the second DNA template comprises SEQ ID NO: 3, 5, wherein the templates encode a nanoparticle comprising a miR-133a zipper and GFP siRNA. In other embodiments, the first DNA template comprises SEQ ID NO: 28; and the second DNA template comprises SEQ ID NO: 29, wherein the templates encode a nanoparticle comprising an A_{2A}R siRNA and a GFP siRNA. Embodiments encoding siRNAs that target reporter proteins are useful for in vitro applications. Any reporter proteins known in the art may be used.

In embodiments, at least one of the siRNAs targets phosphodiesterase 2A (PDE2A) or PDE2A2. PDE2A is a cGMP-dependent 3′,5′-cyclic phosphodiesterase. PDE2A2 is a splice variant of PDE2A. In embodiments in which at least one of the siRNAs targets PDE2A/PDE2A2, the at least one of the double-strand template pairs (i.e. first double-strand template and first complement, and second double-strand template and second complement template) may comprise SEQ ID NO: 38 or 42, and SEQ ID NO: 39 or 43.

In an embodiment, at least one of the siRNAs targets toll-like receptor 4 (TLR4). TLR4 is a key activator of the innate immune response, and also plays a role in cancer growth, metastases, and treatment resistance of various malignancies. In embodiments in which at least one of the siRNAs targets TLR4, the at least one of the double-strand template pairs (i.e. first double-strand template and first complement, and second double-strand template and second complement template) may comprise SEQ ID NO: 48, and SEQ ID NO: 49.

In an embodiment, at least one of the single strand templates encodes microRNA miR-21. miR-21 is an oncogenic microRNA that plays a significant role in gene regulation, and is overexpressed in many types of cancer. In embodiments in which at least one of the single strand templates includes miR-21, the single strand template may comprise SEQ ID NO: 52 or 53.

In an embodiment, at least one of the single strand templates encodes a miR-21 zipper. In embodiments in which at least one of the single strand templates includes miR-21 zipper, the single strand template may comprise SEQ ID NO: 62.

In an embodiment, at least one of the siRNAs targets voltage-dependent anion-selective channel 1 (VDAC 1). VDAC 1 is the most abundant protein in the outer mitochondrial membrane, acting as a key channel that regulates the transport of ions and metabolites, such as ATP. In embodiments in which at least one of the siRNAs targets VDAC 1, the at least one of the double-strand template pairs (i.e. first double-strand template and first complement, and second double-strand template and second complement template) may comprise SEQ ID NO: 58, and SEQ ID NO: 59.

In an embodiment, at least one of the siRNAs targets voltage-dependent anion-selective channel 2 (VDAC 2). VDAC 2 is a protein that forms pores in the outer mitochondrial membrane, facilitating the transport of metabolites. In embodiments in which at least one of the siRNAs targets VDAC 2, the at least one of the double-strand template pairs (i.e. first double-strand template and first complement, and second double-strand template and second complement template) may comprise SEQ ID NO: 65, and SEQ ID NO: 66.

In an embodiment, at least one of the siRNAs targets tumor necrosis factor (TNFα). TNFα is a chemical messenger produced by the immune system that induces inflammation.

In an embodiment, at least one of the siRNAs targets interleukin-17 (IL-17). IL-17 is a group of pro-inflammatory cytokines that play a role in both the immune system's response to infection and the development of inflammatory diseases. In embodiments in which at least one of the siRNAs targets IL-17, the at least one of the double-strand template pairs (i.e. first double-strand template and first complement, and second double-strand template and second complement template) may comprise SEQ ID NO: 76, and SEQ ID NO: 77.

In an embodiment, at least one of the siRNAs targets glucose transporter type 1 (GLUT1). GLUTI is a protein that transports glucose into cells for energy.

In an embodiment, at least one of the siRNAs targets neuroblast differentiation-associated protein AHNAK (Ahnak). Ahnak is a protein shown to be essential for pseudopod protrusion and cell migration. In embodiments in which at least one of the siRNAs targets Ahnak, the at least one of the double-strand template pairs (i.e. first double-strand template and first complement, and second double-strand template and second complement template) may comprise SEQ ID NO: 86, and SEQ ID NO: 87.

In a second aspect, provided herein is a kit comprising any of the first DNA templates and second DNA templates described herein, wherein the templates are in separate containers.

In a third aspect, provided herein is an inhibitory RNA nanoparticle comprising a first RNA and a second RNA, wherein the first and second RNAs are partially hybridized to each other; the nanoparticle comprising from 5′ to 3′: a first double-stranded region, wherein the first RNA is hybridized to the second RNA; a bubble region, wherein the first RNA is not hybridized to the second RNA; and a second double-stranded region, wherein the first RNA is hybridized to the second RNA; wherein the first RNA is hybridized to the second RNA at the first double-stranded region and the second double-stranded region; wherein the first RNA and the second RNA cannot hybridize to each other; and wherein the first and second RNAs are between about 85 and about 95 nucleotides in length. The first RNA and the second RNA are each about 89 nucleotides in length. The inhibitory RNA nanoparticle may be encoded by any of the first and second DNA templates described herein.

In the bubble region, the first RNA and the second RNA may be between about 21 and about 23 nucleotides in length. In the bubble region, each of the first RNA and the second RNA is a miRNA, a miRNA zipper, a miRNA mimic, a miRNA inhibitor, an antagomir, an aptamer, or a single-stranded antisense oligonucleotide.

In embodiments, in the bubble region, the first RNA and the second RNA are different. In other embodiments, in the bubble region, the first RNA and the second RNA are the same.

In embodiments, in the bubble region, at least one of the first RNA and the second RNA is a miRNA zipper. The miRNA zipper may target miR-133a. The miRNA zipper may comprise SEQ ID NO: 15, 16, or 17. The miRNA zipper may target miR21. The miRNA zipper may comprise SEQ ID NO: 69.

In embodiments, in the bubble region, at least one of the first RNA and the second RNA is a miRNA. The miRNA may target miR-21. The miRNA may comprise SEQ ID NO: 54 or 55.

The first double-stranded region and the second double-stranded region may each be between about 21 and about 23 base pairs in length. Each of the first double-stranded region and the second double-stranded region is an siRNA or a random duplex.

In embodiments, at least one of the first double-stranded region and the second double-stranded region is an siRNA. In embodiments, the siRNA targets GFP. The first RNA may comprise SEQ ID NO: 26; and the second RNA may comprise SEQ ID NO: 27.

In embodiments, at least one of the siRNAs targets A2AR. The A_{2A}R siRNA may comprise a sense strand comprising SEQ ID NO: 30 and an antisense strand comprising SEQ ID NO: 31.

In embodiments, at least one of the siRNAs targets PDE2A/PDE2A2. The PDE2A2 siRNA may comprise a sense strand comprising SEQ ID NO: 36 and an antisense strand comprising SEQ ID NO: 37. The PDE2A siRNA may comprise a sense strand comprising SEQ ID NO: 44 and an antisense strand comprising SEQ ID NO: 45.

In embodiments, at least one of the siRNAs targets TLR4. The TLR4 siRNA may comprise a sense strand comprising SEQ ID NO: 50 and an antisense strand comprising SEQ ID NO: 51.

In embodiments, at least one of the siRNAs targets VDAC 1. The VDAC 1 siRNA may comprise a sense strand comprising SEQ ID NO: 60 and an antisense strand comprising SEQ ID NO: 61.

In embodiments, at least one of the siRNAs targets VDAC 2. The VDAC 2 siRNA may comprise a sense strand comprising SEQ ID NO: 67 and an antisense strand comprising SEQ ID NO: 68.

In embodiments, at least one of the siRNAs targets Ahnak. The Ahnak siRNA may comprise a sense strand comprising SEQ ID NO: 88 and an antisense strand comprising SEQ ID NO: 89.

The nanoparticle may comprise a fluorescent label. In exemplary embodiments, the fluorescent label comprises 6-carboxyfluorescein (FAM). Any fluorescent labels known in the art may be used such as GelRed, CybrGreen, Cy3, and Alexa Fluor dyes.

The nanoparticle may be coated with a polymer such as chitosan or a lipid. Other polymer coatings include polyethylenimine (PEI), cantonized gelatin, poly-l-lysine, cationic cellulose, cationic dextran, Poly(amidoamine), functionalized chitosan, DOPE, DOTAP, Cholesterol, phosphatidylcholine, mannose lipid, DSPE-PEG-maleimide.

In a fourth aspect, a method for preparing any of the inhibitory RNA nanoparticles described herein is provided, the method comprising: providing a first 5′ phosphorylated linear DNA template encoding the first RNA and a second 5′ phosphorylated linear DNA template encoding the second RNA; and for each template: a) in a reaction mixture, heating the template with a primer DNA containing a T7 promoter sequence (10 μM) in a duplex buffer (30 mM HEPES, pH 7.5, 100 mM potassium acetate) at between about 25 and about 37° C. for about 1 minute to 2 hours; b) letting the reaction mixture cool down to room temperature; c) adding T4 buffer (50 mM Tris-HCl, 10 mM MgCl₂, 1 mM ATP, 10 mM DTT, pH 7.5), about 2.5 mM MnCl2, and about 200 U μL⁻¹ T4 DNA ligase; d) incubating the reaction mixture for between about 15 and about 20° C. for 16 to 20 hours; e) incubating the reaction mixture at about 60 to about 70° C. for between about 5 and about 15 minutes; f) diluting the reaction mixture to about 1:10 with 10× Neb buffer 1 to produce a first circularized DNA template and a second circularized DNA template; g) adding EDTA to the reaction mixture to about 11 mM, and incubating the reaction at between about 70 and 80° C. for between about 15 and about 30 minutes; h) adding an equimolar amount of the first circularized DNA template and the second circularized DNA template to a reaction mixture (about 200 μL) comprising T7 reaction buffer, ribonucleotides, and a T7 RNA polymerase; i) incubating the reaction mixture at between about 35 and about 40° C. for between about 19 and 24 hours, thereby producing the inhibitory RNA nanoparticle; j) washing the inhibitory RNA nanoparticle. The nanoparticle may be washed by centrifugation and resuspension with nuclease-free at least once. The nanoparticle may be stored at about 4° C.

The 5′ phosphorylated linear DNAs may be PAGE purified and provided at about 10 μM.

The method may further comprise fluorescently labeling the nanoparticle, by k) incubating the inhibitory RNA nanoparticle with a fluorescently labeled oligonucleotide comprising a sequence complementary to a non-functional portion of the inhibitory RNA nanoparticle at a final concentration of between about 0.5 and about 2 μg/uL at between about 20° C. and about 37° C. for between about 1 and about 30 minutes.

The method may further comprise at step f), incubating the reacting mixture with exonuclease 1 and exonuclease 3 at between about 35 and 40° C. for between about 20 and about 40 minutes.

Step a) may be performed at about 95° C. for about 2 minutes. Step d) may be performed at about 16° C. for about 18 hours. Step e) may be performed at about 65° C. for about 10 minutes. Step f) may be performed at about 37° C. for about 30 minutes. Step g) may be performed at about 80° C. for about 30 minutes. Step i) may be performed at about 37° C. for about 21 hours. The microparticle may then be stored at between about 0-4° C.

In embodiments in which a fluorescent label is added step k), may be performed at about 65° C. for about 10 minutes.

In embodiments, the method may further comprise conjugating an antibody to the nanoparticle. The antibodies may be conjugated by the methods described below, or any other appropriate method known in the art.

The terms “antibody” and “antibody molecule” are used herein interchangeably and refer to immunoglobulin molecules or other molecules which comprise an antigen binding domain. Antibodies include whole antibodies (e.g., IgG, IgA, IgE, IgM, or IgD), monoclonal antibodies, chimeric antibodies, humanized antibodies, and antibody fragments, including single chain variable fragments (ScFv), single domain antibodies, and antigen-binding fragments, genetically engineered antibodies, among others, as long as the characteristic properties (e.g. ability to bind to the protein of interest or variant) are retained.

The term antibody includes “antibody fragments” or “antibody-derived fragments” and “antigen binding fragments” which comprise an antigen binding domain and displays antigen binding function, for example, Fab, Fab′, F(ab′) 2, scFv, Fv, dsFv, ds-scFv, Fd, mini bodies, monobodies, and multimers thereof and bispecific antibody fragments. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), (see for instance Bird et al., Science 242, 423-426 (1988) and Huston et al., PNAS USA 85, 5879-5883 (1988)). Such single chain antibodies are encompassed within the term antibody unless otherwise noted or clearly indicated by context.

In a fifth aspect, provided herein is a method of treating a subject in need thereof with any of the inhibitory RNA nanoparticles described herein. In embodiments, the method comprises inhibiting miR-133a in a subject in need thereof by administering to the subject a therapeutically effective amount of the inhibitory RNA nanoparticle comprising a miR-133a zipper described herein. The subject may suffer from obesity or a metabolic disorder related to obesity.

In embodiments, the method comprises inhibiting A_{2A}R in a subject in need thereof by administering to the subject a therapeutically effective amount of the inhibitory RNA nanoparticle comprising an A_{2A}R siRNA described herein. The nanoparticle may be coated with chitosan. The subject may have or be at risk of developing a neurological disorder or a cancer. In embodiments, the method comprises inhibiting PDE2A in a subject in need thereof by administering to the subject a therapeutically effective amount of the inhibitory RNA nanoparticle comprising a PDE2A siRNA described herein.

Chitosan coating may comprise stirring about 100 μL of 0.2% low-molecular weight chitosan and about 100 μg nanoparticles in nuclease free water with 0.1% acetic acid for about 30 minutes to 4 hours at room temperature, then centrifuging the mixture at about 12,000 rpm for about 30 minutes to purify the nanoparticles. In preferred embodiments, the chitosan and nanoparticles are stirred for about 1 hour.

Lipid coating may comprise adding phosphatidylcholine and cholesterol to chloroform in a round bottom flax, removing the solvents under rotary evaporation forms a thin film, adding purified RNA to the thin film, and sonicating for about 30 minutes to produce liposomes of ˜200 nm. The encapsulated nanoparticles may then be purified by centrifuging them at about 10,000 rpm for about 10 minutes. The encapsulated nanoparticles may then be doped with DSPE-PEG-maleimide lipid at a about 1% molar ratio through post-insertion.

In exemplary embodiments, the phosphatidylcholine and cholesterol are added at a ratio of 7:3. However, other ratios may be used.

The terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the term “administering” refers to dispensing, delivering or applying the substance to the subject. In terms of the pharmaceutical composition, therapeutic agent, or nanocarrier comprising the therapeutic agent, the term “administering” is intended to refer to delivering or applying it to the subject by any suitable route for delivery of the therapeutic agent to the desired location within the subject.

The inhibitory RNA nanoparticles disclosed herein may also be incorporated into pharmaceutical compositions. The disclosed nanocarriers or pharmaceutical compositions comprising the same may be used in the methods described herein. The pharmaceutical compositions may further comprise one or more pharmaceutically acceptable excipients. The pharmaceutically acceptable excipients will be dependent on the mode of administration to be used. Suitable modes of administration include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseous, periocular, intratumoral, intracerebral, and intracerebroventricular administration.

Any suitable dose of the disclosed inhibitory RNA nanoparticles or pharmaceutical compositions may be used. Suitable doses will depend on the intended therapeutic effect, body weight of the individual, age of the individual, etc.

The term “subject” or “patient” are used herein interchangeably to refer to a mammal, preferably a human, to be treated by the methods and compositions described herein. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Preferably, the subject is a human. A “subject in need thereof” refers to a subject who would benefit from treatment with a nanoparticle described herein. The subject may have a disease or disorder in which a nucleic acid targeted by an inhibitor RNA in the nanoparticle is overexpressed or causes or aggravates a disease or disorder.

In a sixth aspect, provided herein is a method of inhibiting the expression or activity of a target nucleic acid in a cell, the method comprising contacting the cell with any of the inhibitory RNA nanoparticles described herein. The inhibitory RNA nanoparticle may comprise a fluorescent label, such as FAM. The inhibitory RNA nanoparticle may comprise an siRNA or other inhibitory molecule that inhibits a fluorescent protein. The cell may be any cell type, such as an adipocyte or a neural cell. Contacting the cell may comprise transfecting the cell with a lipid carrier containing the nanoparticle.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

### Example 1. Developing MiR-133a Zipper Nanoparticles for Targeted Enhancement

of Thermogenic Adipocyte Generation

### INTRODUCTION

RNA-based NP technologies show promise in surmounting the limitations of traditional RNA therapeutics, including low loading capacity, brief molecular half-life, and unintended off-target effects. [5,17-19] Moreover, they can also be functionalized by targeting ligands for directed delivery to specific cells or tissues. This makes NPs an ideal vehicle for delivering RNA therapeutics. Initial self-assembled RNA particle design by Lee et al. showed the potential of RNA

self-assembly into NPs. [20] Subsequently, RNA NPs have been confined to smaller sizes and had template modification to deliver multiple siRNA at once. [18,21] In short, RNA-based NP technologies have the potential to revolutionize the treatment of many diseases, such as cancer, genetic disorders, and infectious diseases. NP-based methods offer the potential to deliver various types of RNA biomolecules. An example is the miRNA zipper, developed by Meng et al., which

has demonstrated enhanced efficacy in miRNA silencing. The zipper system inhibits the action of endogenous oligonucleotides through end-to-end connection to form a zipper structure with the target, therefore silencing its detrimental effect. [22] This system potentially advances current RNAi technology by creating miRNA zipper NPs for the enhanced silencing of miRNAs critical to disease onset. For example, utilizing the miRNA zipper design has great potential for inhibiting miR-133a, a critical miRNA target for controlling adipocyte cell remodeling through regulation of the thermogenic gene expression pathway. [23,24] These advancements might revolutionize the development of RNAi-based therapeutics for various diseases.

One area of particular interest is the study and manipulation of adipocytes, which play a crucial role in energy balance and metabolism. Adipocytes store surplus energy as lipid droplets, thereby preventing fat accumulation in other tissues and protecting them from lipotoxicity. Understanding and potentially altering the function of these cells through RNAi could provide novel insights into energy expenditure, involving processes such as the activation of brown fat or the transformation of white fat into brown-like fat. This approach is especially relevant given the distinct ways in which different types of adipocytes, such as white and brown adipocytes, store energy in lipid droplets. Brown adipocytes have a larger surface area, facilitating easier energy utilization in the form of lipids. Additionally, it is worth noting that heat generation can induce energy expenditure. [27] For this purpose, miR-133a is a critical barrier to adipocyte browning by directly targeting the 3′ UTR on Prdm16, a key transcription factor for thermogenic gene expression. Prdm16 directs Myf5-positive progenitor cells with bidirectional cell fates, muscle cells, and brown adipocytes toward a brown fat lineage. [28] There are two types of brown fat cells—innate brown adipocytes and inducible brown-like fat cells or beige adipocytes. Brown adipose differentiation is promoted through ectopic PRDM16 expression in myoblasts, activating brown fat-selective transcriptional programing. [28,29] More importantly, forced PRDM16 expression in white fat depots triggers the formation of beige adipocytes with increased thermogenic gene expression, including UCP1. [30,31] UCP1-enriched adipocytes can consume stored lipids through non-shivering heat generation, [32] so the phenotypic changes promoted in inducible beige adipocytes through thermogenic gene expression would be desirable for effectively treating obesity and related metabolic complications.

Hence, miR-133a inhibition or depletion, which induces PRDM16, has been investigated as a browning strategy. Preventing PRDM16 suppression through the transfection of anti-miR-133 [24] or knockout of miR-133a. [33] resulted in adipocyte browning of subcutaneous adipose tissues. Our earlier study identified that reducing miR-133a levels with a small molecule promotes the browning of white adipose tissues and exhibits anti-obesity effects. [34] Excess lipid accumulation in vivo causes the expansion of adipose tissues through an increase in either adipocyte number (hyperplasia) or size (hypertrophy). [35,36] Hyperplasia occurs with angiogenesis and prevents the onset of insulin resistance, while hypertrophic expansion of adipocytes results in inflammation, fibrosis, and hypoxia and is linked to a decrease in angiogenesis. [35-37] Furthermore, hypertrophic adipocyte remodeling in obesity increases the likelihood of developing metabolic disorders by altering the adipokine secretome and inducing insulin resistance. Adipocyte remodeling into thermogenic adipocytes can help circumvent the harmful effects of excessive body fat accumulation to improve metabolic status. [38] As a result, the inhibition of miR-133a holds great potential in promoting adipocyte remodeling and serving as an essential therapeutic strategy for obesity treatment.

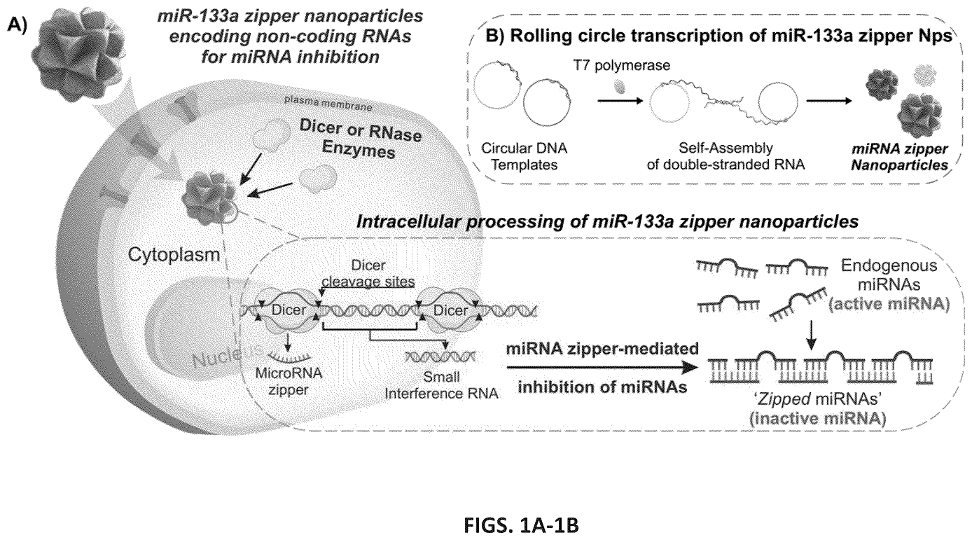

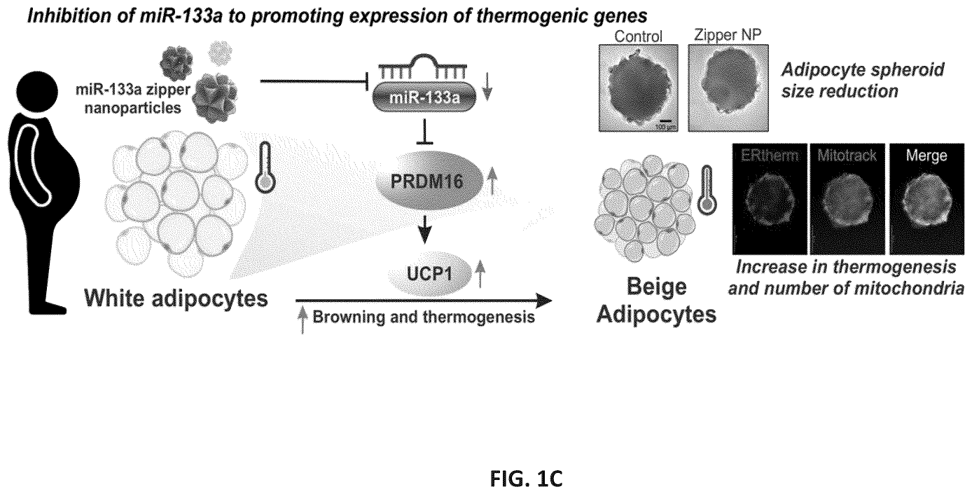

To advance miRNA-based therapies and address critical challenges related to obesity treatment, herein we developed a novel hybrid miR-133a zipper NP system that induces the browning of white adipocytes effectively and selectively, as depicted in FIG. 1A. This innovative, bio-inspired NP design can suppress specific endogenous or targeted miRNAs through a miRNA zipper mechanism. Additionally, it can deliver siRNA together with miRNA, thus enhancing the therapeutic potential of the system (FIG. 1A).

In the context of T7 RNA polymerase activity, we used rolling circle transcription (RCT) with circular DNA (cDNA) templates. These templates contained the miR-133a zipper and GFP siRNA sequences, which formed self-assembling NPs (FIG. 1B). We confirmed that these NPs were uptaken by cells and processed by intracellular dicer to simultaneously release miR-133a zippers to inhibit miR-133a and siRNA targeting GFP for gene silencing. We assessed the significant effects of the miR-133a zipper-releasing NPs on thermogenic gene expression and mitochondrial biogenesis in adipocytes. The miR-133a zipper showed better inhibition of miR-133a compared to other inhibitory sequences, as evidenced by its stronger binding capacity and therapeutic effectiveness. Moreover, the treatment of 3D adipose spheroids with the miR-133a zipper NP system resulted in a decrease in adipose tissue size and an increase in thermogenesis, suggesting the potential of the system for the treatment of obesity (FIG. 1C). Moreover, we confirmed that the template strand was cleaved by dicer to release multiple RNA sequences and that the NP was taken up by cells, which was achieved by incorporating a siRNA sequence for GFP-silencing into the miR-133a zipper NP. Overall, our innovative hybrid miR-133a zipper NP system not only enhances the efficiency of the miR zipper through NP-based delivery, but also demonstrates the possibility of delivering multimodal RNAi therapeutics altogether, highlighting its wide range of potential applications in biomedicine.

### Results and Discussion

Design, Synthesis, and Characterization of miRNA Zipper NPs

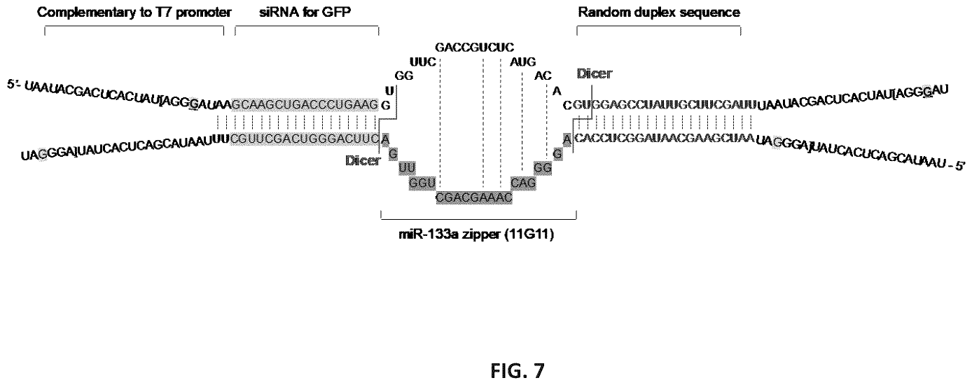

DNA strands were carefully designed to enable the creation of DNA templates capable of transcription and self-assembly into a repeated bubble-like RNA secondary structure. This specific structure is essential for the successful formation of the NPs. To this end, two linear single-stranded DNA templates encompassing the sequence for the miR-133a zipper and either the sense or antisense GFP siRNA underwent T4 ligation for cDNA template formation, followed by in vitro transcription using T7 polymerase for miR-133a zipper NP fabrication (FIGS. 2A and 2L). As the dicer-induced cleavage of long double-stranded RNAs preferentially occurs within 21-23 nucleotides from the overhang, the complimentary duplex region had 21 nucleotides for releasing miR-133a zipper from the NP without junk nucleotides (FIG. 7). Based on an earlier study, the miR-133a zipper sequences were designed to have the 5′ and 3′ regions of miR-133a, in which the two sides of the zipper are reversely linked with one gap nucleotide in the structure. Therefore, the 5′ and 3′ sides of the miR-133a zipper can bind to part of the miR-133a target sequence through a complementary interaction (FIG. 2B).

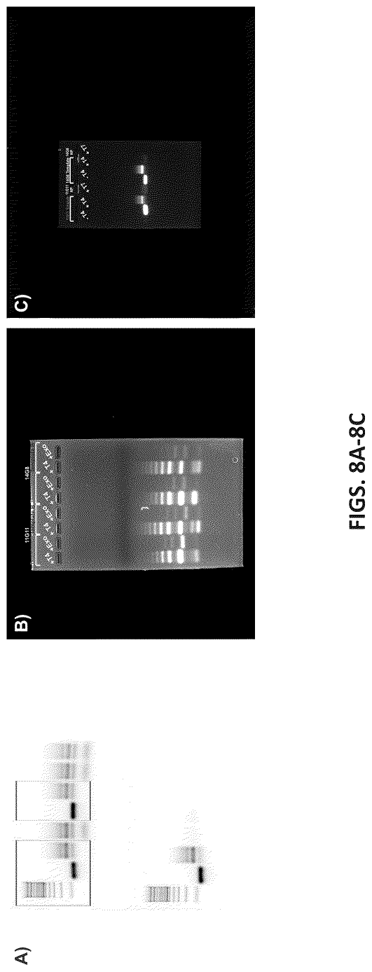

We generated two miR-133a zipper sequence designs with varying 5′ and 3′ arm lengths (11G11, same length of arms, 14G8, longer 5′ arm) to determine the most effective zipper sequence (FIG. 2B). The designed miR-133a zipper sequence includes a G base gap between the sequences binding each portion of the miRNA because of the need for a base gap for efficient binding, as well as the presence of an AAA repeat in miR-133a sequence which could prevent proper binding with an A base gap. Following the process of cDNA formation, the transformation from a linear to a circular conformation of the DNA structure resulted in a decrease in migration rate during gel electrophoresis. This is because cDNA moves slower than linear DNA in native PAGE, primarily due to its bulky shape. Therefore, a slower migration of cDNA compared to linear DNA confirmed the successful formation of the circular template (FIG. 2C). Exonucleases 1 and 3 were used to degrade improperly formed cDNA, as a more uniform template led to better NP formation, ensuring that the starting template was fully formed cDNA (FIGS. 2D and 8). The cDNA templates formed through T4 ligation were subsequently utilized for RCT with T7 polymerase to generate miR-133a zipper NPs (FIGS. 2E and 8).

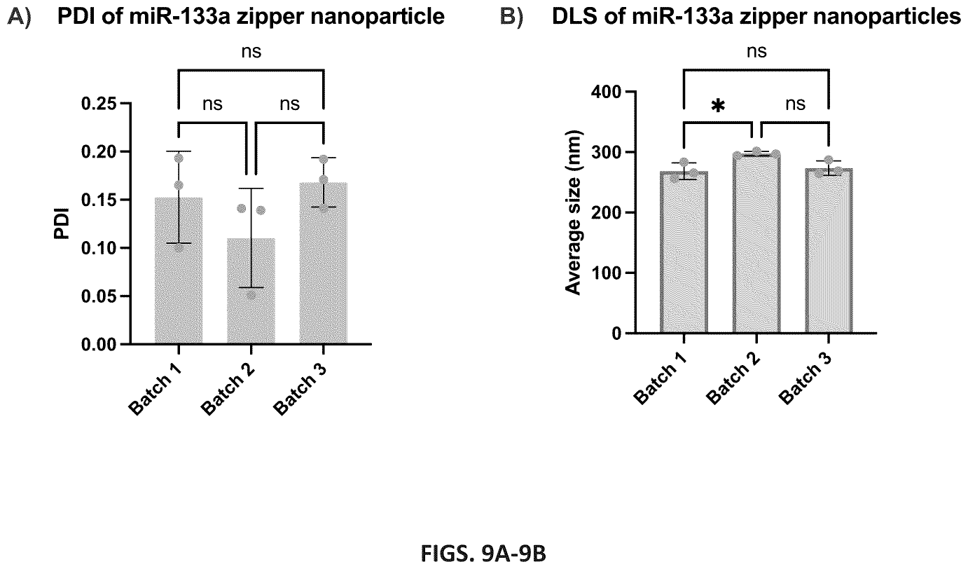

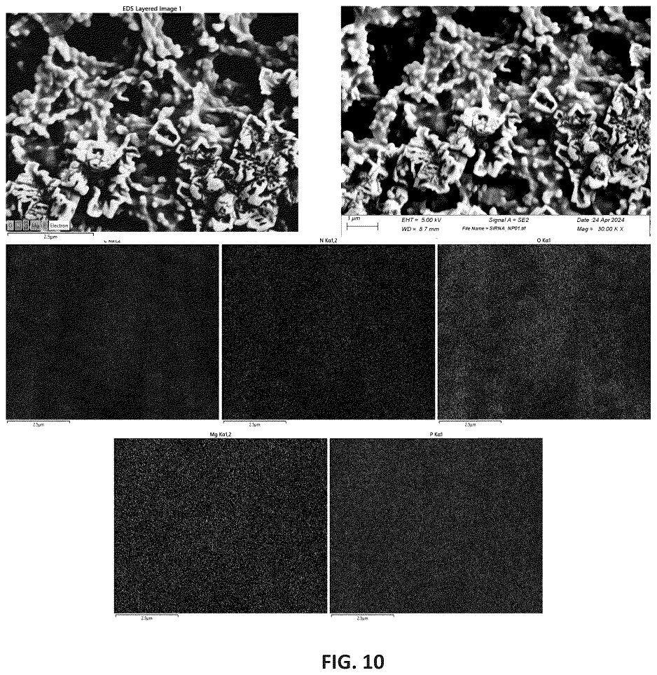

The size and surface morphology of the as-synthesized miR-133a zipper NPs was confirmed by scanning electron microscope (SEM) and transmission electron microscope (TEM). The SEM images revealed the spherical shape of the particles with the surface roughness, which is consistent with RNA NPs observed from literature using similar synthetic methods (FIG. 2F). [20,41-44] The SEM and TEM images showed the NPs had a size ≈200 nm (FIGS. 2F,G). Batch-to-batch variation was tested with DLS, and no significant size difference was observed across the three batches. The average hydrodynamic size of all three batches was 79.73 nm, with a standard deviation of 16.37 nm. (FIG. 9). Furthermore, the NPs have a low average polydispersity index across three batches at 0.144 with a standard deviation of 0.045 (FIG. 9). The elemental composition of the NPs was analyzed using energy-dispersive X-ray spectroscopy (EDS), which verified the presence of carbon, oxygen, phosphorus, and nitrogen, as well as a trace amount of Mg2⁺ ions, confirming the successful incorporation of the designed components into the NP structure. Mg2⁺ ions have been shown to help limit siRNA degradation by stabilizing RNA tertiary structure (FIGS. 2H and 10). [45,46] MgCl₂ is a reaction buffer component used for cDNA formation and NP synthesis, and a small amount was incorporated into the NP during synthesis. Furthermore, Mg2⁺ has been shown to promote cell entry and endosomal escape through Mg2⁺ ion interactions with the lipid bilayer. [17,45] Nanoparticle Tracking Analysis (NTA) assay confirmed the homogeneity of miR-133a zipper NPs (FIGS. 2I,J). The particles were monodispersed, with the majority population at a size of 174.4 nm and a mean size of 236.9 nm. Atomic force microscopy (AFM) was also used to elucidate the morphology and surface of the NPs (FIG. 2K), compared to previously reported miRNA-inhibiting technologies. Moreover, we inserted the zipper seq.

Optimization of miRNA-133a Zippers for Effective Inhibition

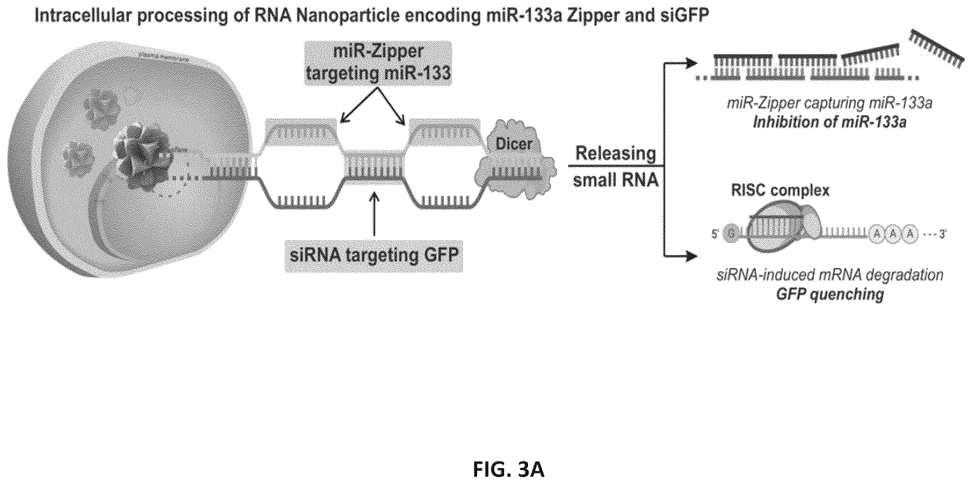

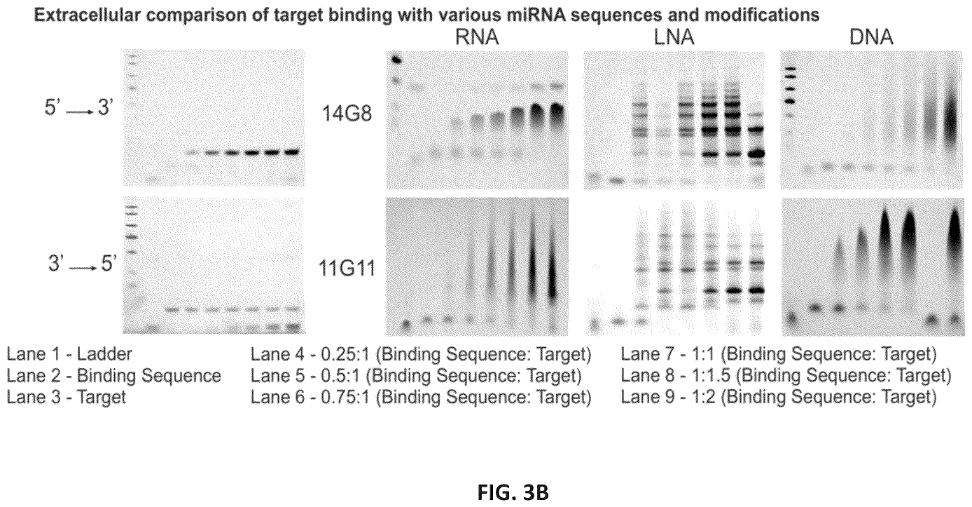

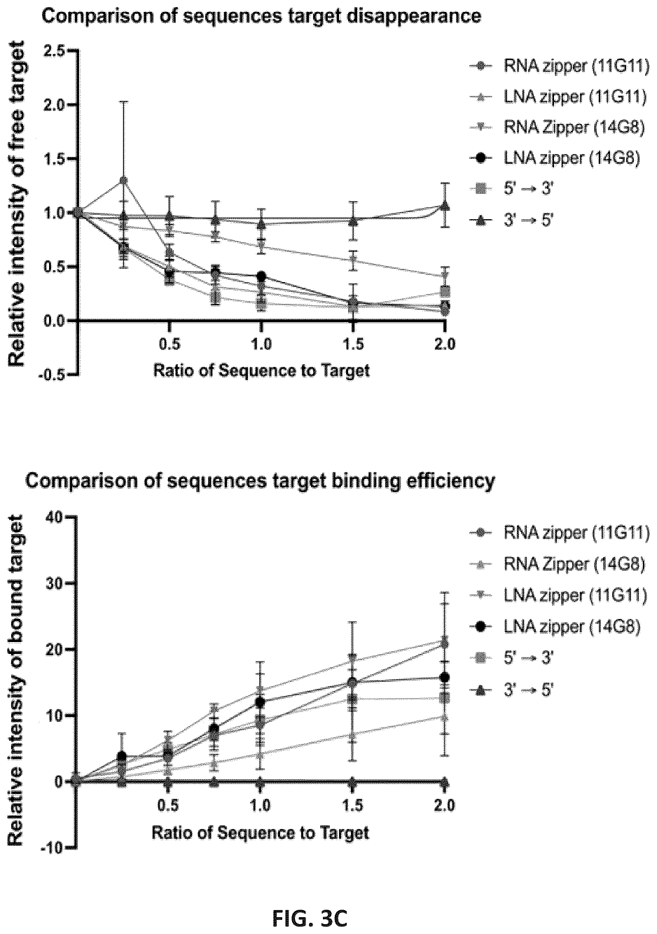

RNAi-based therapy has become a critical therapeutic approach, including a variety of forms allowing for diverse treatment options. RNAi therapeutics aims to regulate diverse cellular physiology at the post-transcriptional level by silencing a natural process of gene silencing. A previous study showed that reversine, a small molecule first developed by Peter G. Schultz group, promotes white adipocyte browning through miR-133a suppression. [34] However, small molecule-based therapies can induce adverse effects because small molecules have an array of targets rather than a specified target, miR-133a in this case. Hence, the miRNA zipper design is optimal for silencing miR-133a to minimize off-target effects. Our hybrid miR-133a zipper NP structure contains an alternating sequence of the fully-hybridized duplex region made of siRNA targeting green fluorescent protein (GFP) and a non-hybridized open region of the miR-133a zipper (bubble region), which are released after cleavage by intracellular dicer or RNases. (FIG. 3A).

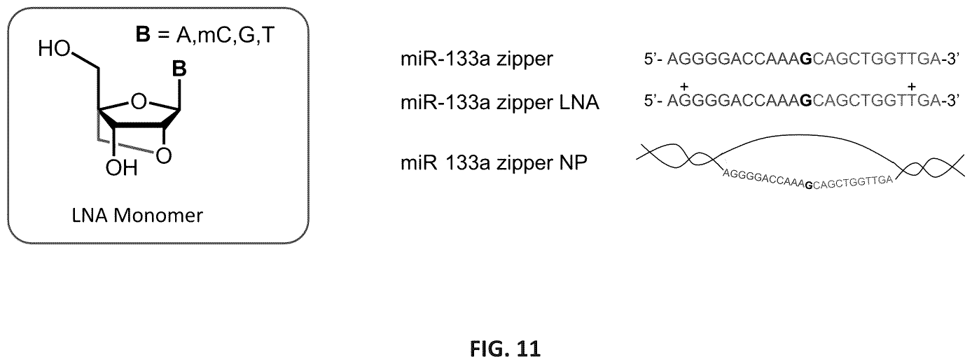

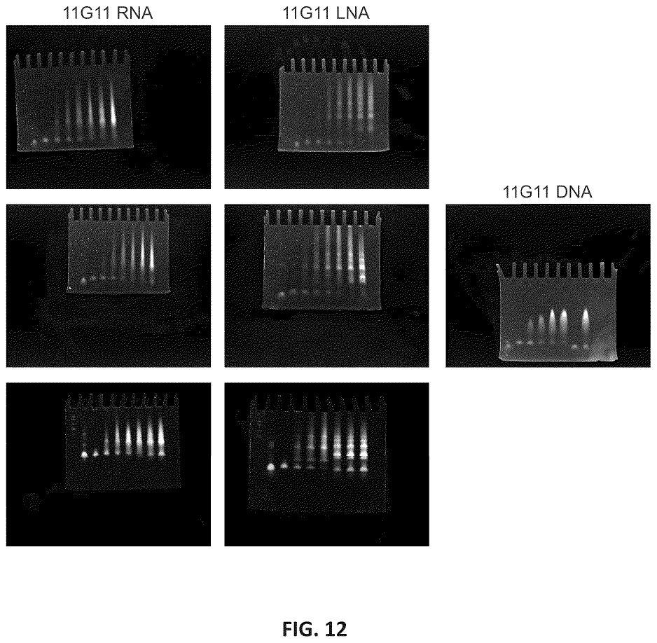

First, we compared target miRNA binding efficiency to evaluate small RNA binding after release from the self-assembled NP and RNA interference. To confirm miRNA zipper formation and compare the binding efficiency of the miR-133a zipper original oligomer, LNA-containing oligomer, zipper NP, and the 5′→3′ miRNA sequence outside of cells, binding to the target sequence was determined using a gel electrophoresis RNA binding shift assay with native polyacrylamide gel electrophoresis (PAGE). LNA is another tool for increasing the stability and affinity of oligonucleotides, such as miR-133a zipper, and exhibits a bicyclic structure in which a 2′-O—CH2-4′ linkage makes a flexible furanose more rigid and resistant to nuclease [8,48] (FIG. 11). Based on evidence that oligonucleotides containing LNA in both ends of the miRNA sequence inhibit mRNA expression efficiently, we designed miR-133a zipper (11G11) to contain LNA at both 3′ and 5′ ends while minimizing self-complementarity (FIG. 11).

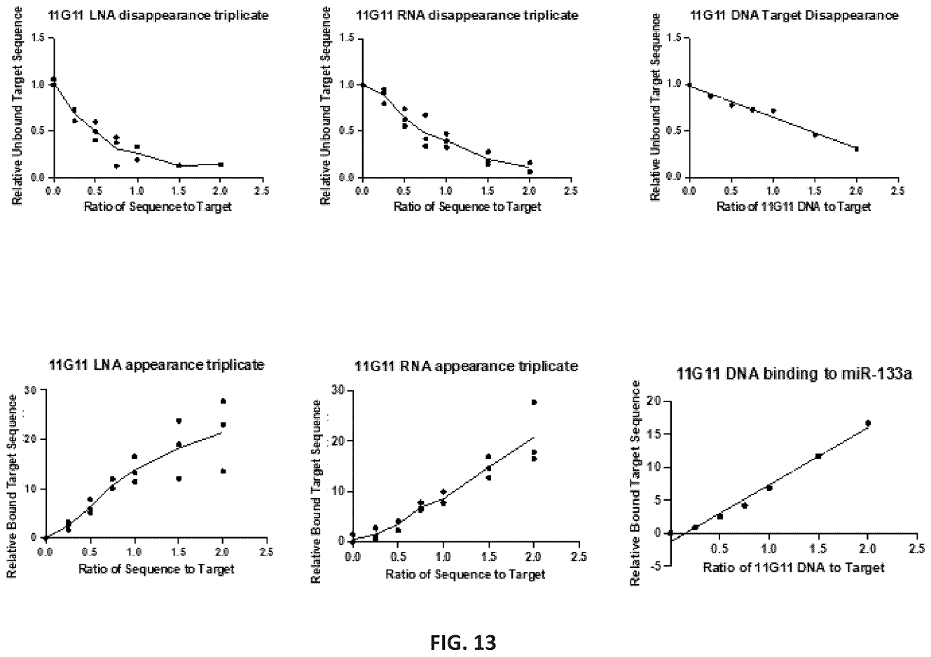

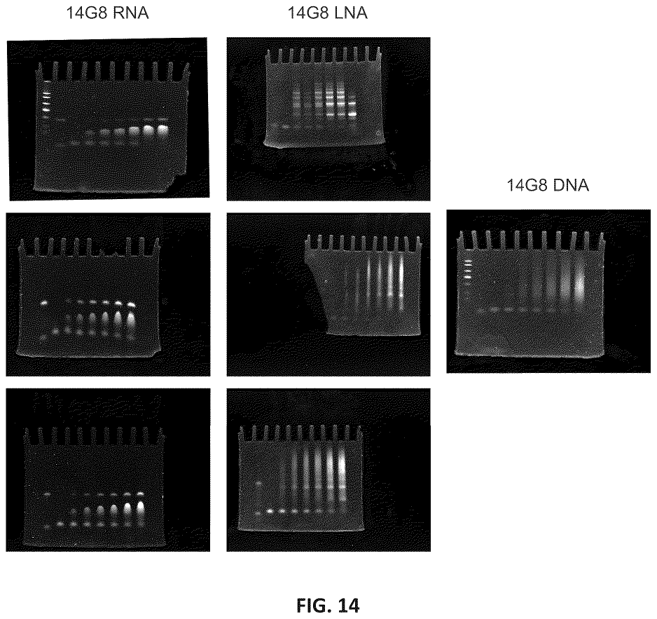

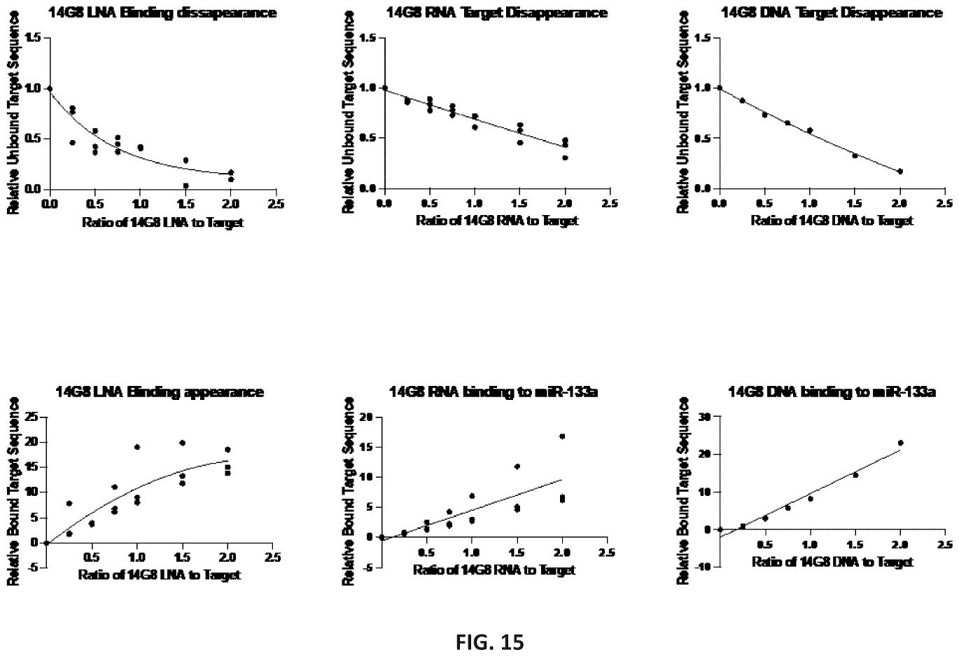

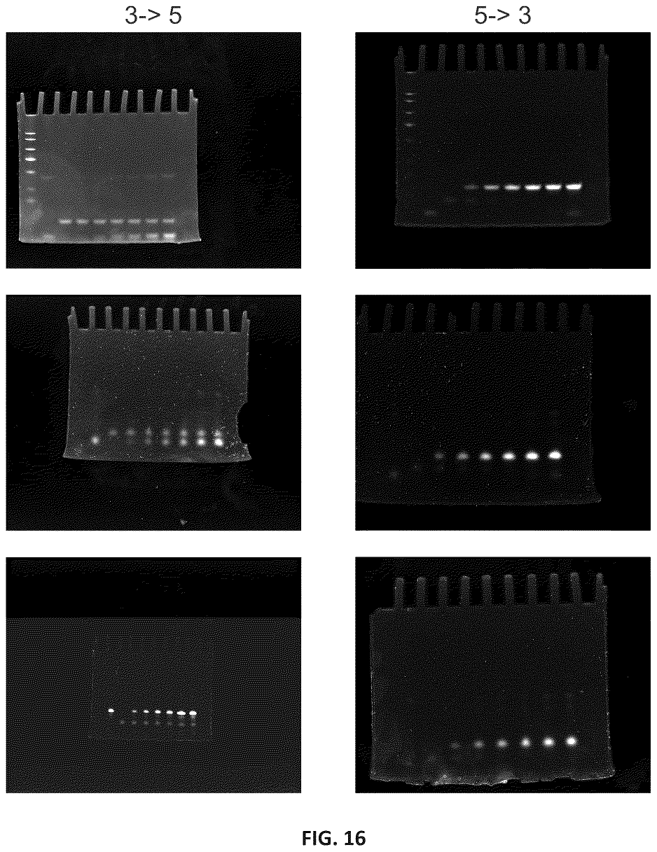

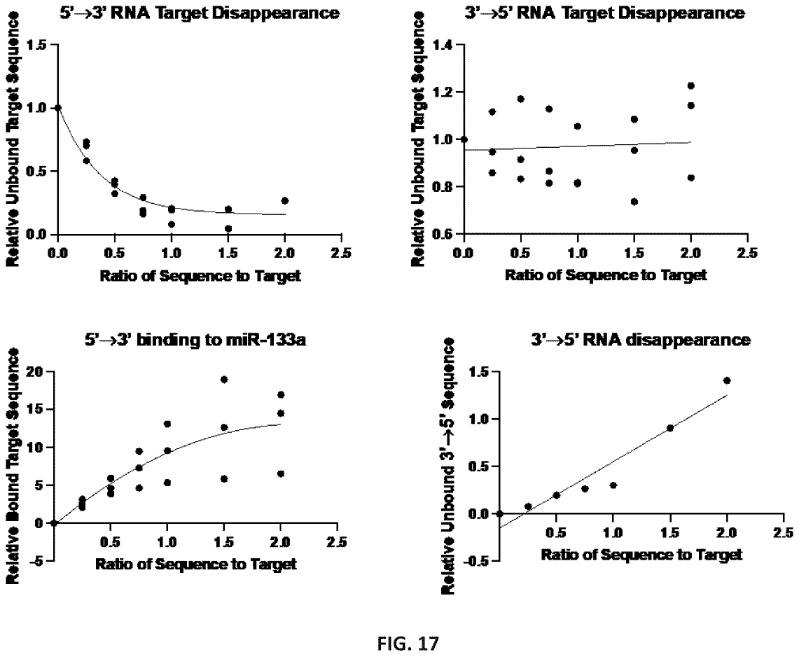

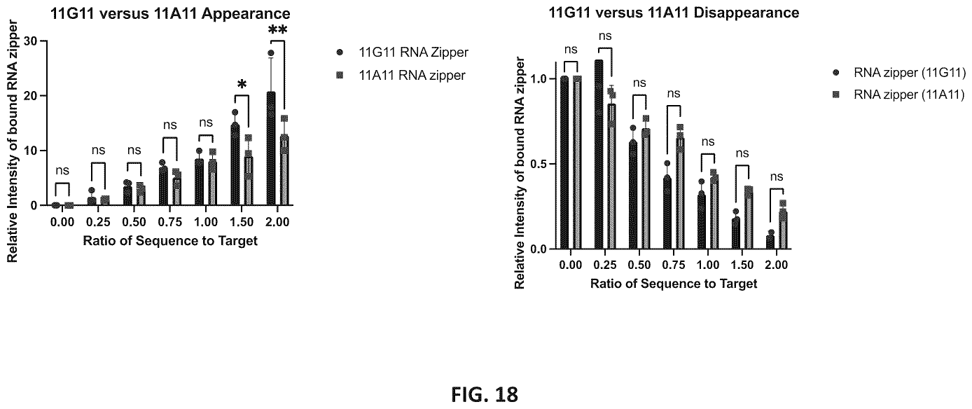

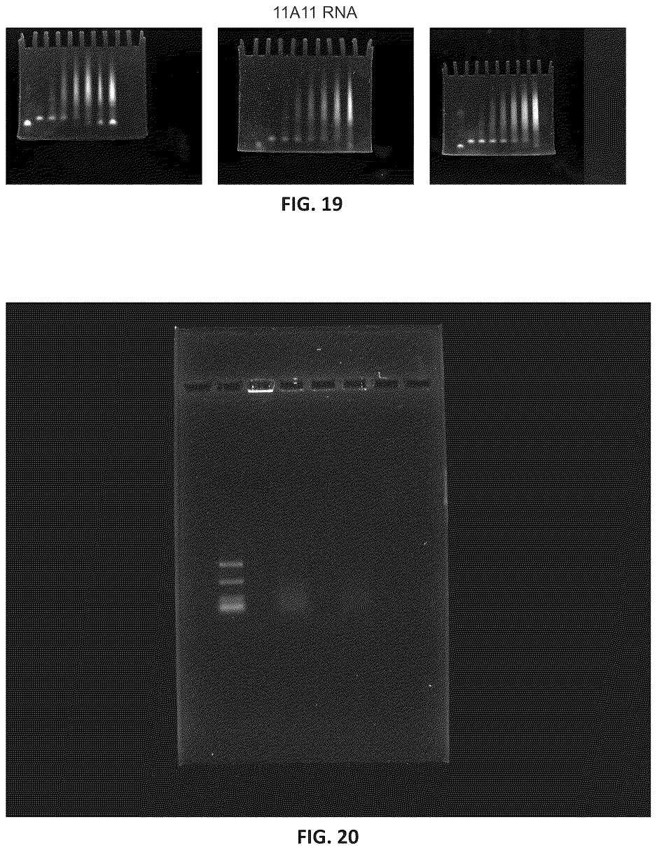

The quantification of both the disappearance of the miR-133a target sequence and the appearance of the bound target was conducted to assess the binding efficiency (FIGS. 3B-C, 12, 14, and 16). Throughout all the lanes, the target weight was held steady, whereas the weight of each binding oligonucleotide was progressively raised, reaching a maximum of twice the target weight. Therefore, the amount of each sequence that binds the miRNA target could be quantified relative to the free sequence and target. The miR-133a zipper sequences, 11G11 and 14G8, had superior binding to the standard 5′→3′ miRNA sequence, based on analysis of the gels for RNA binding kinetics (FIGS. 13, 15, and 17). This can be correlated with the inability of the complete miRNA complement to bind more than a single miR-133a target sequence. Thus, the 5′→3′ miRNA sequence stopped showing an increase in bound product past a 1 to 1 ratio of the target to its complementary sequence. The binding efficiency of the zipper sequence, including LNA, was similar for 11G11 miRNA zipper and slightly higher than the 14G8 miRNA zipper (FIGS. 13, 15, and 17). The efficient binding to the target miRNA sequence outside of cells is critical for the optimal effectiveness of the miR-133a zipper NP. The exponential increase in band intensity at various lengths with the addition of higher RNA, DNA, and LNA-based zipper oligonucleotides to target ratios can be correlated with the formation of different-length zippers. The 3′→5′ miRNA sequence showed no significant binding at any ratio to the target sequence, further illustrating the inefficiencies of standard complementary sequences for RNA binding. The comparable binding of the RNA zipper sequence to the ssDNA sequence gives evidence that the ssDNA template used for RNA transcription is optimal for RNA zipper sequence and NP development. Furthermore, we confirmed an increase in binding efficiency with a G base gap over an A base gap, which we chose for miR-133a zipper NP formation due to sequence structure, as previously discussed (FIGS. 18 and 19).

Targeted Delivery and Intracellular Release of miR-133a Zipper NP in Adipocytes

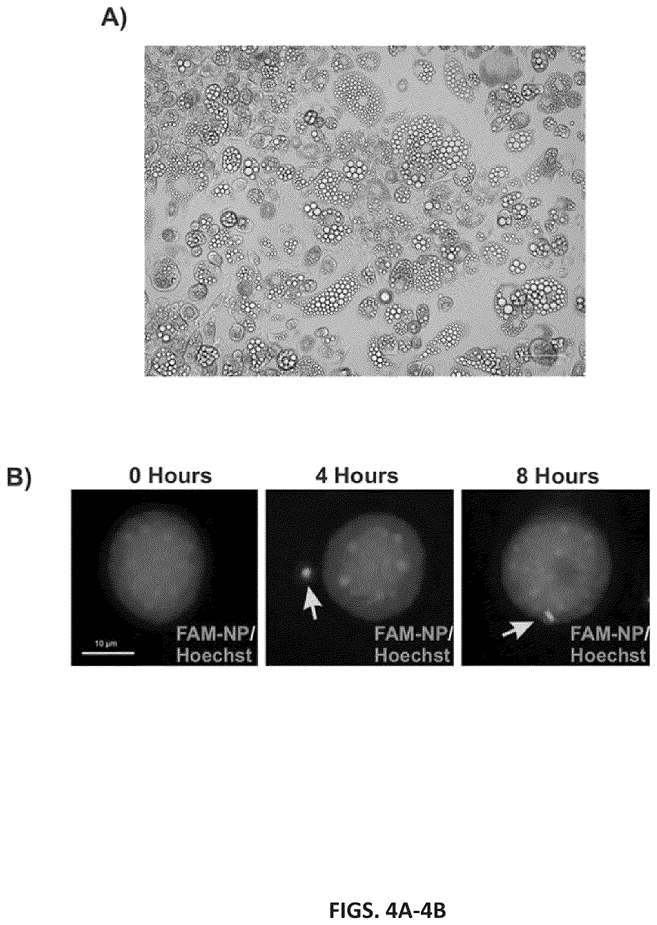

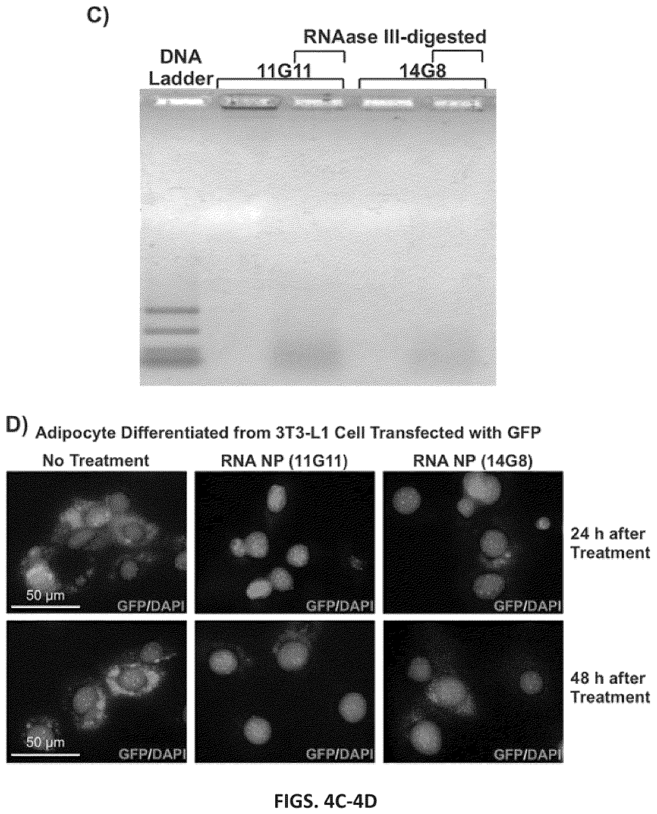

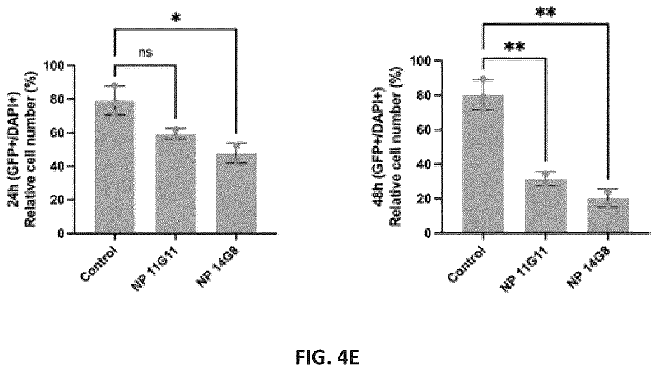

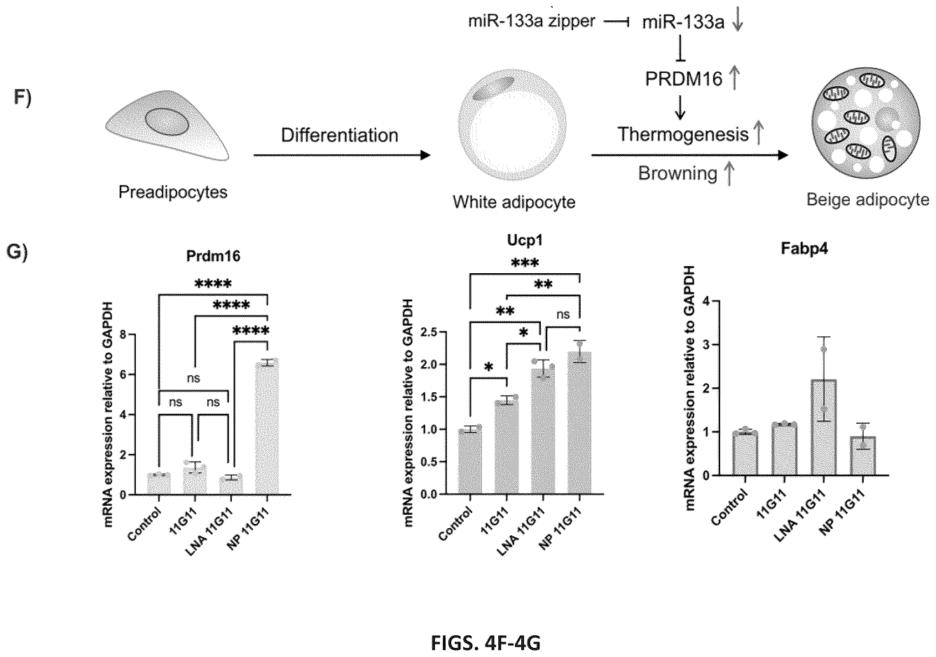

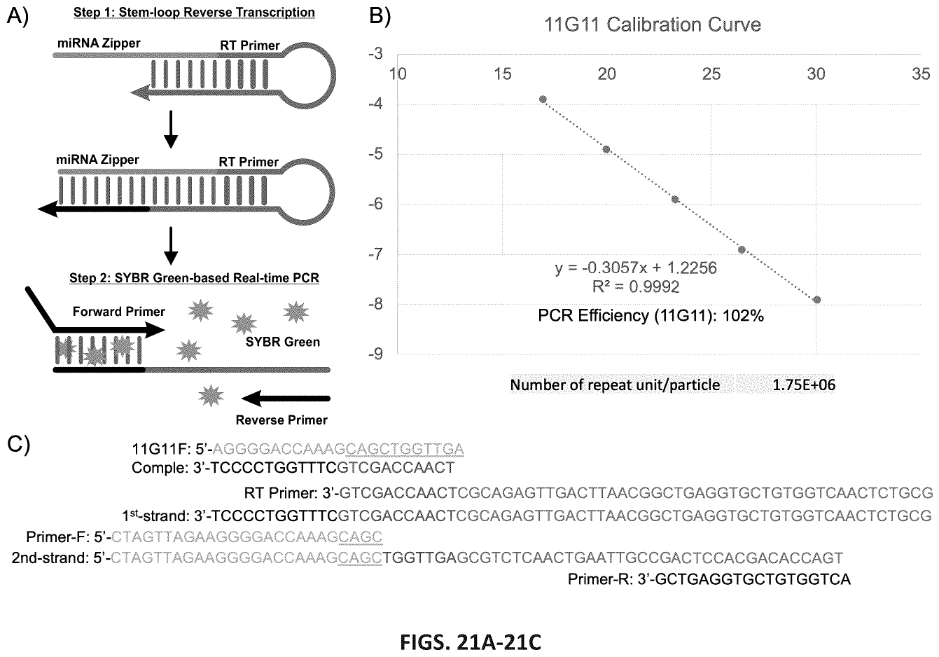

Next, we assessed intracellular delivery of the miR-133a zipper NPs by tracking the fluorescence signal of 6-carboxyfluorescein (FAM)-containing miR-133a zipper NPs in adipocytes differentiated from 3T3-L1 preadipocyte cells (FIG. 4A). The FAM signal was detected near the nucleus of adipocytes 4 h after NP treatment and overlapped with the nuclear membrane after 8 h (FIG. 4B). The data indicates that bubble-structured miR-133a zipper NPs are delivered into the nucleus of adipocytes within 8 h. To verify the release of miRNA zipper and siRNA from the miR-133a zipper NP, native PAGE was run after a dicer cleavage assay, which enabled us to quantify the amount of miRNA zipper released from the NPs over time (FIGS. 4C and 20). We then used RT-qPCR to quantify the amount of miRNA using standard curve quantification. The result shows that the RNA zipper accounted for 8.9% wt. of the miR-133a zip-per NP (FIG. 21). The theoretical amount of zipper in the particle would be 12.9% showing the quantified amount is close to the theoretical amount. Furthermore, based on the 8.9% by weight, 10 nM LNA should deliver the same weight of the miR-133a zipper as 7.36 μg mL⁻¹, 30 nM LNA is equiv. to 20 μg mL⁻¹ miR-133a zipper NP, and 50 nM LNA is equiv. to 36.7 μg mL⁻¹ miR-133a zipper NP. GFP-signal quenching was studied in GFP-expressing adipocytes upon GFP-siRNA release from the miR-133a zipper NP to confirm intracellular cleavage of the small RNA (FIG. 4D). At 24 and 48 h after treatment of adipocytes with the NP, GFP signal intensity was remarkably ablated by siRNA release regardless of miR-133a zipper design (FIG. 4D). The numbers of cells expressing GFP were counted and normalized to the number of DAPI-stained cells. This further confirmed a significant decrease in GFP-expressing cells upon miR-133a zipper NP treatment (FIG. 4E).

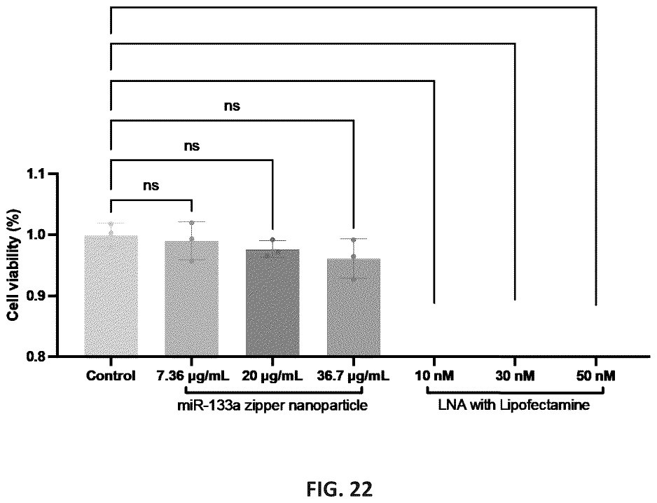

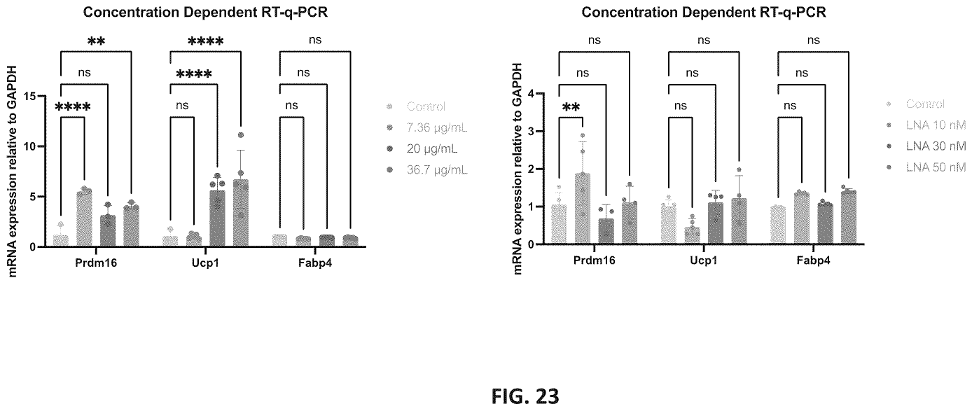

miR-133a directly interacts with Prdm16 mRNA transcript to act as an endogenous suppressor of the expression of Prdm16 gene. [24] Therefore, deleting or antagonizing miR-133a successfully elevated thermogenic gene expression in adipose tissues and browning of mouse adipose tissues. [24,33] In our strategy, after miR-133a zipper NP delivery to adipocytes and cleavage by intracellular RNase, the released miR-133a zippers would freely bind to the miR-133a target, resulting in the induction of Prdm 16-mediated thermogenesis. To assess the stable delivery of the miR-133a zipper with the miR-133a zipper NP and determine therapeutic potential, changes in expression levels for key downstream targets of miR-133a were compared after miR-133a zipper NP delivery or the transfection of miRNA zipper oligonucleotides and LNA-based zipper oligonucleotides, which were designed as illustrated in FIG. 11. The cell viability was tested after delivery of the miR-133a zipper NP or LNA with Lipofectamine, a widely used transfection reagent for in vitro experiments, in a concentration-dependent manner to check cell viability after NP delivery in comparison to control adipocytes and LNA delivered with Lipofectamine (FIG. 22). To evaluate the therapeutic effects of both the LNA zipper and the RNA NP, the same amount of LNA and miR-133a zipper NPs were delivered by weight based on the percentage of miR-133a zipper experimentally determined to be in the NP. To further confirm the therapeutic effects of both the LNA zipper and the RNA NP the same concentrations were tested to confirm RNA expression 24 h after NP delivery. A significant increase in PRDM16 and UCP1 expression was observed with the NP in a concentration dependent manner (FIG. 23). No significant increase was seen in UCP1 with LNA zipper and the PRDM16 upregulation had less significance with LNA zipper than the miR-133a zipper NP. To compare the functional effects of three different compounds, namely the miR-133a zipper original oligomer, LNA-containing oligomer, and NP, expression levels for Prdm 16 and its downstream thermogenic gene, Ucp1 were tested (FIG. 4F). Lipofectamine was used to deliver either the standard zipper sequence or the LNA sequence at a concentration of 50 nM. Notably, the Prdm16 and Ucp1 transcription levels were the highest in adipocytes treated with the miR-133a zipper NP at 20 μg mL⁻¹ (FIG. 4G). However, mature white adipocyte marker (Fabp4) gene expression was not significantly altered by all three developed miR-133a zipper delivery systems, meaning a consistent differentiation rate of white fat-like cells, although the cells displayed browning characteristics with NP delivery (FIG. 4G).

Enhancing oligonucleotide therapeutic intracellular stability has been a critical challenge. Therefore, researchers have studied nucleotide backbone modification as a tool to enhance the stability and affinity of oligonucleotide drugs. LNA is one of the most favorable RNA modifications for RNA therapeutics due to significant improvements in RNA-binding affinity and stability. [48] The first group to suggest the miRNA zipper concept utilized LNA to enhance the zipper oligonucleotide stability and affinity for binding. [22] Nevertheless, our self-assembled NP was more potent than LNA-containing miR-133a zipper. It has been reported that LNA inclusion showed a larger effect on short oligomer (less than 10 bases) binding than longer oligomer binding. [50] Since miRNA and siRNA are usually 21-23 nucleotides in length, [1] our approach to deliver RNAi as a nanostructure is more suitable for RNAi therapeutics than LNA inclusion. Our data suggests that transfection with nascent zipper strands has minimal impact even after prolonged exposure. The RNA design includes double-stranded siRNA segments surrounding the miRNA zipper sequence, creating a more resistant nanostructure to enzymatic degradation. In comparison, single-stranded RNA is more susceptible to enzymatic hydrolysis and is less stable in blood plasma. [20] The improved stability within cells leads to a stronger therapeutic effect, even though the RNA zipper and the LNA zipper sequence have similar efficiencies in binding targets in extracellular environments. NP stability during intracellular delivery may be further linked to the presence of Mg2⁺ ions, which have been shown to stabilize RNA tertiary structure, preventing degradation of the RNA before cellular delivery and cleavage by dicer. [45,46] Therefore, the intracellular delivery rate of miR-133a zipper NP may be higher than nascent oligonucleotides due to its high stability in biological conditions, leading to significant differences in miR-133a zipper effectiveness in cells, although the binding efficiency to the target extracellularly is similar. Thus, the assembly of miR-133a zipper into the NP likely allowed for more significant changes in gene expression than LNA due to the enhanced uptake into cells. Future research that compares the cellular uptake efficiency between LNA and miR-133a zipper NP could determine if the observed increase in gene expression changes in vitro is mainly caused by improved cellular uptake.

### MIR-133a Zipper NP Induces Thermogenic Remodeling of Adipocytes

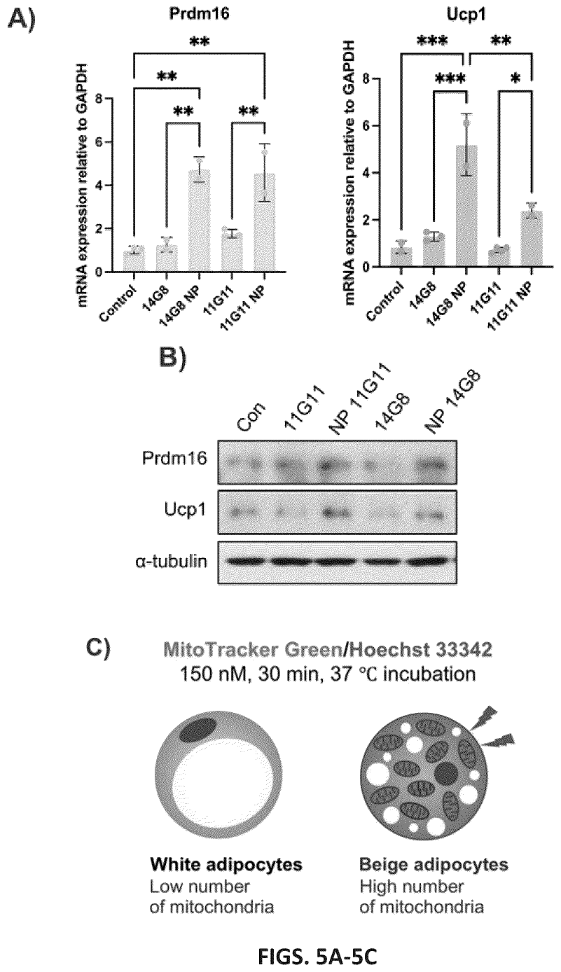

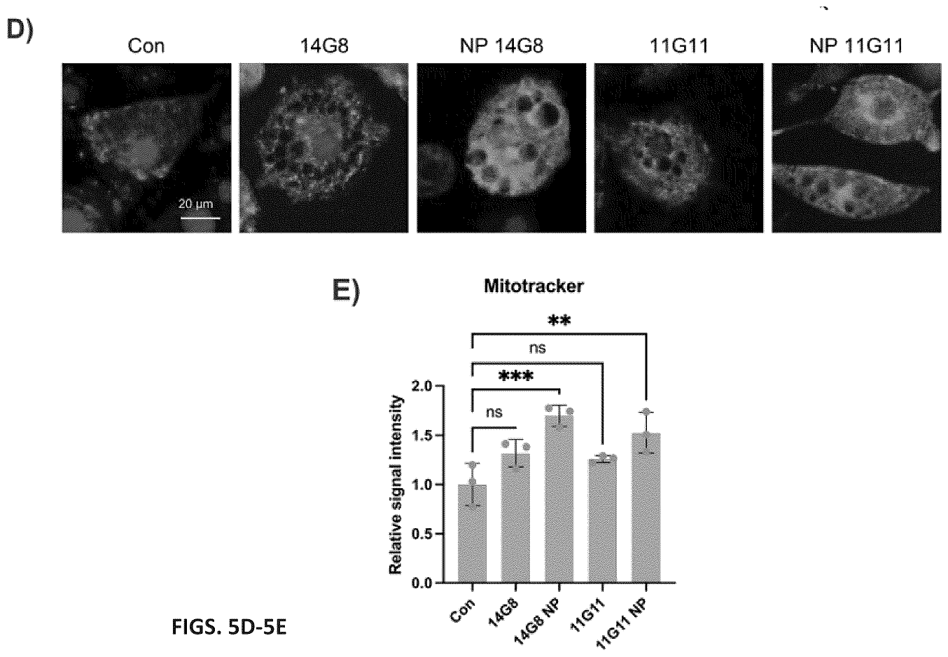

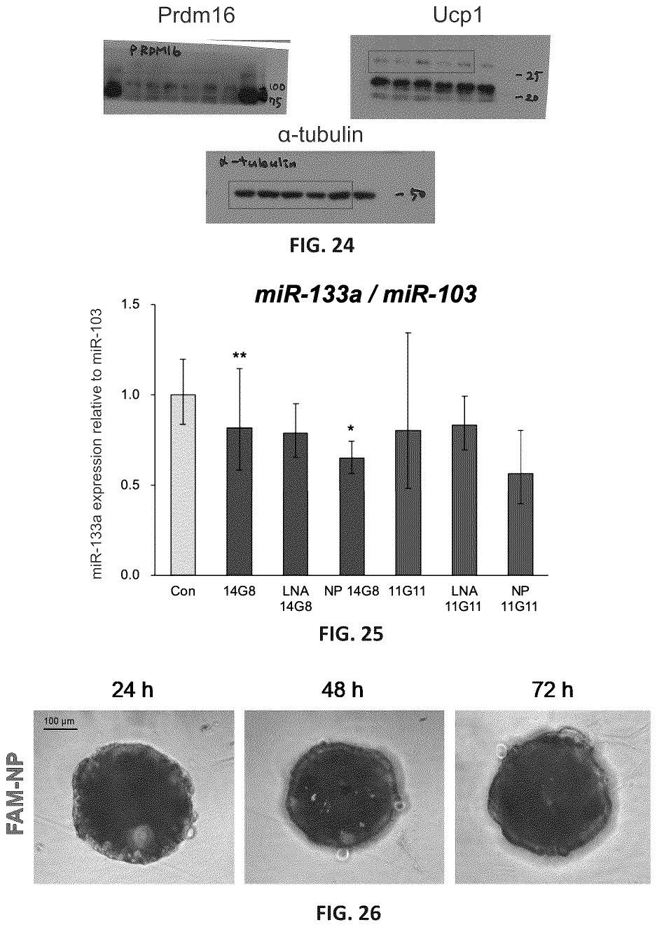

Next, we investigated the influence of the miR-133a zipper sequence on miRNA inhibitory action using 11G11 and 14G8 miR-133a zipper sequences for NP formation. RT-qPCR data showed that both the 11G11 and the 14G8 miR-133a zipper NPs highly enhanced thermogenic gene expression, while transfection with Lipofectamine of original 11G11 and 14G8 miR-133a strands showed minimal effects (FIG. 5A). Interestingly, there was no significant difference in effects between the 11G11 and 14G8 zipper sequences when self-assembled into the NP (FIG. 5A). Similarly, Prdm 16 and Ucp1 protein levels were elevated upon 11G11 and 14G8 NP treatment, as shown with a western blot assay (FIGS. 5B and 24). To assess the targeted binding capability of the miR-133a zipper NP, we conducted qPCR analysis to evaluate the levels of miR-133a in adipocytes (FIG. 25). The knock-down efficiency of miR-133a was ˜50-60% for both NPs, while the transfection of original strands or LNA-containing zipper strands did not significantly reduce miR-133a levels (FIG. 25). The data showed that miR-133a zipper NPs can effectively decrease miR-133a intracellular levels, thus preventing miR-133a-mediated Prdm 16 suppression.

UCP1 mediates non-shivering thermogenesis by converting electrochemical energy into heat in the inner mitochondrial membrane. [51] Thermogenic activation in adipocytes is frequently accompanied by mitochondrial biogenesis, a hallmark of brown fat. [52] Thus, we tested whether the miR-133a zipper NP induces mitochondrial biogenesis in adipocytes. Mitochondrial contents were evaluated with MitoTracker Green staining dye, which stains mitochondria in live cells. A strong green fluorescence signal should be detected in thermogenic beige or brown-like adipocytes containing a high density of mitochondria, while a number of low mitochondria should lead to a weak green signal in non-thermogenic white adipocytes (FIG. 5C). The green fluorescence signal intensity showed a significant increase, as anticipated, when the miR-133a zippers (11G11 or 14G8) were released by the NPs (FIG. 5D). However, the increase in signal intensity was minimal when each original strand was transfected individually (FIG. 5D). Quantification analysis of the mitotracker signal relative to Hoechst 33342 staining the nucleus also showed clear mitochondrial biogenesis by miR-133a zipper NPs (FIG. 5E). The signal intensities in cells transfected with the original zipper strands showed a slight increase, yet remained lower than in cells transfected with the NP, aligning with the unchanged thermogenic gene expression. Collectively, the data presented in FIG. 5 demonstrates that both designs of the miR-133a zipper NP, 11G11 and 14G8, effectively promote the thermogenic activation of adipocytes.

### MiR-133a Zipper NP Reduces the Size of 3D Adipose Spheroids Through Thermogenesis

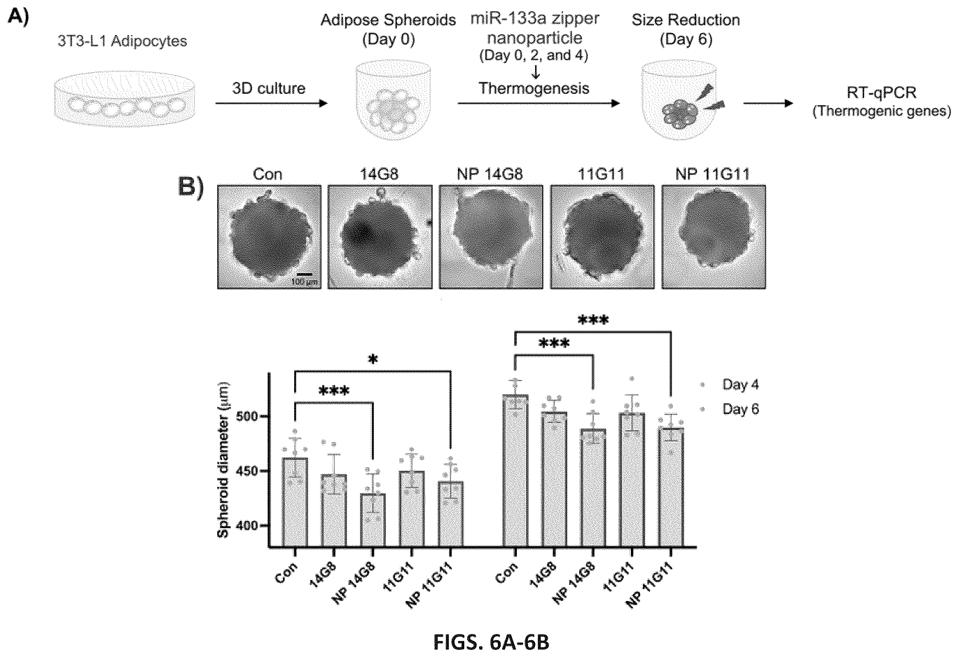

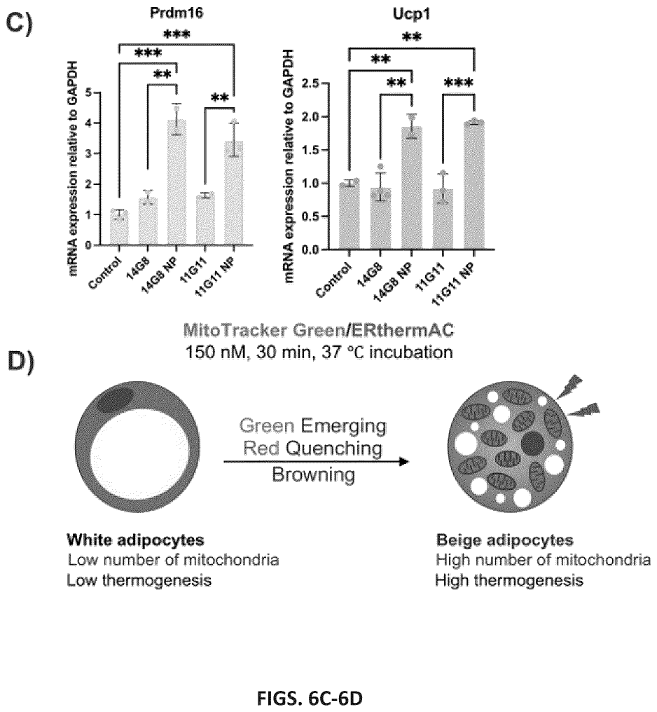

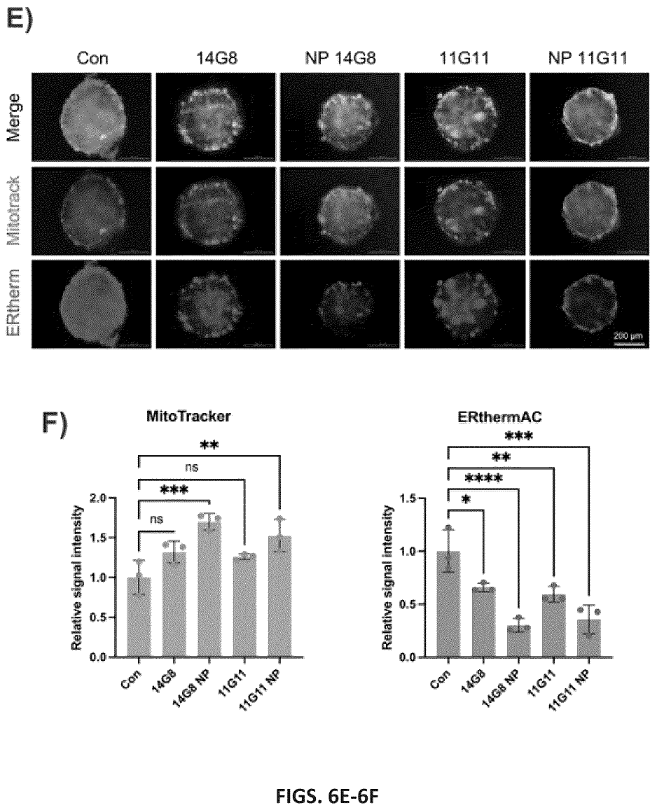

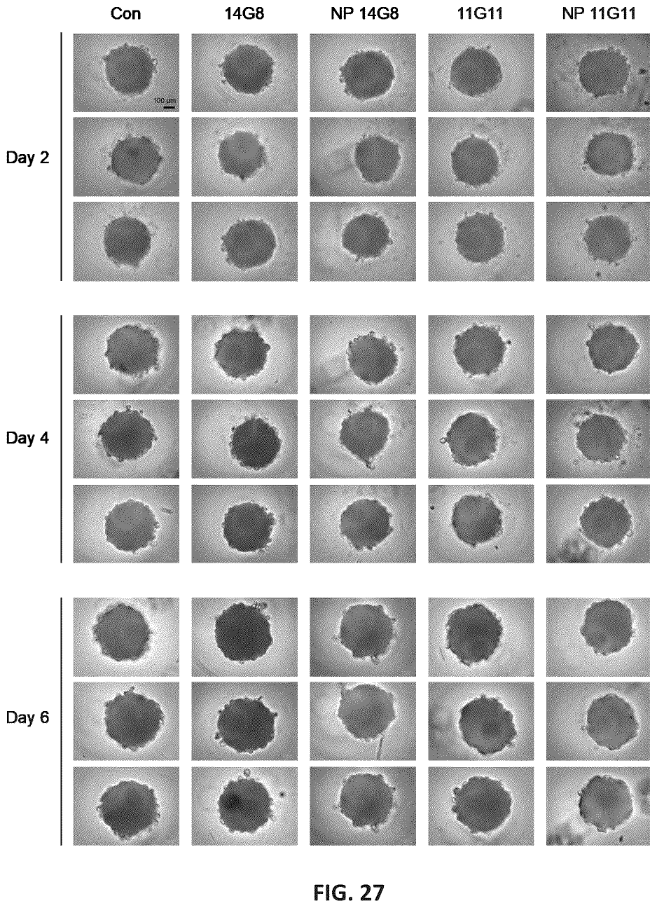

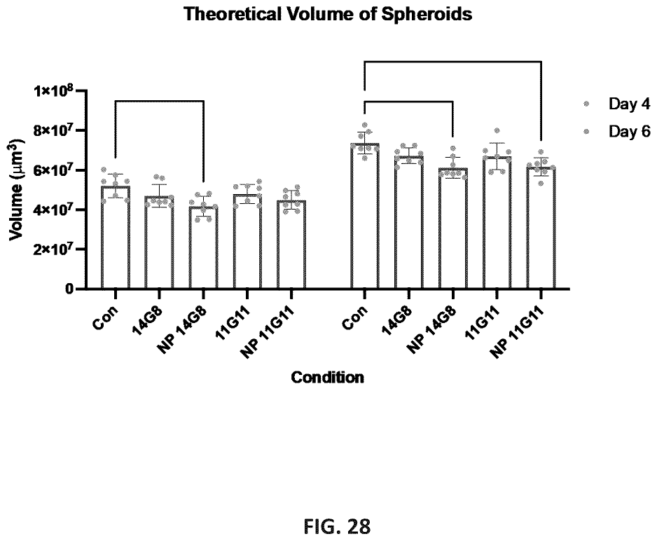

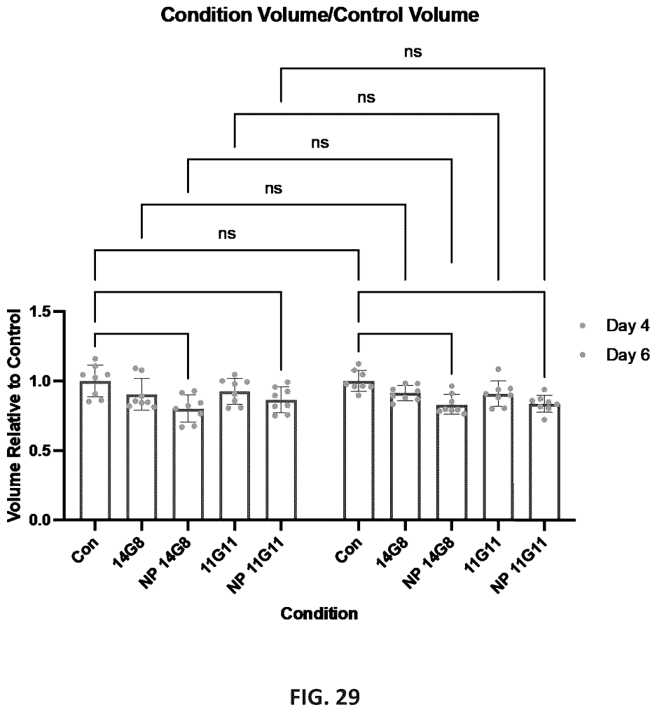

Three-dimensional (3D) adipose spheroids are optimal to recapitulate the morphology and function of in vivo adipose tissues in fat depots. To assess the impact of the miR-133a zipper NP on reducing adipose size, we closely observed the changes in the size of 3D adipose spheroids following the administration of these NPs. Initially grown as a monolayer on the surface of plates, adipocytes were detached and re-aggregated within a 3D culture system. The resulting adipose spheroids were treated with miR-133a zipper NPs or transfected with the original zipper strands on alternate days. 6 days after the initial treatment, the spheroid size was monitored, and RNA was extracted for an RT-qPCR assay (FIG. 6A). Using FAM-tagged miR-133a zipper NPs, we observed NP delivery into the 3D adipose spheroids (FIG. 19). Both miR-133a zipper NPs, 11G11 and 14G8, caused a significant reduction in the diameter of adipose spheroids starting on day 4, while original zipper strands had little effect on adipose spheroid size, even on day 6 (FIGS. 6B, 26, and 27). Furthermore, 14G8 miR-133a zipper NPs led to a more significant decrease in spheroid diameter on day 4 than 11G11 miR-133a zipper NPs, although both NPs had similar significance. The theoretical volume of the spheroids based on the spheroid diameter also shows a decrease from the control (FIG. 28). The ratio of the theoretical volume of the spheroid to that of the control demonstrates consistent effects, maintaining a ratio below 1 without significant changes between days 4 and 6 (FIG. 29). Similarly, Prdm 16 and Ucp1 expression levels were significantly promoted by the zipper system but not by the original strands (FIG. 6C).

Since the expansion of adipose tissue is a key hallmark of obesity, inhibiting adipose tissue growth is a critical parameter in assessing the efficacy of therapeutic interventions to treat obesity. Recent studies illustrate that a 3D culture system of adipocytes is a drug-responsive model that can largely replace animal models. [54,55] Therefore, we assessed the mitochondrial biogenesis and thermogenesis of adipose spheroids for functional validation using two bio-tracker dyes that detect mitochondria and cellular temperature, respectively (FIG. 6D). Upon mitochondrial biogenesis in thermogenically active adipocytes, the green fluorescent signal increased as measured by MitoTracker Green dye fluorescence, while the red fluorescence signal of ERther-mAC dye diminished at high temperatures in thermogenic adipocytes (FIG. 6E). After exposure to NPs or the original miR-133a zipper strands for 6 days, the adipose spheroids were simultaneously stained with MitoTracker Green and ERthermAC. As expected, green signals indicative of the mitochondria were significantly increased, and red signals were quenched upon 14G8 or 11G11 miR-133a zipper NP treatment (FIG. 6F). These signal alterations were also observed in spheroids transfected with the original zipper oligonucleotides, although it was not statistically significant for MitoTracker and led to a less significant increase in thermogenesis than either NP (FIG. 6F). The experimental data with 3D adipose spheroids described above indicates that our miR-133a zipper NP can promote thermogenesis and reduce adipose size in tissue-like 3D structural conditions of the cells, confirming the browning effects of miR-133a zipper NP.

The comparability in miR-133a zipper NP effectiveness across different cell models points toward the potential of the NP's ability to create consistent changes to gene expression, an essential factor in RNA therapeutics. Furthermore, considering that miR-133a zipper NPs effectively reduced adipose spheroid size, it is expected that our NPs could reduce adipose mass in clinical use to treat obesity. When we use this ex vivo 3D culture system, we cannot exclude the possibility that only the cells located on the surface of spheroids are affected by the miR-133a zipper. However, vasculature formation occurs throughout adipose tissues in physiological conditions, so miR-133a zipper NP should theoretically be delivered into the inner mass of adipose tissues due to vasculature in vivo conditions. Furthermore, scavenger receptor B1, previously shown to enhance uptake in spheroids, may also play a significant role in the uptake of NPs into adipocytes, as suggested by prior research.

### CONCLUSION

While many oligonucleotide delivery platforms for targeted gene therapy have been developed, [49] achieving RNAi delivery with high affinity, specificity, and stability remains a complex challenge. In this study, we have introduced an innovative miRNA-modulating tool that enhances affinity, specificity, and stability by integrating miRNA zipper technology and RCT-based RNA NP technology. This tool employs a small oligonucleotide zipper that binds two miR-133a molecules via complementary interaction with their 5′ and 3′ ends. The zipper's nucleotides capture endogenous miRNA through complete complementary base pairing, although its sequence differs from miR-133a, with reversed 5′ and 3′ regions at either 11 nucleotides (11G11) or 14 nucleotides (14G8) from the 5′ end. Leveraging these features, we could inhibit miR-133a action with high affinity and specificity by incorporating it into a template along with GFP siRNA, creating a bubble-like structure that self-assembles into an miR-133a zipper NP. This facilitates the delivery of the miRNA zipper into both monolayered adipocytes and adipose spheroids. This work showcases the potential of self-assembled miR-133a zipper NPs in delivering miR-133a zipper to adipocytes for therapeutic effects and for siRNA and the miRNA zipper co-delivery. Upon delivery into adipocytes, the miR-133a zipper is released, effectively binding endogenous miR-133a and promoting the expression of its target, Prdm16. We observed that treatment with the miR-133a zipper NP enhanced thermogenic gene expression and mitochondrial biogenesis in both monolayered adipocytes and adipose spheroids. In adipose spheroids, which more closely mimic in vivo conditions, we observed a reduction in adipose mass and an increase in cellular temperature following treatment with the miR-133a zipper NP. As the miR-133a zipper NP comprises only naturally biodegradable materials, our approach ensures biocompatibility and significantly reduces potential immune responses or other adverse effects associated with foreign materials such as LNPs, viruses, or inorganic substances.

Another important issue in gene therapy is tissue-specific targeting to prevent unintended physiological outcomes in non-pathogenic regions. miR-133a-mediated suppression of PRDM16 was selected as the target to introduce cell type specificity into the molecular mechanism of interaction with PRDM16. Studies show that direct PRDM16 overexpression can induce severe adverse effects derived from uncontrolled thermogenesis in other tissues and white fat depots. Experimentally, the injection of dinitrophenol (DNP), a classical mitochondrial uncoupling agent, causes heat production in all cells of the body, leading to uncontrolled hyperthermia. [57,58] Unlike this conventional method using artificial uncouplers, our approach represses the action of an endogenous Prdm16 inhibitor, miR-133a, thereby inducing thermogenesis only in adipose tissues or muscle where miR-133a mainly functions. MiR-133a inhibition has been shown to selectively induce PRDM16 overexpression in muscle and fat cells, making it an optimal selection for PRDM16 targeting. [57] With this approach, the thermogenic activity of the miR-133a inhibiting NP is limited to the innate action of miR-133a-mediated Prdm16 suppression, which occurs in fat and muscle cells, thus preventing excessive heat production in other tissue types and leading to off-target effects. Although miR-133a is mainly expressed in adipocytes, miR-133a zipper NPs could induce side effects when delivered to skeletal muscle or cardiac muscle, which could be further studied through in vivo testing. One previous literature generated miR-133a1^{-/-}a2^{+/-} mice that have three out of the four miR-133a alleles knocked out, and those mice had normal cardiac and skeletal muscles, while the knock out resulted in browning of adipose tissues. These findings introduce a novel RNAi platform that leverages a specially designed miR-133a zipper NP. In short, our developed platform holds great promise for anti-obesity treatments through the generation of thermogenic adipocytes. This technology has great potential for broader applications in various diseases, combining miRNA zipper technology with siRNA delivery for multifaceted therapeutic approaches. Overall, our innovative platform, leveraging meticulously designed self-assembled RNAi nanotechnology, not only holds great promise for anti-obesity treatments, but also has great potential for broader applications in various diseases that combine miRNA zipper technology with siRNA delivery for multifaceted therapeutic approaches.

### Experimental Section

Preparation of cDNA Templates: A 5′-phophorylated linear DNA (PAGE purified, 85-base pair, 10 μM) was heated with a primer DNA containing T7 promoter sequence (22-base pair, 10 μM) in Duplex Buffer (30 mM HEPES, pH 7.5, 100 mM potassium acetate) (Integrated DNA Technologies, USA) at 95° C. for 2 min, then let cool down slowly to room temperature. After 1 h, the reaction vessel was added to the final concentration of 1×T4 buffer (50 mM Tris-HCl, 10 mM MgCl₂, 1 mM ATP, 10 mM DTT, pH 7.5) (New England Biolabs, USA), 2.5 mM MnCl₂ (Sigma-Aldrich, USA), and 200 U μL⁻¹ T4 DNA ligase (New England Biolabs, USA) to initiate the ligation of the nick DNA and form circularized DNA template. The ligation reaction was carried out at 16° C. for 18 h), and then the T4 DNA ligase was inactivated at 65° C. for 10 min. To remove linear free DNA from the reaction, the ligation reaction was diluted 1:10 and incubated with exonuclease 1 and 3 (New England Biolabs, USA) at 37° C. for 30 min. Subsequently, the reaction was stopped by adding EDTA to 11 mM, followed by heat inactivation at 80° C. for 30 min. The cDNA templates were used for subsequent steps without further purification.

TABLE 1
Linear DNA and primer DNA sequences

| Oligonucleotides | Sequences (5′-3′) |
|---|---|
| T7 Primer | TAATACGACTCACTATAGGGAT (SEQ ID NO: 1) |
| Template for | /5Phos/ATAGTGAGTCGTATTAAAGCAAGCTGACCCTGAAGTCAA |
| miR-133a-Zipper | CCAGCTGCTTTGGTCCCCTGTGGAGCCTATTGCTTCGATTATCCC |
| 11G11 | T (SEQ ID NO: 2) |
| Template for | /5Phos/ATAGTGAGTCGTATTAAATCGAAGCAATAGGCTCCACGTG |
| complementary | TCATGAGACGGTCGAACCACCTTCAGGGTCAGCTTGCTTATCCC |
| sequence of | T (SEQ ID NO: 3) |
| 11G11 | |
| Template for | /5Phos/ATAGTGAGTCGTATTAAAGCAAGCTGACCCTGAAGACCA |
| miR-133a-Zipper | GCTGCTTTGGTCCCCTTCAGTGGAGCCTATTGCTTCGATTATCCC |
| 14G8 | T (SEQ ID NO: 4) |
| Template for | /5Phos/ATAGTGAGTCGTATTAAATCGAAGCAATAGGCTCCACCAC |
| complementary | GTGTCATGAGACGGTCGAGTCTTCAGGGTCAGCTTGCTTATCCC |
| sequence of 14G8 | T (SEQ ID NO: 5) |
| FAM labeled Tag | /56-FAM/TCCCTATAGTGAGTCGTATT/3AmMO/ (SEQ ID NO: 6) |
| Ucp1 primer | Forward: ACTGCCACACCTCCAGTCATT (SEQ ID NO: 7) |
| sequences | Reverse: CTTTGCCTCACTCAGGATTGG (SEQ ID NO: 8) |
| Prdm 16 primer | Forward: CAGCACGGTGAAGCCATTC (SEQ ID NO: 9) |
| sequences | Reverse: GCGTGCATCCGCTTGTG (SEQ ID NO: 10) |
| Fabp4 primer | Forward: AAGGTGAAGAGCATCATAACCCT (SEQ ID NO: 11) |
| sequences | Reverse: TCACGCCTTTCATAACACATTCC (SEQ ID NO: 12) |
| Gapdh primer | Forward: TGGCCTTCCGTGTTCCTAC (SEQ ID NO: 13) |
| sequences | Reverse: GAGTTGCTGAAGTCG (SEQ ID NO: 14) |
| 11A11-zipper | AGGGGACCAAAACAGCTGGTTGA (SEQ ID NO: 15) |
| sequence | |
| 11G11-zipper | AGGGGACCAAAGCAGCTGGTTGA (SEQ ID NO: 16) |
| sequence | |
| 14G8-zipper | TGAAGGGGACCAAAGCAGCTGGT (SEQ ID NO: 17) |
| sequence | |
| miR-133a target | TTTGGTCCCCTTCAACCAGCTC (SEQ ID NO: 18) |
| sequence | |
| sequence | AAACCAGGGGAAGTTGGTCGAC (SEQ ID NO: 19) |
| complementary to | |
| miR-133a target | |
| RT primer for | GTCGACCAACTCGCAGAGTTGACTTAACGGCTGAGGTGCTGTG |
| miR-133a | GTCAACTCTGCG (SEQ ID NO: 20) |
| quantification | |
| Primer-F and | Primer-F: CTAGTTAGAAGGGGACCAAAGCAGC (SEQ ID NO: 21) |
| primer-R for | Primer-R: GCTGAGGTGCTGTGGTCA (SEQ ID NO: 22) |
| miR-133a | |
| quantification | |

Synthesis of MiR-Zipper NPs: The miR-Zipper NPs were prepared by RCT using HiScribe T7 High Yield RNA Synthesis Kit (New England Biolabs, USA). To a 200 μL reaction, an equimolar amount of the two circularized DNA templates containing complementary sequences was added to the solution mixture containing T7 Reaction Buffer, ribonucleotides, and T7 RNA polymerase. The RCT reaction was conducted at 37° C. for 20 h. Then, the miR-Zipper NPs were purified by centrifugation and washed with nuclease-free water (QIAGEN, USA) 3 times and stored at 4° C. until further use. To fluorescently label miR-zipper NPs, 6-carboxyfluorescein (FAM)-labeled oligonucleotides (Integrated DNA Technologies, USA) containing complementary sequences to a non-functional portion of miR-zipper NPs were annealed to the particles at a final concentration of 10 μM at 65° C. for 10 min.

Analysis of Circularized DNA Templates by Agarose Gel Electrophoresis: The formation of circularized DNA templates was confirmed using agarose gel electrophoresis. Briefly, agarose gel was prepared by heating 1% agarose solution in Tris-acetate-EDTA (TAE) buffer, and then GelRed Nucleic Acid Gel Stain (Biotium) was added before gel solidified. The samples were mixed with 6×orange loading buffer (ThermoFisher Scientific, USA) prior to loading into the gel and electrophoresed at 90 V for 40-60 min. The gel images were visualized using a UV transilluminator (ThermoFisher Scientific, USA).

Characterization for NPs: For morphological characterization, a field emission scanning electron microscope (FE-SEM, Zeiss, Germany), a transmission electron microscopy (TEM, JEOL 1200EX electron microscope with AMT-XR41 digital camera, Japan), and an atomic force microscope (AFM, Park systems, NX10) were used to obtain morphology of the miR-Zipper NPs, while energy dispersive x-ray spectroscopy (FE-SEM with the EDS detector) was used for elemental composition. NP tracking analysis (NTA, NanoSight NS300, Malvern Panalytical Inc.) was used for size and number concentration characterization. The miR-Zipper NPs were sampled for FE-SEM and AFM by deposited on 10 mm VI mica discs with 0.21 mm thickness (Ted Pella, Inc., USA, Cat. No. 50). For FE-SEM analysis, the air-dried samples were coated with gold (Au) before imaging. For AFM imaging, 10 μL of the reaction mixture was diluted in nuclease-free water containing 5 mM Tris-HCl and 5 mM MgCl₂. After incubating the mixture at 4° C. for 30 min, 50 μL of the mixture was deposited onto the freshly cleaned mica surface, and further incubated at 4° C. for 30 min. Following the incubation, the mica surface was rinsed with deionized water to remove salts, and nitrogen gas was then sprayed onto the surface for three to five seconds to remove the remaining solution. The samples were scanned in non-contact mode with NC-NCH tips (Park Systems). For FE-SEM with EDS, 20 μL of the purified miRNA-133a zipper NPs were drop casted onto carbon tape and dried overnight in the desiccator. They were coated with platinum for SEM imaging and EDS analysis. For TEM analysis, the samples were deposited on carbon-coated copper (Cu) grids (Ted Pella Inc., USA), and then dried under vacuum before analysis. Hydrodynamic size of the NPs was measured on Malvern Instruments Zetasizer Nano ZS-90 (Malvern, USA).

SIRNA and MiR-Zipper Release from NP: To quantify the amount of siRNA and miR-Zipper molecules, a known number of miRNA-Zipper NPs measured using Nanodrop (ThermoFisher Scientific, USA) were incubated with recombinant dicer (Genlantis, USA) in a 10 μL reaction solution containing 1 μg of miR-Zipper NP, 1 mM ATP, 2.5 mM MgCl2, 40% Dicer Reaction Buffer, 1U Recombinant Dicer Enzyme. The solution was incubated for different time periods (12 to 48 h) at 37° C. before adding Dicer Stop Solution to deactivate the Dicer enzyme. Generated siRNA molecules were then confirmed by gel electrophoresis with 3% agarose gel.

Quantification of SiRNA and MiR-Zipper Release: The generated miRNA-Zipper molecules were quantified using a stem-loop RT-qPCR method adopted from the literature to quantify small RNA. [59,60] The 11G11 and 14G8 miRNA-Zipper RNA molecules (Integrated DNA Technologies, USA) were used as standards for absolute quantification using SYBR Green Master Mix (ThermoFisher Scientific, USA) on a StepOnePlus real-time PCR System (Applied Biosystems, USA). All measurements were done in triplicate.

Native PAGE for Studying RNA Release from NP and RNA Binding: The same amount of target was incubated in nanopure water with each of sequences in increasing concentration at 60° C. for 20 min, followed by 20 min incubation at room temperature. Samples were prepared with gel loading buffer. To compare the binding efficiency of the NP and the target to other sequences, a 10% native PAGE was run at 10 V cm 2 for 2.5 h at 4° C. The gel was stained and imaged. Quantification of band intensity was performed with the Thermo Fisher iBright imaging analysis software. Cell Culture and Differentiation: 3T3-LI preadipocyte cell line was purchased from ATCC (Cat. No. CL-173). The cells were grown in high-glucose Dulbecco's modified Eagle medium (DMEM, Welgene, Cat. No. LM001-05) supplemented with 10% bovine calf serum (BCS, Welgene, Cat. No. S103-01) and 1% penicillin/streptomycin (P/S, Welgene, Cat. No. LS202-02) at 37° C. in humidified 5% CO2 incubator. For differentiation into mature adipocytes, 80% confluent 3T3-LI cells were incubated in high-glucose DMEM with 10% fetal bovine serum (FBS, Welgene, Cat. No. S001-07), 1% P/S, 0.5 mM 3-isobutyl-1-methylxanthine (IBMX, Sigma, Cat. No. 15879), 1 μM dexamethasone (Sigma, Cat. No. D1756), and 10 μg mL⁻¹ insulin (Sigma, Cat. No. 19278) for 2 days. Then, the cells were incubated in high-glucose DMEM with 10% FBS, 1% P/S, and 10 μg mL⁻¹ insulin for further 6-8 days. The medium was changed every other day.

Adipose Spheroids Generation: Fully differentiated adipocytes in monolayer were detached from plates using Trypsin-EDTA solution. After dissociation, the cells were reaggregated in 96 well round bottom low adhesion plates (10 000 cells per well) in DMEM supplemented with 10% FBS and 1% P/S. 2 days after, adipose spheroids were transfected with miR-133a zipper or treated with RNA NP.

Transfection: Monolayered adipocytes or adipose spheroids were transfected with the original miR-133a zipper oligos (final 50 nM) using Lipofectamine 3000 reagent (Invitrogen, Cat. No. L3000001) according to the manufacturer's instruction. For monolayered adipocytes, transfection was performed twice for 4 days (once every two days). For adipose spheroids, transfection was performed three times for 6 days (once every two days). The sequences of miR-133a zipper oligos are as follow: 14G8, 5′-TGAAGGGGACCAAAGCAGCTGGT-3′ (SEQ ID NO: 17), 11G11,5′-AGGGGACCAAAGCAGCTGGTTGA-3′ (SEQ ID NO: 16), LNA 11G11, 5′-A+GGGGACCAAAGCAGCTGGT+TGA-3′ (SEQ ID NO: 16).

Analysis of RNA NP-Mediated GFP Knockdown: To evaluate the effects of siRNA targeting eGFP released from RNA NPs, differentiated adipocytes were transfected with pcDNA3-EGFP (Addgene, Cat. No. 13 031) using Lipofectamine 3000 reagent. After 6 h, the adipocytes were treated with miR-133a zipper NP (final concentration 20 μg mL 1). 24 and 48 h after the treatment, the GFP signal was detected using Cytation 5 Cell Imaging Multi-Mode Reader (BioTek).

Reverse Transcription (RT)-Quantitative Polymerase Chain Reaction (qPCR): Total RNA was extracted from monolayered adipocytes or adipose spheroids using the Easy-Blue reagent (Intron, Cat. No. 17061). 1 μg of RNA was reverse transcribed into cDNA using the Maxime RT PreMix kit (Intron, Cat. No. 25081). qPCR was performed using KAPA SYBR FAST qPCR (Roche, Cat. No. KK4601) with a CFX96TM real-time PCR detector (Bio-Rad). Relative mRNA levels were normalized to B-actin or Gapdh mRNA levels for each target gene. The PCR primer sequences used are as follow: Gapdh forward, 5′-ATGACATCAAGAAGGTGGTG-3′ (SEQ ID NO: 23), Gapdh re-verse, 5′-CATACCAGGAAATGAGCTTG-3′ (SEQ ID NO: 24), Prdm 16 forward, 5′-CAGCACGGTGAAGCCATTC-3′ (SEQ ID NO: 9), Prdm16 reverse, 5′-GCGTGCATCCGCTTGTG-3′ (SEQ ID NO: 10), Ucp1 forward, 5′-ACTGCCACACCTCCAGTCATT-3′ (SEQ ID NO: 7), Ucp1 reverse, 5′-CTTTGCCTCACTCAGGATTGG-3′ (SEQ ID NO: 8), Fabp4 forward, 5′-AAGGTGAAGAGCATCATAACCCT-3′ (SEQ ID NO: 11), Fabp4 reverse, 5′-TCACGCCTTTCATAACACATTCC-3′ (SEQ ID NO: 12).

Quantification of MiRNA via RT-qPCR Analysis: The intracellular miRNA-133a level was quantified in each treatment condition from the total RNA extracted from culture using TRIZol reagent (Invitrogen, USA). The total RNA (100 ng) was subsequent to reverse transcription to form cDNA using miRCURY LNA RT kit (QIAGEN, USA). The cDNA was used as a template for real-time PCR analysis using miR-133a-specific assays in a miRCURY LNA miRNA PCR system (QIAGEN, USA), following the manufacturer's protocol, on a StepOnePlus real-time PCR System (Applied Biosystems, USA). The mmu-miR-133a-3p were used as miR-133a-specific primers and mmu-miR-103a-3p as the endogenous control. The two-step cycling conditions were performed as follows: Initiation activation at 95° C. for 2 min, 40 cycles of denaturation at 95° C. for 10 s, and combined annealing/extension at 56° C. for 60 s. The resulting CT values were normalized and reported in fold changes relative to the endogenous control (miR-103a-3p). All measurements were repeated three times.

Western Blot Assay: Protein lysates of adipocytes were extracted with Pro-prep reagent (Intron, Cat. No. 17081) for 1 min on ice and then centrifuged at 13,000 rpm at 4° C. for 20 min. The protein concentration of supernatants was measured using Bradford assay. 15 μg of proteins were subjected to sodium dodecyl sulfate-polyacrylamide gel electrophoresis (SDS-PAGE) (FIG. 24). Subsequently, the separated proteins were transferred onto polyvinylidene difluoride membranes (Millipore, Cat. No. IEVH00005) using a semi-dry transfer system (Bio-Rad). Then, the membranes were incubated with primary antibodies (Prdm16, Abcam, Cat. No. ab106410, Ucp1, Abcam, Cat. No. ab 10983, α-tubulin, Santa Cruz, Cat. No. SC-32293) at 4° C. for 16 h on the shaker, followed by incubation with horseradish peroxidase (HRP)-conjugated secondary antibodies at room temperature for 1 h. The HRP signals were detected with chemiluminescence reagent (Absignal, Cat. No. ABC-3001) on AGFA 100 NIF X-ray film.

Mitochondrial Staining: Monolayered adipocytes or adipose spheroids were incubated with 200 nM of MitoTracker Green FM (Cell Signaling Technology, Cat. No. 9074) for 30 min at 37° C. For staining the nucleus of adipocytes, Hoechst 33342 solution was also added with MitoTracker. After incubation, the cells were washed with phosphate buffered saline (PBS) twice. The fluorescent signals of MitoTracker and Hoechst 33342 were detected using ZOE Fluorescent Cell Imager (Bio-Rad) or Cytation 5 Cell Imaging Multi-Mode Reader (BioTek). The signal intensities were quantified with ImageJ program.

Visualization of Thermogenesis: Adipose spheroids were incubated with 250 nM of ERthermAC temperature-sensitive live cell dye (Sigma-Aldrich, Cat. No. SCT057) in serum free medium for 30 min at 37° C. After incubation, the cells were washed with PBS twice. The fluorescent signals were detected using Cytation 5 Cell Imaging Multi-Mode Reader (BioTek) and signal intensities were quantified with ImageJ program.

Statistical Analysis: Statistical significance was analyzed using one-way ANOVA with a post-hoc Tukey test or a two-way ANOVA with a post-hoc Tukey test where applicable and assessed based on P value. The num-ber of values is listed for each analysis. All results with *P <0.05, **P<0.01, ***P<0.001, ****P<0.0001 were considered as statistically significant. The Statistical analysis was performed with Prism software version 10.1.0 (264).

### REFERENCES

[1] J. K. Lam, M. Y. Chow, Y. Zhang, S. W. Leung, Mol. Ther. Nucleic Acids 2015, 4, E252.
[2] D. Peer, J. Lieberman, Gene Ther. 2011, 18, 1127.
[3] Y. K. Kim, Exp. Mol. Med. 2022, 54, 455.
[4] A. Curreri, D. Sankholkar, S. Mitragotri, Z. Zhao, Bioeng. Transl. Med. 2022, 8, e10374.
[5] K. Tatiparti, S. Sau, S. K. Kashaw, A. K. Iyer, Nanomaterials 2017, 7, 77.
[6] A. L. Jackson, J. Burchard, D. Leake, A. Reynolds, J. Schelter, J. Guo, J. M. Johnson, L. Lim, J. Karpilow, K. Nichols, W. Marshall, A. Khvorova, P. S. Linsley, R N A 2006, 12, 1197.
[7] P. Wang, Y. Zhou, A. M. Richards, Theranostics 2021, 11, 8771.
[8] S. Kauppinen, B. Vester, J. Wengel, Drug Discov. Today Technol. 2005, 2, 287.
[9] M. S. Ebert, J. R. Neilson, P. A. Sharp, Nat. Methods 2007, 4, 721.
[10] Z. Wang, Methods Mol. Biol. 2011, 676, 43.
[11] J. A. Kulkarni, D. Witzigmann, S. B. Thomson, S. Chen, B. R. Leavitt, P. R. Cullis, R. van der Meel, Nat. Nanotechnol. 2021, 16, 630.
[12] F. Borel, M. A. Kay, C. Mueller, Mol. Ther. 2014, 22, 692.
[13] G. Calcaterra, P. P. Bassareo, F. Barilla, F. Romeo, J. L. Mehta, J. Car-diovasc. Med. (Hagerstown) 2021, 23, 71.
[14] J. G. Rizk, A. Gupta, P. Sardar, B. M. Henry, J. C. Lewin, G. Lippi, C. J. Lavie, JAMA Cardiol. 2021, 6, 1451.
[15] A. Greinacher, K. Selleng, J. Mayerle, R. Palankar, J. Wesche, S. Reiche, A. Aebischer, T. E. Warkentin, M. Muenchhoff, J. C. Hellmuth, O. T. Keppler, D. Duerschmied, A. Lother, S. Rieg, M. P. Gawaz, K. A. L. Mueller, C. S. Scheer, M. Napp, K. Hahnenkamp, G. Lucchese, A. Vogelgesang, A. Floel, P. Lovreglio, A. Stufano, R. Marschalek, T. Thiele, Blood 2021, 138, 1269.
[16] S. M. Moghimi, Mol. Ther. 2021, 29, 898.
[17] H. Jeon, S. Han, H. Kim, J. B. Lee, J. Ind. Eng. Chem. 2019, 70, 87.
[18] H. Kim, D. Kim, J. Jeong, H. Jeon, J. B. Lee, Polymers (Basel) 2018, 10, 589.
[19] M. Jang, J. H. Kim, H. Y. Nam, I. C. Kwon, H. J. Ahn, Nat. Commun. 2015, 6, 7930.
[20] J. B. Lee, J. Hong, D. K. Bonner, Z. Poon, P. T. Hammond, Nat. Mater. 2012, 11, 316.
[21] E. Kim, S. Agarwal, N. Kim, F. S. Hage, V. Leonardo, A. Gelmi, M. M. Stevens, ACS Nano 2019, 13, 2888.
[22] L. Meng, C. Liu, J. Lu, Q. Zhao, S. Deng, G. Wang, J. Qiao, C. Zhang, L. Zhen, Y. Lu, W. Li, Y. Zhang, R. G. Pestell, H. Fan, Y. i-H. Chen, Z. Liu, Z. Yu, Nat. Commun. 2017, 8, 13964.
[23] N. Arias, L. Aguirre, A. Fernández-Quintela, M. González, A. Lasa, J. Miranda, M. T. Macarulla, M. P. Portillo, J. Physiol. Biochem. 2016, 72, 509.
[24] M. Trajkovski, K. Ahmed, C. C. Esau, M. Stoffel, Nat. Cell Biol. 2012, 14, 1330.
[25] G. Solinas, J. Boren, A. G. Dulloo, Mol. Metab. 2015, 4, 367.
[26] S. Maurer, M. Harms, J. Boucher, FEBS J. 2021, 288, 3628.
[27] B. Cannon, J. Nedergaard, Physiol. Rev. 2004, 84, 277.
[28] P. Seale, B. Bjork, W. Yang, S. Kajimura, S. Chin, S. Kuang, A. Scimè, S. Devarakonda, H. M. Conroe, H. Erdjument-Bromage, P. Tempst, M. A. Rudnicki, D. R. Beier, B. M. Spiegelman, Nature 2008, 454, 961.
[29] S. Kajimura, P. Seale, K. Kubota, E. Lunsford, J. V. Frangioni, S. P. Gygi, B. M. Spiegelman, Nature 2009, 460, 1154.
[30] P. Seale, S. Kajimura, W. Yang, S. Chin, L. M. Rohas, M. Uldry, G. Tavernier, D. Langin, B. M. Spiegelman, Cell Metab. 2007, 6, 38.
[31] P. Seale, H. M. Conroe, J. Estall, S. Kajimura, A. Frontini, J. Ishibashi, P. Cohen, S. Cinti, B. M. Spiegelman, J. Clin. Invest. 2011, 121, 96.
[32] K. Ikeda, P. Maretich, S. Kajimura, Trends Endocrinol. Metab. 2018, 29, 191.
[33] W. Liu, P. Bi, T. Shan, X. Yang, H. Yin, Y. X. Wang, N. Liu, M. A. Rudnicki, S. Kuang, PLOS Genet. 2013, 9, e1003626.
[34] S. Kim, J. W. Park, M. G. Lee, K. H. Nam, J. H. Park, H. Oh, J. Lee, J. Han, S. A. Yi, J. W. Han, J. Cell. Physiol. 2019, 234, 3800.
[35] S. S. Choe, J. Y. Huh, I. J. Hwang, J. I. Kim, J. B. Kim, Front. Endocrinol. 2016, 7, 30.
[36] C. Hepler, R. K. Gupta, Mol. Cell. Endocrinol. 2017, 445, 95.
[37] S. Corvera, J. Solivan-Rivera, Z. Yang Loureiro, Angiogenesis 2022, 25, 439.
[38] C. M. Kusminski, P. E. Bickel, P. E. Scherer, Nat. Rev. Drug Discov. 2016, 15, 639.
[39] S. D. Rose, D. H. Kim, M. Amarzguioui, J. D. Heidel, M. A. Collingwood, M. E. Davis, J. J. Rossi, M. A. Behlke, Nucleic Acids Res. 2005, 33, 4140.
[40] S. A. Woodson, E. Koculi, Methods Enzymol. 2009, 469, 189.
[41] J. H. Lee, S. H. Ku, M. J. Kim, S. J. Lee, H. C. Kim, K. Kim, S. H. Kim, I. C. Kwon, J. Controlled Release 2017, 263, 29.
[42] H. Kim, J. Jeong, D. Kim, G. Kwak, S. H. Kim, J. B. Lee, Adv. Sci. 2017, 4, 1600523.
[43] D. Han, Y. Park, H. Kim, J. B. Lee, Nat. Commun. 2014, 5, 4367.
[44] D. Han, Y. Park, H. Nam, J. B. Lee, Chem. Commun. 2014, 50, 11665.
[45] M. H. Darvishi, A. Allahverdi, H. Hashemzadeh, H. R. Javadi, Sci. Rep. 2022, 12, 17520.
[46] J. Schauss, A. Kundu, B. P. Fingerhut, T. Elsaesser, J. Phys. Chem. B 2021, 125, 740.
[47] S. Chen, Q. Zhang, X. Wu, P. G. Schultz, S. Ding, J. Am. Chem. Soc. 2004, 126, 410.
[48] P. H. Hagedorn, R. Persson, E. D. Funder, N. Albæk, S. L. Diemer, D. J. Hansen, M. R. Møller, N. Papargyri, H. Christiansen, B. o R. Hansen, H. F. Hansen, M. A. Jensen, T. Koch, Drug Discov. Today 2018, 23, 101.
[49] T. C. Roberts, R. Langer, M. J. A Wood, Nat. Rev. Drug Discov. 2020, 19, 673.
[50] D. A. Braasch, D. R. Corey, Chem. Biol. 2001, 8, 1.
[51] J. Nedergaard, V. Golozoubova, A. Matthias, A. Asadi, A. Jacobsson, B. Cannon, Biochim. Biophys. Acta 2001, 1504, 82.
[52] M. Harms, P. Seale, Nat. Med. 2013, 19, 1252.
[53] A. J. Klingelhutz, F. A. Gourronc, A. Chaly, D. A. Wadkins, A. J. Burand, K. R. Markan, S. O. Idiga, M. Wu, M. J. Potthoff, J. A. Ankrum, Sci. Rep. 2018, 8, 523.
[54] A. D. Graham, R. Pandey, V. S. Tsancheva, A. Candeo, S. W. Botchway, A. J. Allan, L. Teboul, K. Madi, T. S. Babra, L. A. K. Zolkiewski, X. Xue, L. Bentley, J. Gannon, S. N. Olof, R. D. Cox, Biofabrication 2019, 12, 015018.
[55] A. Ioannidou, S. Alatar, R. Schipper, F. Baganha, M. Åhlander, A. Hornell, R. M. Fisher, C. E. Hagberg, J. Physiol. 2021, 600, 869.
[56] R. Sharma, S. Narum, S. Liu, Y. Dong, K. I. Baek, H. Jo, K. Salaita, ACS Chem. Biol. 2023, 18, 2349.
[57] J. Nedergaard, B. Cannon, Cell Metab. 2014, 20, 396.
[58] J. Grundlingh, P. I. Dargan, M. El-Zanfaly, J. Med. Toxicol. 2011, 7, 205.
[59] L. Tong, H. Xue, L. Xiong, J. Xiao, Y. Zhou, Mol. Biotechnol. 2015, 57, 939.
[60] C. Chen, Nucleic Acids Res. 2005, 33, e179.

### Example 2. Development of A2AR siRNA Nanoparticle and Chitosan Coating

Chitosan coating occurred with 100 uL of 0.2% low-molecular weight chitosan and 100 ug of nanoparticles. 100 uL of 0.2% chitosan and 100 ug of RNA NP were stirred in nuclease free water with 0.1% acetic acid for about 30 minutes to 4 hours at room temperature (1 hour is recommended) prior to centrifugation at 12,000 rpm for 30 minutes to purify the nanoparticles.

Adenosine is an endogenous neurotransmitter that plays an essential role in synaptic plasticity and maintenance of brain homeostasis through four distinct adenosine receptors: A₁R, A_{2A}R, A_{2B}R and A₃R. Of these, the G-protein coupled adenosine A2A receptor (A_{2A}R) is expressed abundantly in the striatum and to a lesser degree in the hippocampus, although it is well-known to be critical for hippocampal synaptic plasticity and memory function [3]. Importantly, human and mouse studies report increased levels of A_{2A}R expression in the hippocampus as a result of aging [4], Alzheimer's [5] and Parkinson's disease [3,6], clearly indicating the potential involvement of the A_{2A}R in neurodegenerative pathogenesis. Similarly, activating A_{2A}R signaling selectively within hippocampal neurons impairs synaptic plasticity and spatial memory function [4, 7]. In addition, pharmacologically increased A_{2A}R activation with the selective A_{2A}R agonist CGS 26180 also impairs recognition memory [8]. In contrast, selective inhibition of the A_{2A}R through antagonists such as istradefylline (KW-6002) or SCH58261 also enhances synaptic plasticity and spatial working memory in aging and Alzheimer's disease conditions in mice [9-12]. More importantly, clinical trials approved by the FDA reveal that istradefylline has proven to be safe,

well tolerated, and offers clinically meaningful recovery in Parkinson's disease [13]. Collectively, these observations strongly suggest that increased levels of the A_{2A}R can cause synaptic and cognitive impairments in neurodegenerative diseases. Therefore, A_{2A}R antagonists are the focus of active drug development for neurodegenerative diseases.

In addition to the importance of the A_{2A}R toward neurodegeneration, A_{2A}R antagonists are an emerging class of agents to treat cancer [18, 19]. Several studies have identified elevated amounts of extracellular adenosine in the tumor microenvironment as an important mechanism in cancer cell immune evasion, indicating A_{2A}R antagonism as an effective immuno-oncology therapy [20, 21]. Indeed, preclinical studies have demonstrated that A_{2A}R antagonists markedly enhance anti-tumor immunity, tumor vaccines, checkpoint blockade and adoptive T cell therapy [18, 19, 22, 23]. In addition, A_{2A}R inhibition such as caffeine or SCH58261 is shown to inhibit cancer growth in multiple cancers [23-25]. Moreover, A_{2A}R antagonists are currently undergoing clinical trials testing the safety, tolerability, and anti-tumor activity of the A_{2A}R antagonist CPI-444 as a single agent or in combination with PD-L1 inhibitors against various tumors including non-small cell lung cancer, and triple-negative breast [26, 27]. Collectively, A_{2A}R antagonists may represent the next generation of immune checkpoint inhibition in cancer immunotherapy.

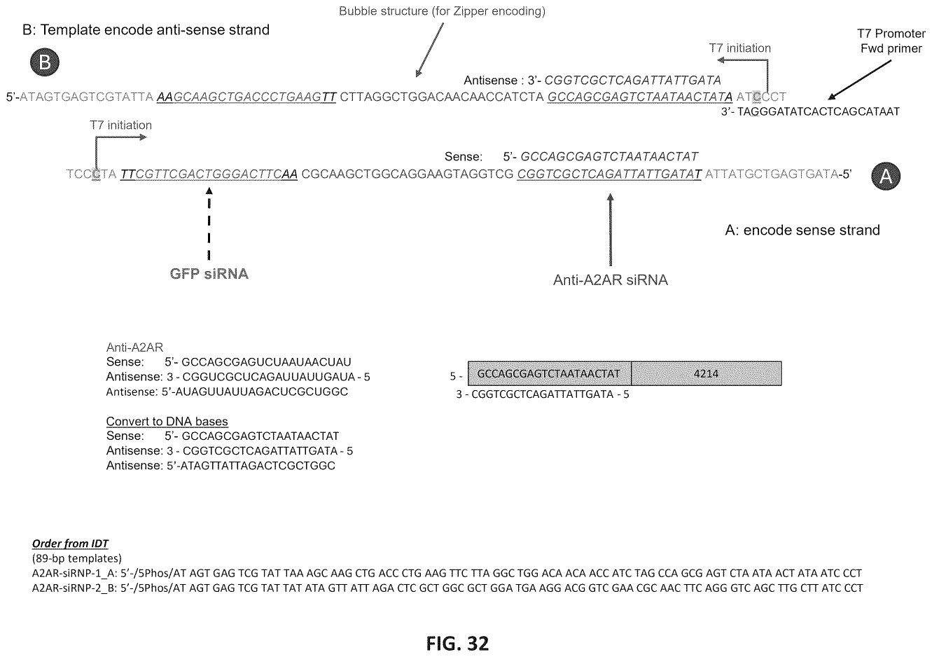

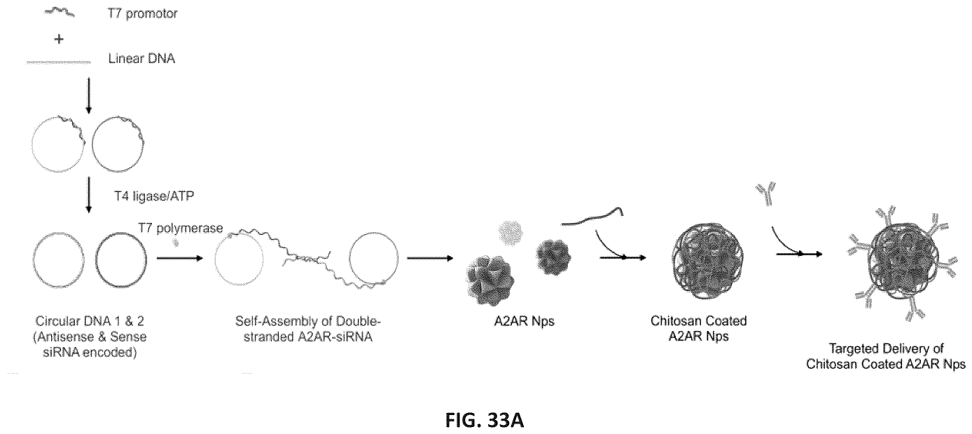

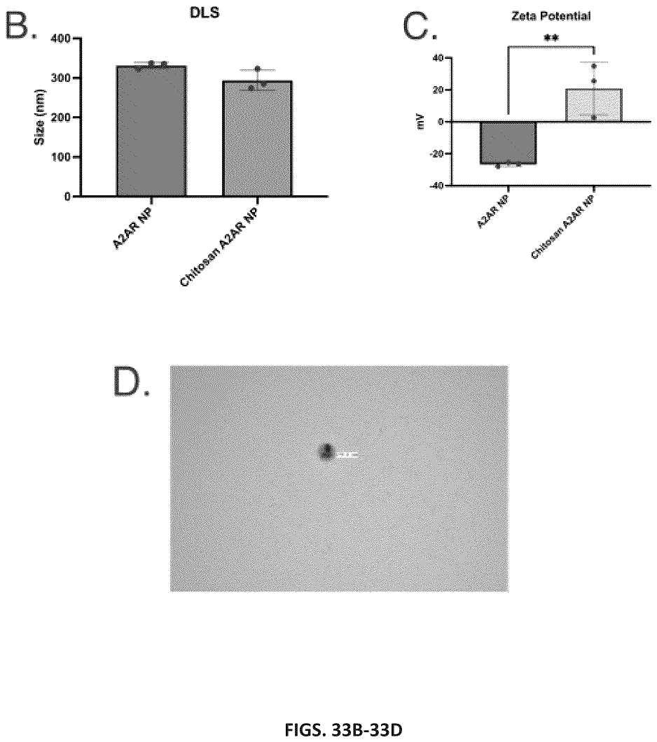

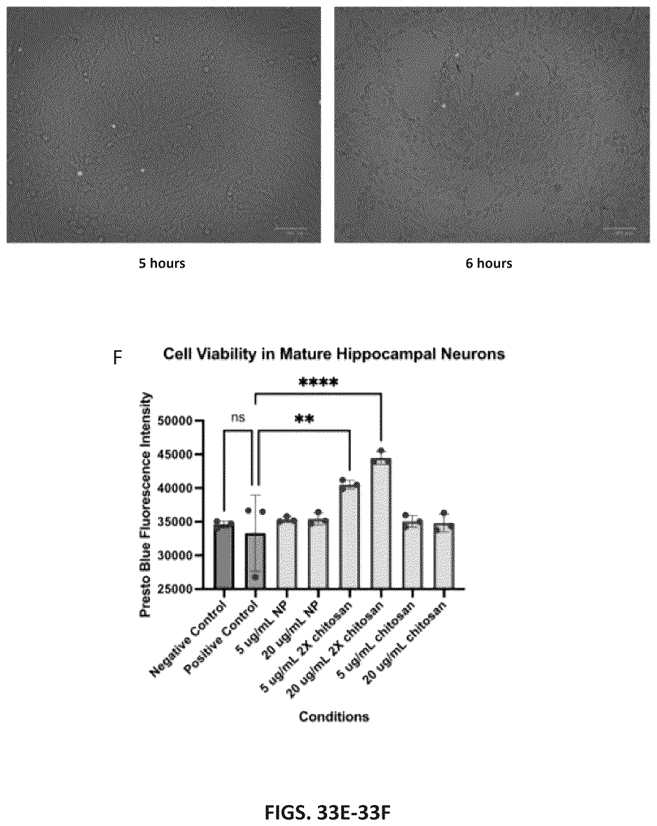

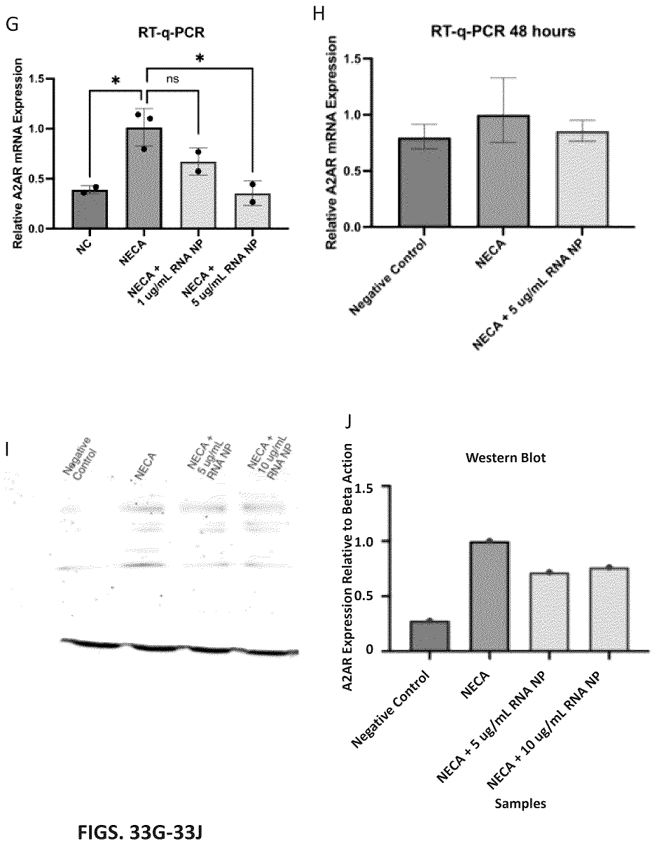

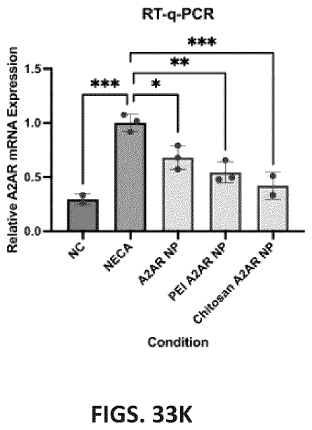

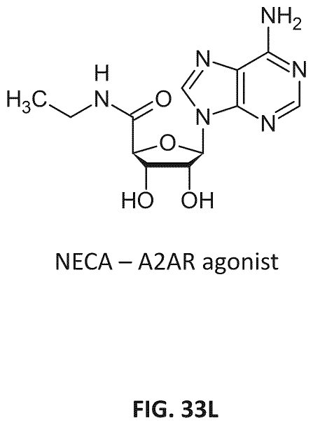

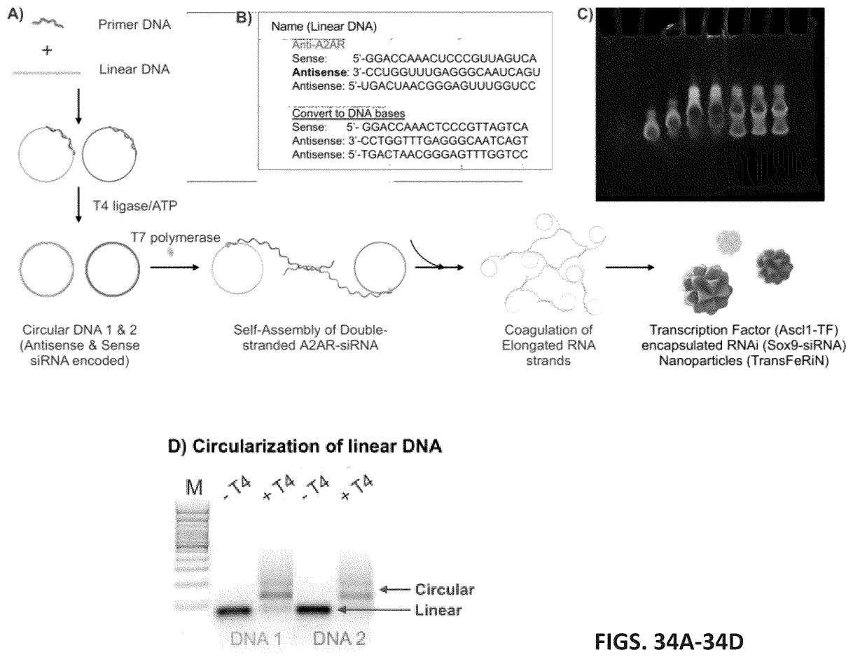

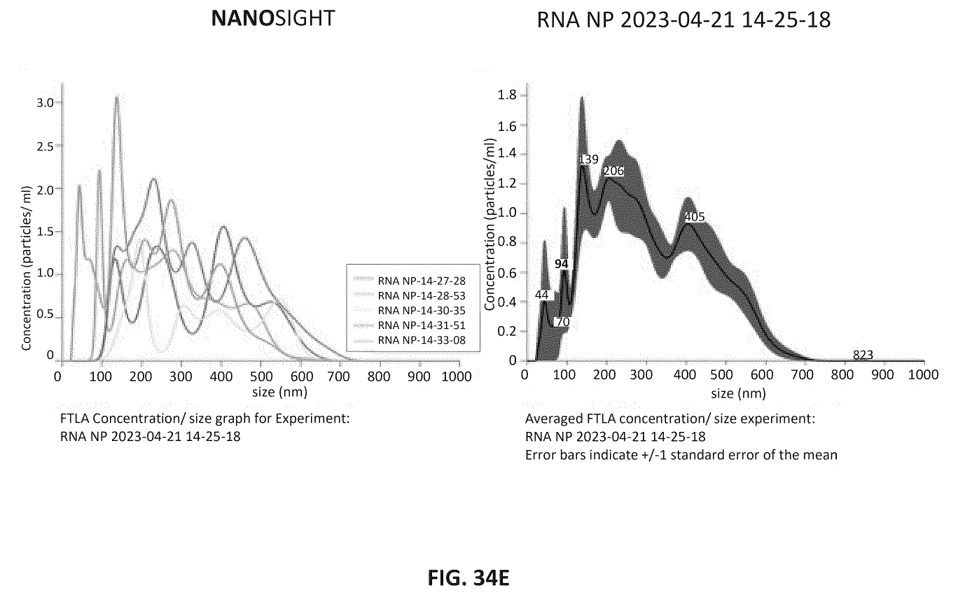

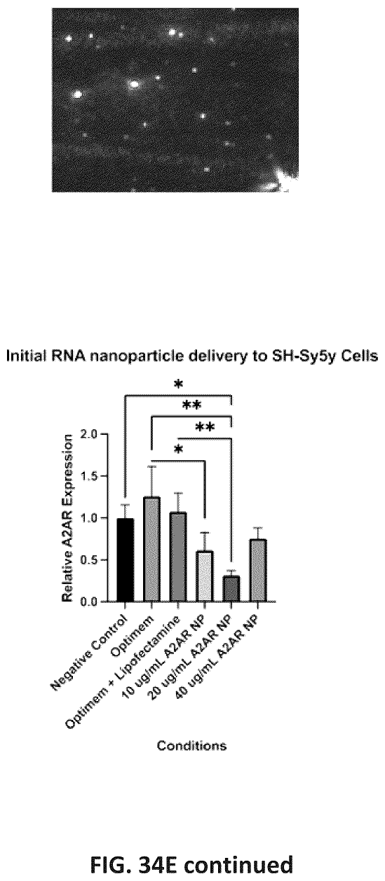

Here, we have developed a novel non-viral siRNA-A_{2A}R nanoparticle to achieve long-lasting A_{2A}R silencing in a cell-type-specific manner. The A_{2A}R RNA nanoparticles (FIG. 32) were synthesized and coated with chitosan, as shown in FIG. 33A. Chitosan, a biopolymer derived from chitin, was selected for its biodegradable properties and positive charge, facilitating the electrostatic coating of siRNA nanoparticles. Chitosan was chosen over polyethylenimine (PEI), an alternative coating material, due to its lower toxicity and enhanced biocompatibility. Various characterization techniques, including Dynamic Light Scattering (DLS), Zeta Potential analysis, and Transmission Electron Microscopy (TEM), confirmed the successful formation and chitosan coating of the nanoparticles. These analyses, depicted in FIGS. 33B-33D, also indicated that the chitosan-coated nanoparticles are suitable for future conjugation with antibodies or peptides to enable cell-specific targeting.

We incorporated Gel Red, an RNA and DNA stain, into the RNA nanoparticle system to optimize cellular uptake. The system was then purified to remove any free dye before delivering the chitosan-coated nanoparticles to mature hippocampal neurons and differentiated HT-22 cells, as illustrated in FIG. 33E. Cell viability assays were conducted under various conditions, with and without chitosan coating, revealing that none of the nanoparticle conditions adversely affected cell viability (FIG. 33F). To assess the effectiveness of the RNA nanoparticles in silencing A_{2A}R, RT-qPCR was performed at 24 and 48 hours post-delivery of bare A_{2A}R RNA nanoparticles. The results in FIGS. 33G and H confirmed a downregulation of A_{2A}R expression. Western blot analysis at 48 hours further demonstrated decreased levels of A_{2A}R protein (FIGS. 331 and J).

To compare the effectiveness of chitosan to PEI, we conducted RT-qPCR on HT-22 cells treated with bare RNA nanoparticles, PEI-coated RNA nanoparticles, and chitosan-coated RNA nanoparticles for 24 hours at a concentration of 5 μg/mL. The results indicated significant downregulation of A_{2A}R across all conditions, with the most pronounced effect observed following chitosan coating (FIG. 33K). This development of siRNA-A_{2A}R nanoparticles represents a significant step towards achieving prolonged and cell-type-specific silencing of A_{2A}R.

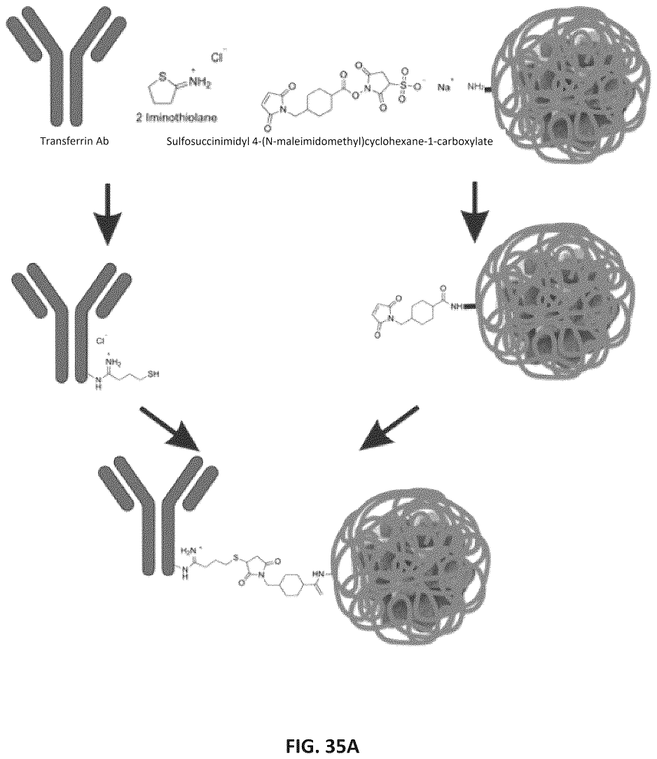

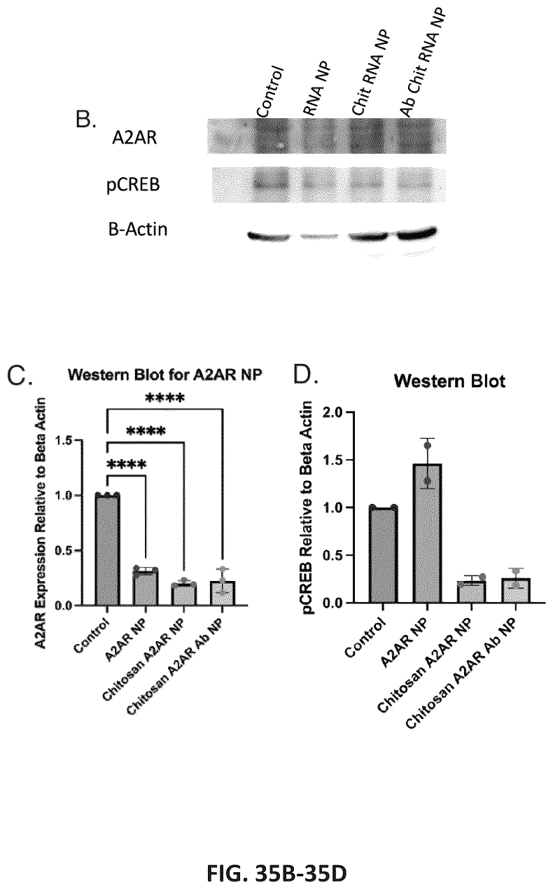

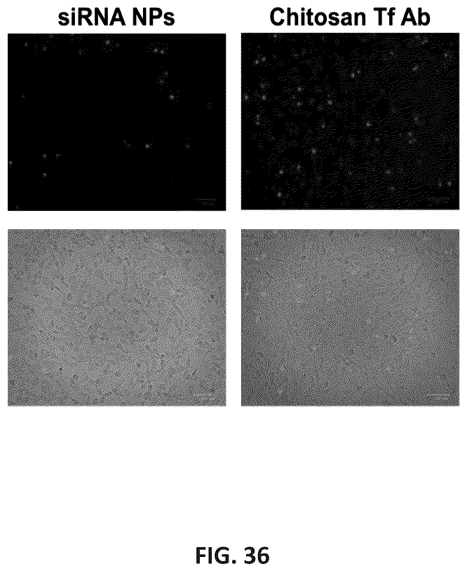

Antibody conjugation: Trout's reagent was added at equal molar ratio to the antibody of interest for thiol on each antibody (as shown in FIG. 35A) in buffer at neutral pH and shaken for at least 2 hours for conjugation. This was purified with a 10 kDa protein concentrator. Simultaneously, the RNA NP's were added with sulfo-SMCC in varying molar ratios for at least 3 hours. By adding more sulfa-SMCC, more of the nanoparticle surface can be covered with the antibodies. The RNA NPs are purified with centrifugation to pellet them and the supernatant is removed. Finally, the purified antibodies and RNA NPs are incubated together overnight for final antibody conjugation. The amount of sulfo-SMCC on the nanoparticle surface limits the amount of antibody that can attach to it. Therefore, the RNA NPs are purified one more time after incubation with the antibody by centrifuging to pellet them. Any free antibody is left in the supernatant and only the antibody-conjugated RNA NPs are left.

### REFERENCES

[1] C. Stavraka, A. Ford, S. Ghaem-Maghami, T. Crook, R. Agarwal, H. Gabra, S. Blagden, A study of symptoms described by ovarian cancer survivors, Gynecol Oncol, 125 (2012) 59-64.
[2] A. Oliveros, K. H. Yoo, M. A. Rashid, A. Corujo-Ramirez, B. Hur, J. Sung, Y. Liu, J. R. Hawse, D. S. Choi, D. Boison, M. H. Jang, Adenosine A2A receptor blockade prevents cisplatin-induced impairments in neurogenesis and cognitive function, Proc Natl Acad Sci USA, 119 (2022) e2206415119.
[3] J. F. Chen, Adenosine receptor control of cognition in normal and disease, Int Rev Neurobiol, 119 (2014) 257-307.
[4] M. Temido-Ferreira, D. G. Ferreira, V. L. Batalha, I. Marques-Morgado, J. E. Coelho, P. Pereira, R. Gomes, A. Pinto, S. Carvalho, P. M. Canas, L. Cuvelier, V. Buee-Scherrer, E. Faivre, Y. Baqi, C. E. Muller, J. Pimentel, S. N. Schiffmann, L. Buee, M. Bader, T. F. Outeiro, D. Blum, R. A. Cunha, H. Marie, P. A. Pousinha, L. V. Lopes, Age-related shift in LTD is dependent on neuronal adenosine A2A receptors interplay with mGluR5 and NMDA receptors, Mol Psychiatry, 25 (2020) 1876-1900.
[5] A. G. Orr, E. C. Hsiao, M. M. Wang, K. Ho, D. H. Kim, X. Wang, W. Guo, J. Kang, G. Q. Yu, A. Adame, N. Devidze, D. B. Dubal, E. Masliah, B. R. Conklin, L. Mucke, Astrocytic adenosine receptor A2A and Gs-coupled signaling regulate memory, Nat Neurosci, 18 (2015) 423-434.
[6] F. Calon, M. Dridi, O. Hornykiewicz, P. J. Bedard, A. H. Rajput, T. Di Paolo, Increased adenosine A2A receptors in the brain of Parkinson's disease patients with dyskinesias, Brain, 127 (2004) 1075-1084.
[7] P. Li, D. Rial, P. M. Canas, J. H. Yoo, W. Li, X. Zhou, Y. Wang, G. J. van Westen, M. P. Payen, E. Augusto, N. Goncalves, A. R. Tome, Z. Li, Z. Wu, X. Hou, Y. Zhou, P. I. A, E. S. Boyden, R. A. Cunha, J. Qu, J. F. Chen, Optogenetic activation of intracellular adenosine A2A receptor signaling in the hippocampus is sufficient to trigger CREB phosphorylation and impair memory, Mol Psychiatry, 20 (2015) 1481.
[8] N. Pagnussat, A. S. Almeida, D. M. Marques, F. Nunes, G. C. Chenet, P. H. Botton, S. Mioranzza, C. M. Loss, R. A. Cunha, L. O. Porciuncula, Adenosine A (2A) receptors are necessary and sufficient to trigger memory impairment in adult mice, Br J Pharmacol, 172 (2015) 3831-3845.
[9] Z. Li, X. Chen, T. Wang, Y. Gao, F. Li, L. Chen, J. Xue, Y. He, Y. Li, W. Guo, W. Zheng, L. Zhang, F. Ye, X. Ren, Y. Feng, P. Chan, J. F. Chen, The Corticostriatal Adenosine A2A Receptor Controls Maintenance and Retrieval of Spatial Working Memory, Biol Psychiatry, 83 (2018) 530-541.
[10] A. C. Silva, C. Lemos, F. Q. Goncalves, A. V. Pliassova, N.J. Machado, H. B. Silva, P. M. Canas, R. A. Cunha, J. P. Lopes, P. Agostinho, Blockade of adenosine A2A receptors recovers early deficits of memory and plasticity in the triple transgenic mouse model of Alzheimer's disease, Neurobiol Dis, 117 (2018) 72-81.
[11] A. G. Orr, I. Lo, H. Schumacher, K. Ho, M. Gill, W. Guo, D. H. Kim, A. Knox, T. Saito, T. C. Saido, J. Simms, C. Toddes, X. Wang, G. Q. Yu, L. Mucke, Istradefylline reduces memory deficits in aging mice with amyloid pathology, Neurobiol Dis, 110 (2018) 29-36.
[12] S. V. da Silva, M. G. Haberl, P. Zhang, P. Bethge, C. Lemos, N. Goncalves, A. Gorlewicz, M. Malezieux, F. Q. Goncalves, N. Grosjean, C. Blanchet, A. Frick, U. V. Nagerl, R. A. Cunha, C. Mulle, Early synaptic deficits in the APP/PS1 mouse model of Alzheimer's disease involve neuronal adenosine A (2A) receptors, Nature Communications, 7 (2016).
[13] P. A. LeWitt, M. Guttman, J. W. Tetrud, P. J. Tuite, A. Mori, P. Chaikin, N. M. Sussman, U.S.S. Group, Adenosine A2A receptor antagonist istradefylline (KW-6002) reduces “off” time in Parkinson's disease: a double-blind, randomized, multicenter clinical trial (6002-US-005), Ann Neurol, 63 (2008) 295-302.
[14] K. H. Yoo, J. J. Tang, M. A. Rashid, C. H. Cho, A. Corujo-Ramirez, J. Choi, M. G. Bae, D. Brogren, J. R. Hawse, X. Hou, S. J. Weroha, A. Oliveros, L. A. Kirkeby, J. A. Baur, M. H. Jang, Nicotinamide Mononucleotide Prevents Cisplatin-Induced Cognitive Impairments, Cancer Res, 81 (2021) 3727-3737.
[15] L. D. Nguyen, T. T. Fischer, B. E. Ehrlich, Pharmacological rescue of cognitive function in a mouse model of chemobrain, Mol Neurodegener, 16 (2021) 41.
[16] B. E. Lee, B. Y. Choi, D. K. Hong, J. H. Kim, S. H. Lee, A. R. Kho, H. Kim, H. C. Choi, S. W. Suh, The cancer chemotherapeutic agent paclitaxel (Taxol) reduces hippocampal neurogenesis via down-regulation of vesicular zinc, Sci Rep, 7 (2017) 11667.
[17] P. Huehnchen, W. Boehmerle, A. Springer, D. Freyer, M. Endres, A novel preventive therapy for paclitaxel-induced cognitive deficits: preclinical evidence from C57BL/6 mice, Transl Psychiatry, 7 (2017) e1185.
[18] R. D. Leone, Y. C. Lo, J. D. Powell, A2aR antagonists: Next generation checkpoint blockade for cancer immunotherapy, Comput Struct Biotechnol J, 13 (2015) 265-272.
[19] M. Congreve, G. A. Brown, A. Borodovsky, M. L. Lamb, Targeting adenosine A2A receptor antagonism for treatment of cancer, Expert Opin Drug Discov, 13 (2018) 997-1003.
[20] M. Mediavilla-Varela, K. Luddy, D. Noyes, F. K. Khalil, A. M. Neuger, H. Soliman, S. J. Antonia, Antagonism of adenosine A2A receptor expressed by lung adenocarcinoma tumor cells and cancer associated fibroblasts inhibits their growth, Cancer Biol Ther, 14 (2013) 860-868.
[21] N. Etique, I. Grillier-Vuissoz, J. Lecomte, S. Flament, Crosstalk between adenosine receptor (A2A isoform) and ERalpha mediates ethanol action in MCF-7 breast cancer cells, Oncol Rep, 21 (2009) 977-981.
[22] S. B. Willingham, P. Y. Ho, A. Hotson, C. Hill, E. C. Piccione, J. Hsieh, L. Liu, J. J. Buggy, I. McCaffery, R. A. Miller, A_{2A}R Antagonism with CPI-444 Induces Antitumor Responses and Augments Efficacy to Anti-PD-(L) 1 and Anti-CTLA-4 in Preclinical Models, Cancer Immunol Res, 6 (2018) 1136-1149.
[23] A. Ohta, E. Gorelik, S. J. Prasad, F. Ronchese, D. Lukashev, M. K. Wong, X. Huang, S. Caldwell, K. Liu, P. Smith, J. F. Chen, E. K. Jackson, S. Apasov, S. Abrams, M. Sitkovsky, A2A adenosine receptor protects tumors from antitumor T cells, Proc Natl Acad Sci USA, 103 (2006) 13132-13137.
[24] P. A. Beavis, U. Divisekera, C. Paget, M. T. Chow, L. B. John, C. Devaud, K. Dwyer, J. Stagg, M. J. Smyth, P. K. Darcy, Blockade of A2A receptors potently suppresses the metastasis of CD73+ tumors, Proc Natl Acad Sci USA, 110 (2013) 14711-14716.
[25] Z. Wang, C. Gu, X. Wang, Y. Lang, Y. Wu, X. Wu, X. Zhu, K. Wang, H. Yang, Caffeine enhances the anti-tumor effect of 5-fluorouracil via increasing the production of reactive oxygen species in hepatocellular carcinoma, Med Oncol, 36 (2019) 97.
[26] S. B. Willingham, A. N. Hotson, R. A. Miller, Targeting the A_{2A}R in cancer; early lessons from the clinic, Curr Opin Pharmacol, 53 (2020) 126-133.
[27] C. Sun, B. Wang, S. Hao, Adenosine-A2A Receptor Pathway in Cancer Immunotherapy, Front Immunol, 13 (2022) 837230.
[28] J. Wen, R. R. Maxwell, A. J. Wolf, M. Spira, M. E. Gulinello, P. D. Cole, Methotrexate causes persistent deficits in memory and executive function in a juvenile animal model, Neuropharmacology, 139 (2018) 76-84.
[29] A. M. Williams, P. D. Cole, Biomarkers of Cognitive Impairment in Pediatric Cancer Survivors, J Clin Oncol, 39 (2021) 1766-1774.
[30] D. Han, Y. Park, H. Kim, J. B. Lee, Self-assembly of free-standing RNA membranes, Nat Commun, 5 (2014) 4367.
[31] S. Han, H. Kim, J. B. Lee, Library siRNA-generating RNA nanosponges for gene silencing by complementary rolling circle transcription, Sci Rep, 7 (2017) 10005.
[32] S. Shah, A. Solanki, P. K. Sasmal, K. B. Lee, Single vehicular delivery of siRNA and small molecules to control stem cell differentiation, J Am Chem Soc, 135 (2013) 15682-15685.
[33] Y. Nieto, P. J. Cagnoni, S. I. Bearman, E. J. Shpall, S. Matthes, T. DeBoom, A. Baron, R. B. Jones, Acute encephalopathy: a new toxicity associated with high-dose paclitaxel, Clin Cancer Res, 5 (1999) 501-506.
[34] S. Muallaoglu, M. Kocer, N. Guler, Acute transient encephalopathy after weekly paclitaxel infusion, Med Oncol, 29 (2012) 1297-1299.
[35] C. G. Ziske, B. Schottker, M. Gorschluter, U. Mey, R. Kleinschmidt, U. Schlegel, T. Sauerbruch, I. G. Schmidt-Wolf, Acute transient encephalopathy after paclitaxel infusion: report of three cases, Ann Oncol, 13 (2002) 629-631.
[36] N. P. Staff, A. Grisold, W. Grisold, A. J. Windebank, Chemotherapy-induced peripheral neuropathy: A current review, Ann Neurol, 81 (2017) 772-781.
[37] M. Chovanec, L. Vasilkova, L. Setteyova, J. Obertova, P. Palacka, K. Rejlekova, Z. Sycova-Mila, K. Kalavska, D. Svetlovska, S. Cingelova, B. Mladosievicova, J. Mardiak, M. Mego, Long-Term Cognitive Functioning in Testicular Germ-Cell Tumor Survivors, Oncologist, 23 (2018) 617-623.
[38] C. D. Scripture, W. D. Figg, A. Sparreboom, Peripheral neuropathy induced by paclitaxel: recent insights and future perspectives, Curr Neuropharmacol, 4 (2006) 165-172.
[39] S. Dutta, P. Sengupta, Men and mice: Relating their ages, Life Sci, 152 (2016) 244-248.
[40] S. Manohar, S. Jamesdaniel, R. Salvi, Cisplatin inhibits hippocampal cell proliferation and alters the expression of apoptotic genes, Neurotox Res, 25 (2014) 369-380.
[41] W. Zhou, A. Kavelaars, C. J. Heijnen, Metformin Prevents Cisplatin-Induced Cognitive Impairment and Brain Damage in Mice, PLOS One, 11 (2016) e0151890.
[42] X. Le, E. Y. Hanna, Optimal regimen of cisplatin in squamous cell carcinoma of head and neck yet to be determined, Ann Transl Med, 6 (2018) 229.
[43] P. Szturz, K. Wouters, N. Kiyota, M. Tahara, K. Prabhash, V. Noronha, D. Adelstein, D. Van Gestel, J. B. Vermorken, Low-Dose vs. High-Dose Cisplatin: Lessons Learned From 59 Chemoradiotherapy Trials in Head and Neck Cancer, Front Oncol, 9 (2019) 86.
[44] G. Cavaletti, L. Marzorati, G. Bogliun, N. Colombo, M. Marzola, M. R. Pittelli, G. Tredici, Cisplatin-induced peripheral neurotoxicity is dependent on total-dose intensity and single-dose intensity, Cancer, 69 (1992) 203-207.
[45] R. G. van der Hoop, M. E. van der Burg, W. W. ten Bokkel Huinink, C. van Houwelingen, J. P. Neijt, Incidence of neuropathy in 395 patients with ovarian cancer treated with or without cisplatin, Cancer, 66 (1990) 1697-1702.
[46] D. P. Waber, G. Gioia, J. Paccia, B. Sherman, D. Dinklage, N. Sollee, D. K. Urion, N.J. Tarbell, S. E. Sallan, Sex differences in cognitive processing in children treated with CNS prophylaxis for acute lymphoblastic leukemia, J Pediatr Psychol, 15 (1990) 105-122.
[47] E. van der Plas, W. Qiu, B. J. Nieman, Y. Yasui, Q. Liu, S. B. Dixon, N. S. Kadan-Lottick, C. B. Weldon, B. R. Weil, L. M. Jacola, T. M. Gibson, W. Leisenring, K. Oeffinger, M. M. Hudson, L. L. Robison, G. T. Armstrong, K. R. Krull, Sex-Specific Associations Between Chemotherapy, Chronic Conditions, and Neurocognitive Impairment in Acute Lymphoblastic Leukemia Survivors: A Report From the Childhood Cancer Survivor Study, J Natl Cancer Inst, 113 (2021) 588-596.
[48] M. H. Jang, M. A. Bonaguidi, Y. Kitabatake, J. Sun, J. Song, E. Kang, H. Jun, C. Zhong, Y. Su, J. U. Guo, M. X. Wang, K. A. Sailor, J. Y. Kim, Y. Gao, K. M. Christian, G. L. Ming, H. Song, Secreted frizzled-related protein 3 regulates activity-dependent adult hippocampal neurogenesis, Cell Stem Cell, 12 (2013) 215-223.
[49] M. H. Jang, Y. Kitabatake, E. Kang, H. Jun, M. V. Pletnikov, K. M. Christian, R. Hen, S. Lucae, E. B. Binder, H. Song, G. I. Ming, Secreted frizzled-related protein 3 (sFRP3) regulates antidepressant responses in mice and humans, Mol Psychiatry, (2012).
[50] S. M. Hussaini, H. Jun, C. H. Cho, H. J. Kim, W. R. Kim, M. H. Jang, Heat-induced antigen retrieval: an effective method to detect and identify progenitor cell types during adult hippocampal neurogenesis, J Vis Exp, (2013).
[51] C. E. Terrillion, B. Abazyan, Z. Yang, J. Crawford, A. V. Shevelkin, Y. Jouroukhin, K. H. Yoo, C. H. Cho, R. Roychaudhuri, S. H. Snyder, M. H. Jang, M. V. Pletnikov, DISC1 in Astrocytes Influences Adult Neurogenesis and Hippocampus-Dependent Behaviors in Mice, Neuropsychopharmacology, 42 (2017) 2242-2251.
[52] D. G. Ferreira, M. Temido-Ferreira, H. Vicente Miranda, V. L. Batalha, J. E. Coelho, E. M. Szego, I. Marques-Morgado, S. H. Vaz, J. S. Rhee, M. Schmitz, I. Zerr, L. V. Lopes, T. F. Outeiro, alpha-synuclein interacts with PrP(C) to induce cognitive impairment through mGluR5 and NMDAR2B, Nat Neurosci, 20 (2017) 1569-1579.
[53] W. Li, H. B. Silva, J. Real, Y. M. Wang, D. Rial, P. Li, M. P. Payen, Y. Zhou, C. E. Muller, A. R. Tome, R. A. Cunha, J. F. Chen, Inactivation of adenosine A2A receptors reverses working memory deficits at early stages of Huntington's disease models, Neurobiol Dis, 79 (2015) 70-80.
[54] T. Muller, The safety of istradefylline for the treatment of Parkinson's disease, Expert Opin Drug Saf, 14 (2015) 769-775.
[55] F. M. Mouro, A. Kofalvi, L. A. Andre, Y. Baqi, C. E. Muller, J. A. Ribeiro, A. M. Sebastiao, Memory deficits induced by chronic cannabinoid exposure are prevented by adenosine A2AR receptor antagonism, Neuropharmacology, 155 (2019) 10-21.
[56] M. P. Kaster, N.J. Machado, H. B. Silva, A. Nunes, A. P. Ardais, M. Santana, Y. Baqi, C. E. Muller, A. L. Rodrigues, L. O. Porciuncula, J. F. Chen, A. R. Tome, P. Agostinho, P. M. Canas, R. A. Cunha, Caffeine acts through neuronal adenosine A2A receptors to prevent mood and memory dysfunction triggered by chronic stress, Proc Natl Acad Sci USA, 112 (2015) 7833-7838.
[57] M. H. Jang, Y. Kitabatake, E. Kang, H. Jun, M. V. Pletnikov, K. M. Christian, R. Hen, S. Lucae, E. B. Binder, H. Song, G. I. Ming, Secreted frizzled-related protein 3 (sFRP3) regulates antidepressant responses in mice and humans, Mol Psychiatry, 18 (2013) 957-958.
[58] C. I. Choi, K. H. Yoo, S. M. Hussaini, B. T. Jeon, J. Welby, H. Gan, I. A. Scarisbrick, Z. Zhang, D. J. Baker, J. M. van Deursen, M. Rodriguez, M. H. Jang, The progeroid gene BubRI regulates axon myelination and motor function, Aging (Albany NY), 8 (2016) 2667-2688.
[59] A. R. Preston, H. Eichenbaum, Interplay of hippocampus and prefrontal cortex in memory, Curr Biol, 23 (2013) R764-773.
[60] J. A. Dumas, J. Makarewicz, G. J. Schaubhut, R. Devins, K. Albert, K. Dittus, P. A. Newhouse, Chemotherapy altered brain functional connectivity in women with breast cancer: a pilot study, Brain Imaging Behav, 7 (2013) 524-532.
[61] S. R. Kesler, J. S. Kent, R. O'Hara, Prefrontal cortex and executive function impairments in primary breast cancer, Arch Neurol, 68 (2011) 1447-1453.
[62] H. Cheng, W. Li, L. Gong, H. Xuan, Z. Huang, H. Zhao, L. S. Wang, K. Wang, Altered resting-state hippocampal functional networks associated with chemotherapy-induced prospective memory impairment in breast cancer survivors, Sci Rep, 7 (2017) 45135.
[63] S. Kesler, M. Janelsins, D. Koovakkattu, O. Palesh, K. Mustian, G. Morrow, F. S. Dhabhar, Reduced hippocampal volume and verbal memory performance associated with interleukin-6 and tumor necrosis factor-alpha levels in chemotherapy-treated breast cancer survivors, Brain Behav Immun, 30 Suppl (2013) S109-116.
[64] L. E. Ta, P. A. Low, A. J. Windebank, Mice with cisplatin and oxaliplatin-induced painful neuropathy develop distinct early responses to thermal stimuli, Mol Pain, 5 (2009) 9.
[65] Z. A. Zhao, Y. Zhao, Y. L. Ning, N. Yang, Y. Peng, P. Li, X. Y. Chen, D. Liu, H. Wang, X. Chen, W. Bai, J. F. Chen, Y. G. Zhou, Adenosine A2A receptor inactivation alleviates early-onset cognitive dysfunction after traumatic brain injury involving an inhibition of tau hyperphosphorylation, Transl Psychiatry, 7 (2017) e1123.
[66] V. Shah, O. Taratula, O. B. Garbuzenko, O. R. Taratula, L. Rodriguez-Rodriguez, T. Minko, Targeted nanomedicine for suppression of CD44 and simultaneous cell death induction in ovarian cancer: an optimal delivery of siRNA and anticancer drug, Clin Cancer Res, 19 (2013) 6193-6204.
[67] P. T. Yin, S. Shah, N.J. Pasquale, O. B. Garbuzenko, T. Minko, K. B. Lee, Stem cell-based gene therapy activated using magnetic hyperthermia to enhance the treatment of cancer, Biomaterials, 81 (2016) 46-57.
[68] A. G. Orr, A. L. Orr, X. J. Li, R. E. Gross, S. F. Traynelis, Adenosine A (2A) receptor mediates microglial process retraction, Nat Neurosci, 12 (2009) 872-878.
[69] D. G. Amaral, H. E. Scharfman, P. Lavenex, The dentate gyrus: fundamental neuroanatomical organization (dentate gyrus for dummies), Prog Brain Res, 163 (2007) 3-22.
[70] C. J. Wei, P. Singer, J. Coelho, D. Boison, J. Feldon, B. K. Yee, J. F. Chen, Selective inactivation of adenosine A (2A) receptors in striatal neurons enhances working memory and reversal learning, Learn Mem, 18 (2011) 459-474.
[71] X. Wang, C. Zhang, G. Szabo, Q. Q. Sun, Distribution of CaMKIIalpha expression in the brain in vivo, studied by CaMKIIalpha-GFP mice, Brain Res, 1518 (2013) 9-25.
[72] L. Madisen, T. A. Zwingman, S. M. Sunkin, S. W. Oh, H. A. Zariwala, H. Gu, L. L. Ng, R. D. Palmiter, M. J. Hawrylycz, A. R. Jones, E. S. Lein, H. Zeng, A robust and high-throughput Cre reporting and characterization system for the whole mouse brain, Nat Neurosci, 13 (2010) 133-140.
[73] P. Rotheneichner, P. Romanelli, L. Bieler, S. Pagitsch, P. Zaunmair, C. Kreutzer, R. Konig, J. Marschallinger, L. Aigner, S. Couillard-Despres, Tamoxifen Activation of Cre-Recombinase Has No Persisting Effects on Adult Neurogenesis or Learning and Anxiety, Front Neurosci, 11 (2017) 27.
[74] Y. Naito, K. Ui-Tei, siRNA Design Software for a Target Gene-Specific RNA Interference, Front Genet, 3 (2012) 102.
[75] P. T. Yin, T. Pongkulapa, H. Y. Cho, J. Han, N.J. Pasquale, H. Rabie, J. H. Kim, J. W. Choi, K. B. Lee, Overcoming Chemoresistance in Cancer via Combined MicroRNA Therapeutics with Anticancer Drugs Using Multifunctional Magnetic Core-Shell Nanoparticles, ACS Appl Mater Interfaces, 10 (2018) 26954-26963.
[76] C. Kim, B. P. Shah, P. Subramaniam, K. B. Lee, Synergistic induction of apoptosis in brain cancer cells by targeted codelivery of siRNA and anticancer drugs, Mol Pharm, 8 (2011) 1955-1961.
[77] Y. B. Seven, R. R. Perim, O. R. Hobson, A. K. Simon, A. Tadjalli, G. S. Mitchell, Phrenic motor neuron adenosine 2A receptors elicit phrenic motor facilitation, J Physiol, 596 (2018) 1501-1512.
[78] Z. Liu, S. Yan, J. Wang, Y. Xu, Y. Wang, S. Zhang, X. Xu, Q. Yang, X. Zeng, Y. Zhou, X. Gu, S. Lu, Z. Fu, D. J. Fulton, N. L. Weintraub, R. B. Caldwell, W. Zhang, C. Wu, X. L. Liu, J. F. Chen, A. Ahmad, I. Kaddour-Djebbar, M. Al-Shabrawey, Q. Li, X. Jiang, Y. Sun, A. Sodhi, L. Smith, M. Hong, Y. Huo, Endothelial adenosine A2a receptor-mediated glycolysis is essential for pathological retinal angiogenesis, Nat Commun, 8 (2017) 584.
[79] C. Helmschrodt, S. Hobel, S. Schoniger, A. Bauer, J. Bonicelli, M. Gringmuth, S. A. Fietz, A. Aigner, A. Richter, F. Richter, Polyethylenimine Nanoparticle-Mediated siRNA Delivery to Reduce alpha-Synuclein Expression in a Model of Parkinson's Disease, Mol Ther Nucleic Acids, 9 (2017) 57-68.
[80] H. Lee, A. K. Lytton-Jean, Y. Chen, K. T. Love, A. I. Park, E. D. Karagiannis, A. Sehgal, W. Querbes, C. S. Zurenko, M. Jayaraman, C. G. Peng, K. Charisse, A. Borodovsky, M. Manoharan, J. S. Donahoe, J. Truelove, M. Nahrendorf, R. Langer, D. G. Anderson, Molecularly self-assembled nucleic acid nanoparticles for targeted in vivo siRNA delivery, Nat Nanotechnol, 7 (2012) 389-393.
[81] B. Shah, P. T. Yin, S. Ghoshal, K.-B. Lee, Multimodal Magnetic Core-Shell Nanoparticles for Effective Stem-Cell Differentiation and Imaging, Angewandte Chemie International Edition, 52 (2013) 6190-6195.
[82] S. Shah, A. Solanki, P. K. Sasmal, K.-B. Lee, Single Vehicular Delivery of siRNA and Small Molecules to Control Stem Cell Differentiation, Journal of the American Chemical Society, 135 (2013) 15682-15685.
[83] A. Solanki, S. Shah, P. T. Yin, K.-B. Lee, Nanotopography-mediated Reverse Uptake for siRNA Delivery into Neural Stem Cells to Enhance Neuronal Differentiation, Scientific Reports, 3 (2013) 1553.
[84] P. T. Yin, T. Pongkulapa, H.-Y. Cho, J. Han, N.J. Pasquale, H. Rabie, J.-H. Kim, J.-W. Choi, K.-B. Lee, Overcoming Chemoresistance in Cancer via Combined MicroRNA Therapeutics with Anticancer Drugs Using Multifunctional Magnetic Core-Shell Nanoparticles, ACS Applied Materials & Interfaces, 10 (2018) 26954-26963.
[85] P. T. Yin, B. P. Shah, K.-B. Lee, Combined Magnetic Nanoparticle-based MicroRNA and Hyperthermia Therapy to Enhance Apoptosis in Brain Cancer Cells, Small, 10 (2014) 4106-4112.
[86] C. Helmschrodt, S. Höbel, S. Schöniger, A. Bauer, J. Bonicelli, M. Gringmuth, S. A. Fietz, A. Aigner, A. Richter, F. Richter, Polyethylenimine Nanoparticle-Mediated siRNA Delivery to Reduce α-Synuclein Expression in a Model of Parkinson's Disease, Molecular Therapy-Nucleic Acids, 9 (2017) 57-68.
[87] W. Gao, J. Li, Targeted siRNA delivery reduces nitric oxide mediated cell death after spinal cord injury, Journal of Nanobiotechnology, 15 (2017) 38.
[88] L. Yang, T.-H. Kim, H.-Y. Cho, J. Luo, J.-M. Lee, S.-T. D. Chueng, Y. Hou, P. T.-T. Yin, J. Han, J. H. Kim, B. G. Chung, J.-W. Choi, K.-B. Lee, Hybrid Graphene-Gold Nanoparticle-Based Nucleic Acid Conjugates for Cancer-Specific Multimodal Imaging and Combined Therapeutics, Advanced Functional Materials, 31 (2021) 2006918.
[89] P. T. Yin, S. Shah, N.J. Pasquale, O. B. Garbuzenko, T. Minko, K.-B. Lee, Stem cell-based gene therapy activated using magnetic hyperthermia to enhance the treatment of cancer, Biomaterials, 81 (2016) 46-57.

### Example 3. Development of PDE2A siRNA Nanoparticle and Lipid Coating

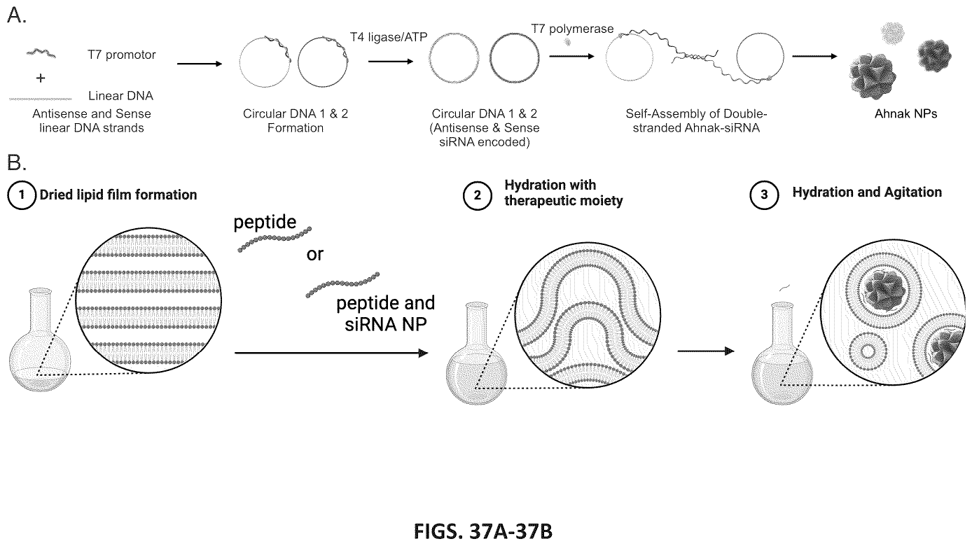

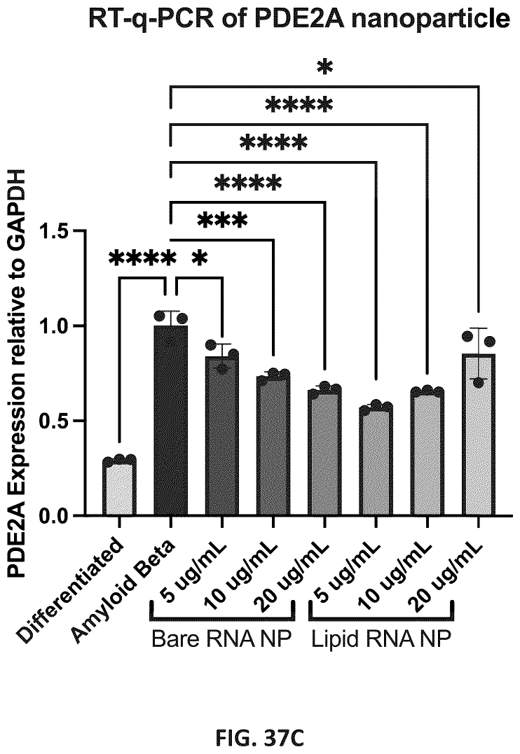

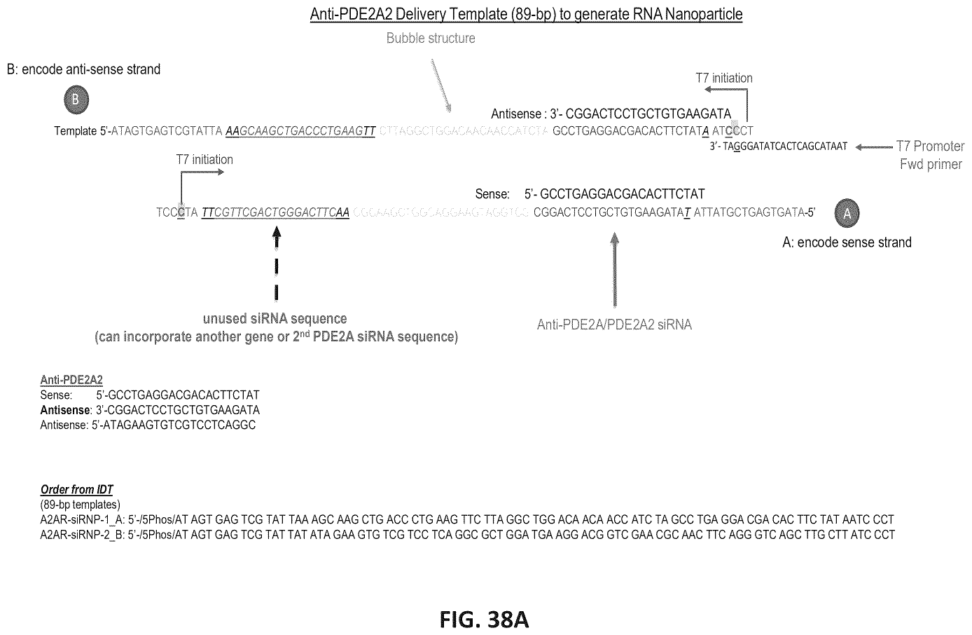

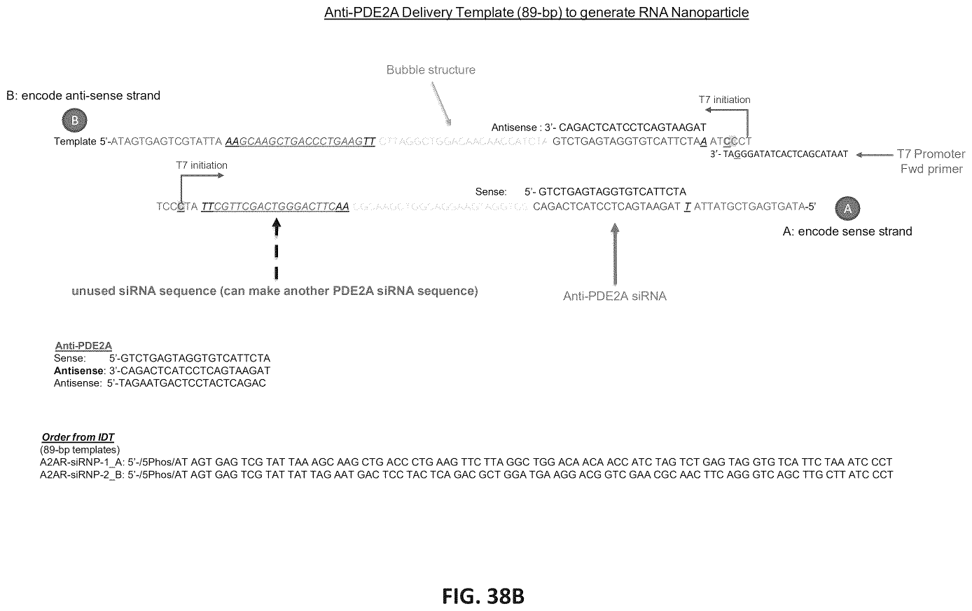

A PDE2A RNA nanoparticle was designed through methodological design of PDE2A siRNA for efficient silencing effect, cDNA formation from the RNA templates (FIG. 38A-38B), and finally RNA NP formation with T7 polymerase (FIG. 37A) Lipids are put together in varying ratios for positively charged liposome/lipid nanoparticle formation around the RNA NP (FIG. 37B). Thin film hydration is a commonly utilized method for encapsulating polar substrates. Briefly, we add phosphatidylcholine and cholesterol at a 7:3 ratio to chloroform in a round bottom flask. Removing the solvents under rotary evaporation forms a thin film. The purified siRNA is added to the thin film and sonicated for approximately 30 minutes to achieve liposomes of ˜200 nm. Encapsulated nanoparticles are purified by centrifuging at 10,000 rpm for 10 min. The liposomes are doped with DSPE-PEG-maleimide lipid at a 1% molar ratio through the technique of post-insertion. The thiol of a targeting moiety spontaneously forms a covalent bond with the maleimide of the lipid on the nanoparticle surface. RT-q-PCR showed that less RNA NP was required for PDE2A downregulation with lipid coating than bare nanoparticle delivered alone (FIG. 37C). PDE2A-targeting nanoparticles may be used for treating Alzheimer's disease.

### Example 4. TLR4 and miR-21 Nanoparticle

Toll-like receptor 4 (TLR4) plays a pivotal role in the pathophysiology of spinal cord injury (SCI), particularly in mediating inflammatory responses. TLR4 signaling promotes oxidative stress and apoptosis, leveraging neuronal and glial damage.

miR-21-5p and miR-21-3p are upregulated in SCI to contribute to neuroprotection and axon regeneration. They promote neuronal survival, enhance axonal regrowth, reduce secondary damage, and improve functional recovery. They downregulate PTEN and activate the PI3K/AKT signaling pathway.

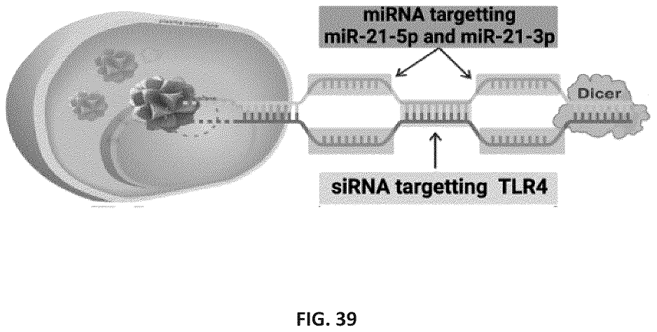

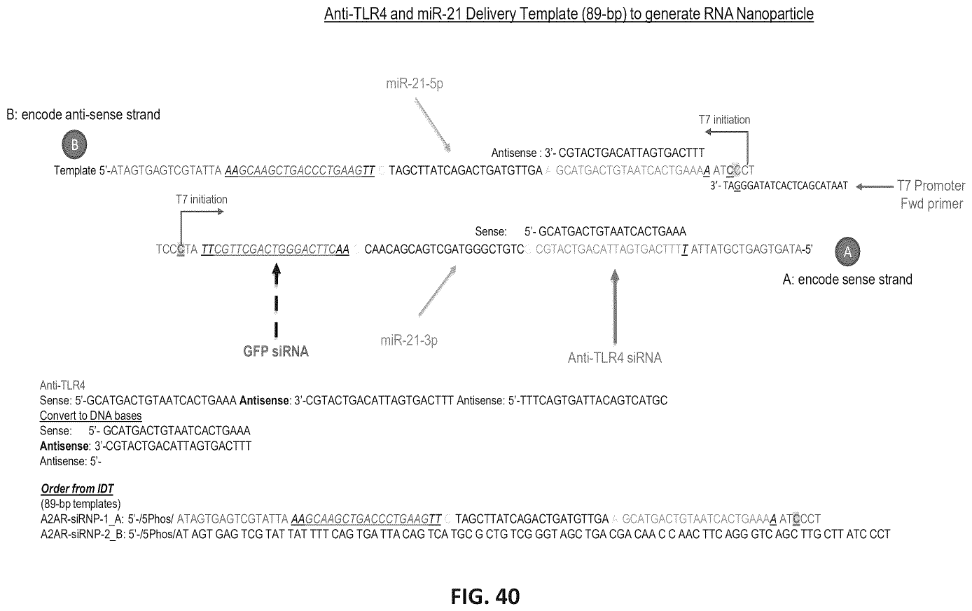

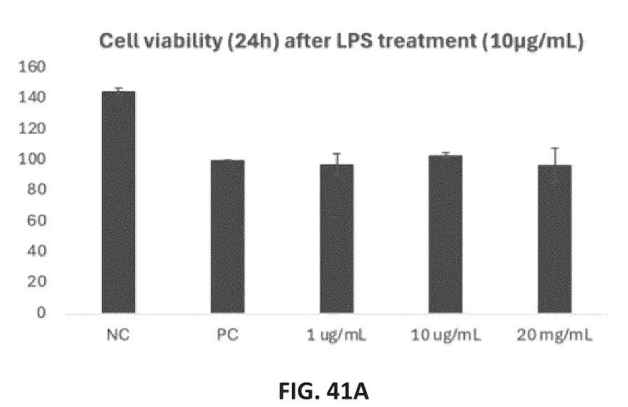

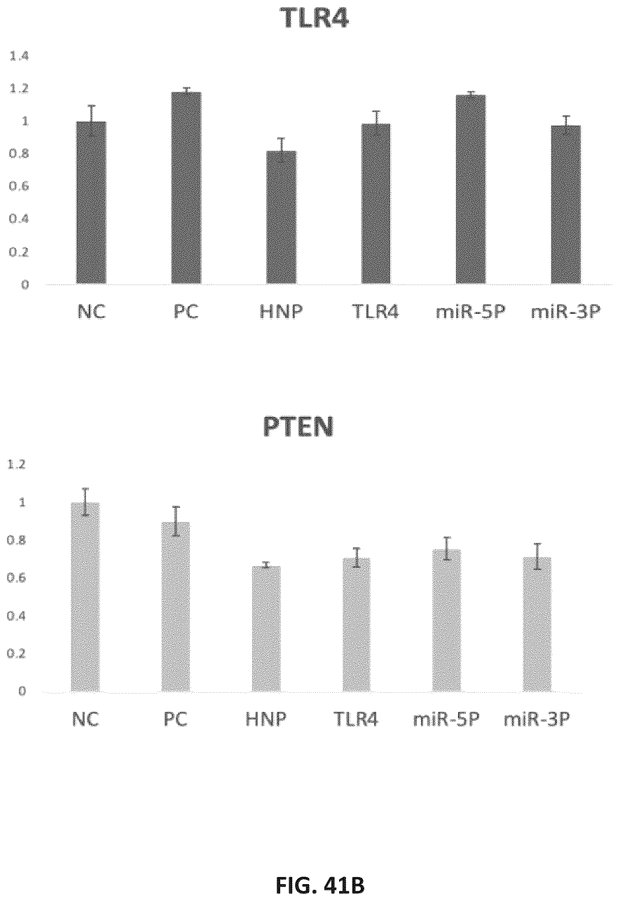

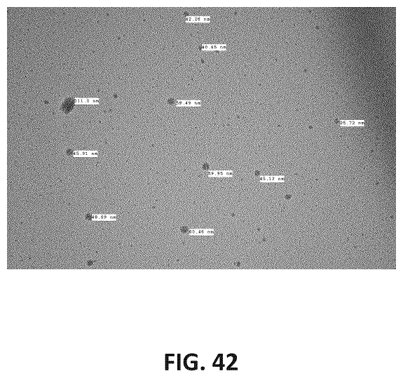

The nanoparticles shown at FIGS. 39-41 are for simultaneous delivery of an siRNA targeting TLR4 and miR-21-5p and miR-21-3p.

### Example 5. Additional Nanoparticles

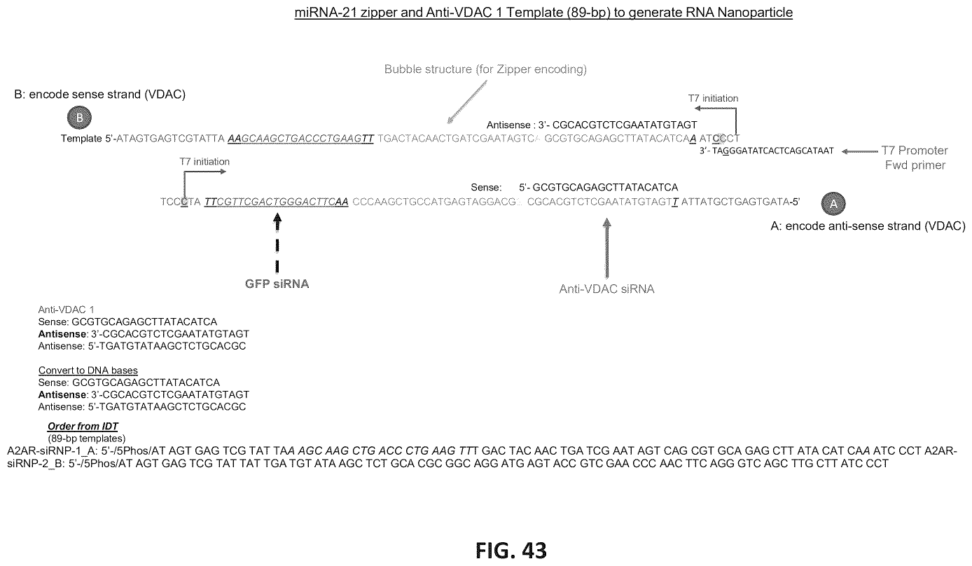

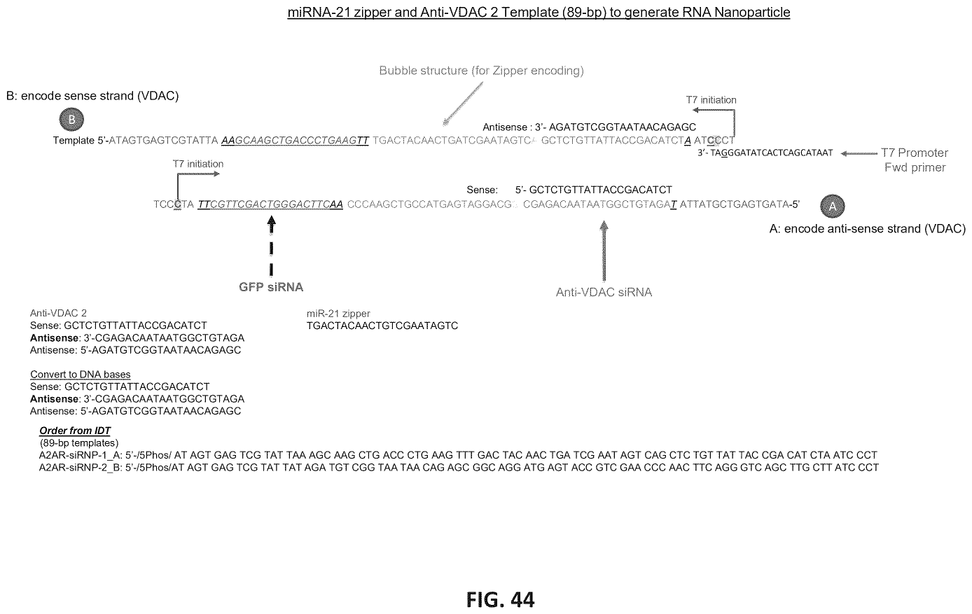

The anti-Glut1 and anti-Ahnak nanoparticles of SEQ ID NOs: 72 and 73 may be used for treating Alzheimer's disease. The nanoparticles shown at FIGS. 43 (miRNA-21 zipper and Anti-VDAC 1) and 44 (miRNA-21 zipper and Anti-VDAC 2) may be used for treatment of breast cancer.

The anti-TNFα and anti-IL-17 nanoparticle of SEQ ID NOs: 70 and 71 may be used for treating psoriasis.

TABLE 2
Informal Sequence Listing

| ID | Description | Sequence (5′-3′) |
|---|---|---|
| 1 | T7 Primer | TAATACGACTCACTATAGGGAT |
| 2 | Template for | /5Phos/ATAGTGAGTCGTATTAAAGCAAGCTGACCCTGAAG |
| | miR-133a- | TCAACCAGCTGCTTTGGTCCCCTGTGGAGCCTATTGCTTCG |
| | Zipper 11G11 | ATTATCCCT |
| | Nanoparticle | |
| | (Bold - GFP | |
| | siRNA | |
| | Underline - | |
| | mi-133a zipper | |
| | Double | |
| | underline - | |
| | spacer G) | |
| 3 | Template for | /5Phos/ATAGTGAGTCGTATTAAATCGAAGCAATAGGCTCCA |
| | complementary | CGTGTCATGAGACGGTCGAACCACCTTCAGGGTCAGCTTGC |
| | sequence of | |
| | 11G11 | TTATCCCT |
| | Nanoparticle | |
| 4 | Template for | /5Phos/ATAGTGAGTCGTATTAAAGCAAGCTGACCCTGAAG |
| | miR-133a- | ACCAGCTGCTTTGGTCCCCTTCAGTGGAGCCTATTGCTTCG |
| | Zipper 14G8 | ATTATCCCT |
| | Nanoparticle | |
| | (GFP siRNA, | |
| | mi-133a | |
| | zipper, spacer | |
| | G) | |
| 5 | Template for | /5Phos/ATAGTGAGTCGTATTAAATCGAAGCAATAGGCTCCA |
| | complementary | CCACGTGTCATGAGACGGTCGAGTCTTCAGGGTCAGCTTGC |
| | sequence of | TTATCCCT |
| | 14G8 | |
| | Nanoparticle | |
| 6 | FAM labeled | /56-FAM/TCCCTATAGTGAGTCGTATT/3AmMO/ |
| | Tag | |
| 7 | Ucp1 forward | ACTGCCACACCTCCAGTCATT |
| | primer | |
| 8 | Ucp1 reverse | CTTTGCCTCACTCAGGATTGG |
| | primer | |
| 9 | Prdm16 | CAGCACGGTGAAGCCATTC |
| | forward primer | |
| 10 | Prdm16 | GCGTGCATCCGCTTGTG |
| | reverse primer | |
| 11 | Fabp4 forward | AAGGTGAAGAGCATCATAACCCT |
| | primer | |
| 12 | Fabp4 reverse | TCACGCCTTTCATAACACATTCC |
| | primer | |
| 13 | Gapdh forward | TGGCCTTCCGTGTTCCTAC |
| | primer | |
| 14 | Gapdh reverse | GAGTTGCTGAAGTCG |
| | primer | |
| 15 | 11A11-zipper | AGGGGACCAAAACAGCTGGTTGA |
| | sequence | |
| 16 | 11G11-zipper | AGGGGACCAAAGCAGCTGGTTGA |
| | sequence | |
| 17 | 14G8-zipper | TGAAGGGGACCAAAGCAGCTGGT |
| | sequence | |
| 18 | miR-133a | TTTGGTCCCCTTCAACCAGCTC |
| | target sequence | |
| 19 | sequence | AAACCAGGGGAAGTTGGTCGAC |
| | complementary | |
| | to miR-133a | |
| | target | |
| 20 | RT primer for | GTCGACCAACTCGCAGAGTTGACTTAACGGCTGAGGTGCT |
| | miR-133a | GTGGTCAACTCTGCG |
| | quantification | |
| 21 | Primer-F for | CTAGTTAGAAGGGGACCAAAGCAGC |
| | miR-133a | |
| | quantification | |
| 22 | Primer-R for | GCTGAGGTGCTGTGGTCA |
| | miR-133a | |
| | quantification | |
| 23 | miR-133a | UUUGGUCCCCUUCAACCAGCUG |
| 24 | miR-133a | TCAACCAGCTGCTTTGGTCCCCT |
| | zipper template | |
| | (11G11) | |
| 25 | miR-133a | ACCAGCTGCTTTGGTCCCCTTCA |
| | zipper template | |
| | (14G8) | |
| 26 | miR-133a | UAAUACGACUCACUAUAGGGAUAAGCAAGCUGACCCUGA |
| | Zipper | AGGUGGUUCGACCGUCUCAUGACACGUGGAGCCUAUUGC |
| | Nanoparticle | UUCGAUUUAAUACGACUCACUAUAGGGAU |
| | 11G11 sense | |
| | strand | |
| 27 | miR-133a | UAAUACGACUCACUAUAGGGAUAAUCGAAGCAAUAGGCU |
| | Zipper | CCACAGGGGACCAAAGCAGCUGGUUGACUUCAGGGUCAG |
| | Nanoparticle | CUUGCUUUAAUACGACUCACUAUAGGGAU |
| | 11G11 | |
| | antisense | |
| | strand | |
| 28 | Template for | /5Phos/ATAGTGAGTCGTATTAAAGCAAGCTGACCCTGAAG |
| | A_{2A}R siRNA | TTCTTAGGCTGGACAACAACCATCTAGCCAGCGAGTCTAAT |
| | Nanoparticle | AACTATAATCCCT |
| | (Bolded - GFP | |
| | siRNA, | |
| | Underlined - | |
| | A_{2A}R siRNA) | |
| 29 | Template for | /5Phos/ATAGTGAGTCGTATTATATAGTTATTAGACTCGCTGG |
| | complementary | CGCTGGATGAAGGACGGTCGAACGCAACTTCAGGGTCAG |
| | sequence of | CTTGCTTATCCCT |
| | A_{2A}R siRNA | |
| | Nanoparticle | |
| 30 | A_{2A}R siRNA | GCCAGCGAGUCUAAUAACUAU |
| | sense | |
| 31 | A_{2A}R siRNA | CGGUCGCUCAGAUUAUUGAUA |
| | antisense | |
| 32 | A_{2A}R siRNA | GCCAGCGAGTCTAATAACTATA |
| | template | |
| 33 | A_{2A}R siRNA | TATAGTTATTAGACTCGCTGGC |
| | complementary | |
| | template | |
| 34 | Template for | /5Phos/ATAGTGAGTCGTATTAAAGCAAGCTGACCCTGAAG |
| | PDE2A2 | TTCTTAGGCTGGACAACAACCATCTAGCCTGAGGACGACA |
| | siRNA | CTTCTATAATCCCT |
| | Nanoparticle | |
| | (Bolded - GFP | |
| | SIRNA, | |
| | Underlined - | |
| | PDE2A | |
| | siRNA) | |
| 35 | Template for | /5Phos/ATAGTGAGTCGTATTATATAGAAGTGTCGTCCTCAG |
| | complementary | GCGCTGGATGAAGGACGGTCGAACGCAACTTCAGGGTCAG |
| | sequence of | CTTGCTTATCCCT |
| | PDE2A2 | |
| | siRNA | |
| | Nanoparticle | |
| 36 | PDE2A2 | GCCUGAGGACGACACUUCUAU |
| | siRNA sense | |
| 37 | PDE2A2 | AUAGAAGUGUCGUCCUCAGGC |
| | siRNA | |
| | antisense | |
| 38 | PDE2A2 | GCCTGAGGACGACACTTCTAT |
| | siRNA | |
| | template | |
| 39 | PDE2A2 | ATAGAAGTGTCGTCCTCAGGC |
| | siRNA | |
| | complementary | |
| | template | |
| 40 | Template for | /5Phos/ATAGTGAGTCGTATTAAAGCAAGCTGACCCTGAAG |
| | truncated | TTCTTAGGCTGGACAACAACCATCTAGTCTGAGTAGGTGTC |
| | PDE2A siRNA | ATTCTAAATCCCT |
| | Nanoparticle | |
| | (Bolded - | |
| | GFP siRNA, | |
| | Underlined - | |
| | PDE2A | |
| | siRNA) | |
| 41 | Template for | /5Phos/ATAGTGAGTCGTATTATTAGAATGACTCCTACTCAG |
| | complementary | ACGCTGGATGAAGGACGGTCGAACGCAACTTCAGGGTCAG |
| | sequence of | CTTGCTTATCCCT |
| | truncated | |
| | PDE2A | |
| | siRNA | |
| | Nanoparticle | |
| 42 | PDE2A siRNA | GTCTGAGTAGGTGTCATTCTA |
| | template | |
| 43 | PDE2A siRNA | TAGAATGACTCCTACTCAGAC |
| | complementary | |
| | template | |
| 44 | PDE2A siRNA | GUCUGAGUAGGUGUCAUUCUA |
| | sense | |
| 45 | PDE2A siRNA | UAGAAUGACUCCUACUCAGAC |
| | antisense | |
| 46 | Template for | /5Phos/ATAGTGAGTCGTATTAAAGCAAGCTGACCCTGAAG |
| | TLR4 siRNA | TTCTAGCTTATCAGACTGATGTTGAAGCATGACTGTAATCACT |
| | and miR-21 | GAAAAATCCCT |
| | Nanoparticle | |
| | (Bolded - GFP | |
| | SIRNA, | |
| | Underlined - | |
| | TLR4 siRNA, | |
| | Italicized - | |
| | miR-21-5p) | |
| 47 | Template for | /5Phos/ATAGTGAGTCGTATTATTTTCAGTGATTACAGTCATG |
| | complementary | CGCTGTCGGGTAGCTGACGACAACCAACTTCAGGGTCAGCT |
| | sequence of | TGCTTATCCCT |
| | TLR4 siRNA | |
| | and miR-21 | |
| | Nanoparticle | |
| | (Bolded - GFP | |
| | siRNA, | |
| | Underlined - | |
| | TLR4 siRNA, | |
| | Italicized - | |
| | miR-21-3p) | |
| 48 | TLR4 siRNA | GCATGACTGTAATCACTGAAA |
| | template | |
| 49 | TLR4 siRNA | TTTCAGTGATTACAGTCATG |
| | complementary | |
| | template | |
| 50 | TLR4 siRNA | GCAUGACUGUAAUCACUGAAA |
| | sense | |
| 51 | TLR4 siRNA | UUUCAGUGAUUACAGUCAUGC |
| | antisense | |
| 52 | miR-21-3p | CTGTCGGGTAGCTGACGACAAC |
| | Template | |
| 53 | miR-21-5p | TAGCTTATCAGACTGATGTTGA |
| | Template | |
| 54 | miR-21-3p | CAACAGCAGUCGAUGGGCUGUC |
| 55 | miR-21-5p | UAGCUUAUCAGACUGAUGUUGA |
| 56 | Template for | /5Phos/ATAGTGAGTCGTATTAAAGCAAGCTGACCCTGAAGT |
| | miR-21 zipper | TTGACTACAACTGATCGAATAGTCAGCGTGCAGAGCTTATACA |
| | and anti- | TCAAATCCCT |
| | VDAC 1 | |
| | Nanoparticle | |
| | (Bolded - GFP | |
| | SIRNA, | |
| | Underlined - | |
| | VDAC 1 | |
| | siRNA, | |
| | Italicized - | |
| | miRNA-21 | |
| | zipper) | |
| 57 | Template for | /5Phos/ATAGTGAGTCGTATTATTGATGTATAAGCTCTGCAC |
| | complementary | GCGGCAGGATGAGTACCGTCGAACCCAACTTCAGGGTCAG |
| | sequence of | CTTGCTTATCCCT |
| | miR-21 zipper | |
| | and anti- | |
| | VDAC 1 | |
| | Nanoparticle | |
| 58 | VDAC 1 | GCGTGCAGAGCTTATACATCA |
| | siRNA | |
| | template | |
| 59 | VDAC 1 | TGATGTATAAGCTCTGCACGC |
| | siRNA | |
| | complementary | |
| | template | |
| 60 | VDAC 1 | GCGUGCAGAGCUUAUACAUCA |
| | siRNA sense | |
| 61 | VDAC 1 | UGAUGUAUAAGCUCUGCACGC |
| | siRNA | |
| | antisense | |
| 62 | miR-21 zipper | TGACTACAACTGATCGAATAGTC |
| | template | |
| 63 | Template for | /5Phos/ATAGTGAGTCGTATTAAAGCAAGCTGACCCTGAAG |
| | miR-21 zipper | TTTGACTACAACTGATCGAATAGTCAGCTCTGTTATTACCGAC |
| | and anti- | ATCTAATCCCT |
| | VDAC 2 | |
| | Nanoparticle | |
| | (Bolded - GFP | |
| | SIRNA, | |
| | Underlined - | |
| | VDAC 2 | |
| | SIRNA, | |
| | Italicized - | |
| | miRNA-21 | |
| | zipper) | |
| 64 | Template for | /5Phos/ATAGTGAGTCGTATTATAGATGTCGGTAATAACAGA |
| | complementary | GCGGCAGGATGAGTACCGTCGAACCCAACTTCAGGGTCAG |
| | sequence of | CTTGCTTATCCCT |
| | miR-21 zipper | |
| | and anti- | |
| | VDAC 2 | |
| | Nanoparticle | |
| 65 | VDAC 2 | GCTCTGTTATTACCGACATCT |
| | siRNA | |
| | template | |
| 66 | VDAC 2 | AGATGTCGGTAATAACAGAGC |
| | siRNA | |
| | complementary | |
| | template | |
| 67 | VDAC 2 | GCUCUGUUAUUACCGACAUCU |
| | siRNA sense | |
| 68 | VDAC 2 | AGAUGUCGGUAAUAACAGAGC |
| | siRNA | |
| | antisense | |
| 69 | miR-21 zipper | TGACTACAACTGTCGAATAGTC |
| | template | |
| 70 | Template for | /5Phos/ATAGTGAGTCGTATTAAAGCAAGCTGACCCTGAAGTT |
| | anti-TNFa and | CTTAGGCTGGACAACAACCATCTAGCCAGCGAGTCTAATAA |
| | anti-IL-17 | CTATAATCCCT |
| | Nanoparticle | |
| 71 | Template for | /5Phos/ATAGTGAGTCGTATTATATAGTTATTAGACTCGCTGG |
| | complementary | CGCTGGATGAAGGACGGTCGAACGCAACTTCAGGGTCAGCT |
| | sequence of | TGCTTATCCCT |
| | anti- TNFa and | |
| | anti-IL-17 | |
| | Nanoparticle | |
| 72 | Template for | /5Phos/ATAGTGAGTCGTATTAAAGCAAGCTGACCCTGAAGTT |
| | anti-GLUT1 | CTTAGGCTGGACAACAACCATCTAGCTACTGACTGGATAAG |
| | and anti-Ahnak | ATCTATCCCT |
| | Nanoparticle | |
| 73 | Template for | /5Phos/ATAGTGAGTCGTATTAAGATCTTATCCAGTCAGTAGC |
| | complementary | GCTGGATGAAGGACGGTCGAACGCAACTTCAGGGTCAGCTT |
| | sequence of | GCTTATCCCTs |
| | anti- GLUT1 | |
| | and anti- | |
| | Ahnak | |
| | Nanoparticle | |
| 74 | General | /5Phos/ATAGTGAGTCGTATTATN1(21-23)GN2(21-23)CN3(21- |
| | Template | 23)ATCCCT |
| 75 | miR-21 zipper | UGACUACAACUGUCGAAUAGUC |
| | template | |
| 76 | Ahnak siRNP- | /5Phos/ATAGTGAGTCGTATTAAAGCAAGCTGACCCTGAAGTT |
| | 1_A human | CTTAGGCTGGACAACAACCATCTAGGTGGAGCCTTGACAAC |
| | | GTAGTATCCCT |
| 77 | Ahnak siRNP- | /5Phos/ATAGTGAGTCGTATTATCACCTCGGAACTGTTGCATC |
| | 1_B human | AGCTGGATGAAGGACGGTCGAACGCAACTTCAGGGTCAGC |
| | | TTGCTTATCCCT |
| 78 | Ahnak | /5Phos/ATAGTGAGTCGTATTAAAGCAAGCTGACCCTGAAGTT |
| | siRNP_mouse- | CTTAGGCTGGACAACAACCATCTAGGAGCCGGTGACTAATT |
| | 1_A | CAAAATCCCT |
| 79 | Ahnak | /5Phos/ATAGTGAGTCGTATTATTTGAATTAGTCACCGGCTCC |
| | siRNP_mouse- | GCTGGATGAAGGACGGTCGAACGCAACTTCAGGGTCAGCTT |
| | 1_B | GCTTATCCCT |

We claim:

1. A composition comprising:
a first DNA template comprising from 5′ to 3′:
a first portion of a T7 promoter;
a first double-strand template;
a first single-strand template;
a second double-strand template; and
a second portion of a T7 promoter; and
a second DNA template comprising from 5′ to 3′:
the first portion of the T7 promoter;
a second complement template that is reverse complementary to the second double-strand template;
a second single-strand template;
a first complement template that is reverse complementary to the first double-strand template; and
the second portion of the T7 promoter;
wherein the first single-strand template and the second single-strand template cannot hybridize;
wherein the first double-strand template and the first complement template encode a first siRNA or a first random duplex; and
wherein the second double-strand template and the second complement template encode a second siRNA or a second random duplex.

2. The composition of claim 1, wherein the first DNA template and the second DNA template are between about 85 and about 95 nucleotides in length.

3. The composition of claim 2, wherein the first DNA template and the second DNA template each are about 90 nucleotides in length.

4. The composition of claim 1, wherein each of the first single-strand template and the second single-strand template are between about 21 and about 23 nucleotides in length.

5. The composition of claim 1, wherein each of the first single-strand template and the second single-strand template encodes a miRNA, a miRNA zipper, a miRNA mimic, a miRNA inhibitor, an antagomir, an aptamer, an antisense oligonucleotide (ASO), or a random sequence.

6. The composition of claim 1, wherein the first siRNA or random duplex and the second siRNA or random duplex each are between about 21 and about 23 base pairs in length.

7. The composition of claim 1, wherein the first siRNA or random duplex and the second siRNA or random duplex are different.

8. A kit comprising the first DNA template and the second DNA template of claim 1, wherein the first DNA template and the second DNA template are in separate containers.

9. An inhibitory RNA nanoparticle comprising a first RNA and a second RNA, wherein the first and second RNAs are partially hybridized to each other;
the nanoparticle comprising from 5′ to 3′:
a first double-stranded region, wherein the first RNA is hybridized to the second RNA;
a bubble region, wherein the first RNA is not hybridized to the second RNA; and
a second double-stranded region, wherein the first RNA is hybridized to the second RNA;
wherein the first RNA is hybridized to the second RNA at the first double-stranded region and the second double-stranded region;
wherein the first RNA and the second RNA cannot hybridize to each other; and
wherein the first and second RNAs are between about 85 and about 95 nucleotides in length.

10. The nanoparticle of claim 9, wherein the first RNA and the second RNA are each about 90 nucleotides in length.

11. The nanoparticle of claim 9, wherein in the bubble region, the first RNA and the second RNA are between about 21 and about 23 nucleotides in length; and wherein the first double-stranded region and the second double-stranded region are each between about 21 and about 23 base pairs in length.

12. The nanoparticle of claim 9, wherein in the bubble region, each of the first RNA and the second RNA is a miRNA, a miRNA zipper, a miRNA mimic, a miRNA inhibitor, an antagomir, an aptamer, an ASO, or a random sequence.

13. The nanoparticle of claim 9, wherein the first double-stranded region and the second double-stranded region are different.

14. The nanoparticle of claim 9, wherein each of the first double-stranded region and the second double-stranded region is an siRNA or a random duplex.

15. The nanoparticle of claim 9, wherein the nanoparticle comprises a fluorescent label.

16. The nanoparticle of claim 15, wherein the fluorescent label comprises 6-carboxyfluorescein (FAM).

17. The nanoparticle of claim 9, wherein the nanoparticle is coated with a polymer or a lipid.

18. A method for preparing the inhibitory RNA nanoparticle of claim 11, the method comprising:
providing a first 5′ phosphorylated linear DNA template encoding the first RNA and a second 5′ phosphorylated linear DNA template encoding the second RNA; and for each template:
a) in a reaction mixture, heating the template with a primer DNA containing a T7 promoter sequence in a duplex buffer at about 95° C. for about 2 minutes;
b) letting the reaction mixture cool to room temperature;
c) adding T4 buffer comprising 10 mM MgCl2, about 2.5 mM MnCl2, and about 200 U μL⁻¹ T4 DNA ligase;
d) incubating the reaction mixture for about 16° C. for about 18 hours;
e) incubating the reaction mixture for about 65° C. for about 10 minutes;
f) diluting the reaction mixture to about 1:10 produce a first circularized DNA template and a second circularized DNA template;
g) adding EDTA to the reaction mixture to about 11 mM, and incubating the reaction at about 80° C. for about 30 minutes;
h) adding an equimolar amount of the first and second circularized DNA template to a reaction mixture comprising T7 reaction buffer, ribonucleotides, and a T7 RNA polymerase;
i) incubating the reaction mixture at about 37° C. for about 20 hours, thereby producing the inhibitory RNA nanoparticle; and
j) washing the inhibitory RNA nanoparticle.

19. The method of claim 18, further comprising:
k) incubating the inhibitory RNA nanoparticle with a fluorescently labeled oligonucleotide comprising a sequence complementary to a non-functional portion of the inhibitory RNA nanoparticle at a final concentration of about 10 μM at about 65° C. for about 10 minutes.

20. The method of claim 18, wherein step f) further comprises incubating the reacting mixture with at least one of exonuclease 1 and exonuclease 3 at about 37° C. for about 30 minutes.


SEQUENCE LISTING (85 sequences)

SEQ ID NO: 1 - DNA, 22 bp, synthetic construct
  source 1..22: mol_type = other DNA
taatacgactcactatagggat

SEQ ID NO: 2 - DNA, 85 bp, synthetic construct
  source 1..85: mol_type = other DNA
atagtgagtcgtattaaagcaagctgaccctgaagtcaaccagctgctttggtcccctgtggagcctattgcttcgattatccct

SEQ ID NO: 3 - DNA, 85 bp, synthetic construct
  source 1..85: mol_type = other DNA
atagtgagtcgtattaaatcgaagcaataggctccacgtgtcatgagacggtcgaaccaccttcagggtcagcttgcttatccct

SEQ ID NO: 4 - DNA, 85 bp, synthetic construct
  source 1..85: mol_type = other DNA
atagtgagtcgtattaaagcaagctgaccctgaagaccagctgctttggtccccttcagtggagcctattgcttcgattatccct

SEQ ID NO: 5 - DNA, 85 bp, synthetic construct
  source 1..85: mol_type = other DNA
atagtgagtcgtattaaatcgaagcaataggctccaccacgtgtcatgagacggtcgagtcttcagggtcagcttgcttatccct

SEQ ID NO: 6 - DNA, 20 bp, synthetic construct
  source 1..20: mol_type = other DNA
tccctatagtgagtcgtatt

SEQ ID NO: 7 - DNA, 21 bp, synthetic construct
  source 1..21: mol_type = other DNA
actgccacacctccagtcatt

SEQ ID NO: 8 - DNA, 21 bp, synthetic construct
  source 1..21: mol_type = other DNA
ctttgcctcactcaggattgg

SEQ ID NO: 9 - DNA, 19 bp, synthetic construct
  source 1..19: mol_type = other DNA
cagcacggtgaagccattc

SEQ ID NO: 10 - DNA, 17 bp, synthetic construct
  source 1..17: mol_type = other DNA
gcgtgcatccgcttgtg

SEQ ID NO: 11 - DNA, 23 bp, synthetic construct
  source 1..23: mol_type = other DNA
aaggtgaagagcatcataaccct

SEQ ID NO: 12 - DNA, 23 bp, synthetic construct
  source 1..23: mol_type = other DNA
tcacgcctttcataacacattcc

SEQ ID NO: 13 - DNA, 19 bp, synthetic construct
  source 1..19: mol_type = other DNA
tggccttccgtgttcctac

SEQ ID NO: 14 - DNA, 15 bp, synthetic construct
  source 1..15: mol_type = other DNA
gagttgctgaagtcg

SEQ ID NO: 15 - DNA, 23 bp, synthetic construct
  source 1..23: mol_type = other DNA
aggggaccaaaacagctggttga

SEQ ID NO: 16 - DNA, 23 bp, synthetic construct
  source 1..23: mol_type = other DNA
aggggaccaaagcagctggttga

SEQ ID NO: 17 - DNA, 23 bp, synthetic construct
  source 1..23: mol_type = other DNA
tgaaggggaccaaagcagctggt

SEQ ID NO: 18 - DNA, 22 bp, synthetic construct
  source 1..22: mol_type = other DNA
tttggtccccttcaaccagctc

SEQ ID NO: 19 - DNA, 22 bp, synthetic construct
  source 1..22: mol_type = other DNA
aaaccaggggaagttggtcgac

SEQ ID NO: 20 - DNA, 55 bp, synthetic construct
  source 1..55: mol_type = other DNA
gtcgaccaactcgcagagttgacttaacggctgaggtgctgtggtcaactctgcg

SEQ ID NO: 21 - DNA, 25 bp, synthetic construct
  source 1..25: mol_type = other DNA
ctagttagaaggggaccaaagcagc

SEQ ID NO: 22 - DNA, 18 bp, synthetic construct
  source 1..18: mol_type = other DNA
gctgaggtgctgtggtca

SEQ ID NO: 23 - RNA, 22 nt, synthetic construct
  source 1..22: mol_type = other RNA
tttggtccccttcaaccagctg

SEQ ID NO: 24 - DNA, 23 bp, synthetic construct
  source 1..23: mol_type = other DNA
tcaaccagctgctttggtcccct

SEQ ID NO: 25 - DNA, 23 bp, synthetic construct
  source 1..23: mol_type = other DNA
accagctgctttggtccccttca

SEQ ID NO: 26 - RNA, 107 nt, synthetic construct
  source 1..107: mol_type = other RNA
taatacgactcactatagggataagcaagctgaccctgaaggtggttcgaccgtctcatgacacgtggagcctattgcttcgatttaatacgactcactatagggat

SEQ ID NO: 27 - RNA, 107 nt, synthetic construct
  source 1..107: mol_type = other RNA
taatacgactcactatagggataatcgaagcaataggctccacaggggaccaaagcagctggttgacttcagggtcagcttgctttaatacgactcactatagggat

SEQ ID NO: 28 - DNA, 89 bp, synthetic construct
  source 1..89: mol_type = other DNA
atagtgagtcgtattaaagcaagctgaccctgaagttcttaggctggacaacaaccatctagccagcgagtctaataactataatccct

SEQ ID NO: 29 - DNA, 89 bp, synthetic construct
  source 1..89: mol_type = other DNA
atagtgagtcgtattatatagttattagactcgctggcgctggatgaaggacggtcgaacgcaacttcagggtcagcttgcttatccct

SEQ ID NO: 30 - RNA, 21 nt, synthetic construct
  source 1..21: mol_type = other RNA
gccagcgagtctaataactat

SEQ ID NO: 31 - RNA, 21 nt, synthetic construct
  source 1..21: mol_type = other RNA
cggtcgctcagattattgata

SEQ ID NO: 32 - DNA, 22 bp, synthetic construct
  source 1..22: mol_type = other DNA
gccagcgagtctaataactata

SEQ ID NO: 33 - DNA, 22 bp, synthetic construct
  source 1..22: mol_type = other DNA
tatagttattagactcgctggc

SEQ ID NO: 34 - DNA, 89 bp, synthetic construct
  source 1..89: mol_type = other DNA
atagtgagtcgtattaaagcaagctgaccctgaagttcttaggctggacaacaaccatctagcctgaggacgacacttctataatccct

SEQ ID NO: 35 - DNA, 89 bp, synthetic construct
  source 1..89: mol_type = other DNA
atagtgagtcgtattatatagaagtgtcgtcctcaggcgctggatgaaggacggtcgaacgcaacttcagggtcagcttgcttatccct

SEQ ID NO: 36 - RNA, 21 nt, synthetic construct
  source 1..21: mol_type = other RNA
gcctgaggacgacacttctat

SEQ ID NO: 37 - RNA, 21 nt, synthetic construct
  source 1..21: mol_type = other RNA
atagaagtgtcgtcctcaggc

SEQ ID NO: 38 - DNA, 21 bp, synthetic construct
  source 1..21: mol_type = other DNA
gcctgaggacgacacttctat

SEQ ID NO: 39 - DNA, 21 bp, synthetic construct
  source 1..21: mol_type = other DNA
atagaagtgtcgtcctcaggc

SEQ ID NO: 40 - DNA, 89 bp, synthetic construct
  source 1..89: mol_type = other DNA
atagtgagtcgtattaaagcaagctgaccctgaagttcttaggctggacaacaaccatctagtctgagtaggtgtcattctaaatccct

SEQ ID NO: 41 - DNA, 89 bp, synthetic construct
  source 1..89: mol_type = other DNA
atagtgagtcgtattattagaatgactcctactcagacgctggatgaaggacggtcgaacgcaacttcagggtcagcttgcttatccct

SEQ ID NO: 42 - DNA, 21 bp, synthetic construct
  source 1..21: mol_type = other DNA
gtctgagtaggtgtcattcta

SEQ ID NO: 43 - DNA, 21 bp, synthetic construct
  source 1..21: mol_type = other DNA
tagaatgactcctactcagac

SEQ ID NO: 44 - RNA, 21 nt, synthetic construct
  source 1..21: mol_type = other RNA
gtctgagtaggtgtcattcta

SEQ ID NO: 45 - RNA, 21 nt, synthetic construct
  source 1..21: mol_type = other RNA
tagaatgactcctactcagac

SEQ ID NO: 46 - DNA, 89 bp, synthetic construct
  source 1..89: mol_type = other DNA
atagtgagtcgtattaaagcaagctgaccctgaagttctagcttatcagactgatgttgaagcatgactgtaatcactgaaaaatccct

SEQ ID NO: 47 - DNA, 89 bp, synthetic construct
  source 1..89: mol_type = other DNA
atagtgagtcgtattattttcagtgattacagtcatgcgctgtcgggtagctgacgacaaccaacttcagggtcagcttgcttatccct

SEQ ID NO: 48 - DNA, 21 bp, synthetic construct
  source 1..21: mol_type = other DNA
gcatgactgtaatcactgaaa

SEQ ID NO: 49 - DNA, 20 bp, synthetic construct
  source 1..20: mol_type = other DNA
tttcagtgattacagtcatg

SEQ ID NO: 50 - RNA, 21 nt, synthetic construct
  source 1..21: mol_type = other RNA
gcatgactgtaatcactgaaa

SEQ ID NO: 51 - RNA, 21 nt, synthetic construct
  source 1..21: mol_type = other RNA
tttcagtgattacagtcatgc

SEQ ID NO: 52 - DNA, 22 bp, synthetic construct
  source 1..22: mol_type = other DNA
ctgtcgggtagctgacgacaac

SEQ ID NO: 53 - DNA, 22 bp, synthetic construct
  source 1..22: mol_type = other DNA
tagcttatcagactgatgttga

SEQ ID NO: 54 - RNA, 22 nt, synthetic construct
  source 1..22: mol_type = other RNA
caacagcagtcgatgggctgtc

SEQ ID NO: 55 - RNA, 22 nt, synthetic construct
  source 1..22: mol_type = other RNA
tagcttatcagactgatgttga

SEQ ID NO: 56 - DNA, 89 bp, synthetic construct
  source 1..89: mol_type = other DNA
atagtgagtcgtattaaagcaagctgaccctgaagtttgactacaactgatcgaatagtcagcgtgcagagcttatacatcaaatccct

SEQ ID NO: 57 - DNA, 89 bp, synthetic construct
  source 1..89: mol_type = other DNA
atagtgagtcgtattattgatgtataagctctgcacgcggcaggatgagtaccgtcgaacccaacttcagggtcagcttgcttatccct

SEQ ID NO: 58 - DNA, 21 bp, synthetic construct
  source 1..21: mol_type = other DNA
gcgtgcagagcttatacatca

SEQ ID NO: 59 - DNA, 21 bp, synthetic construct
  source 1..21: mol_type = other DNA
tgatgtataagctctgcacgc

SEQ ID NO: 60 - RNA, 21 nt, synthetic construct
  source 1..21: mol_type = other RNA
gcgtgcagagcttatacatca

SEQ ID NO: 61 - RNA, 21 nt, synthetic construct
  source 1..21: mol_type = other RNA
tgatgtataagctctgcacgc

SEQ ID NO: 62 - DNA, 23 bp, synthetic construct
  source 1..23: mol_type = other DNA
tgactacaactgatcgaatagtc

SEQ ID NO: 63 - DNA, 89 bp, synthetic construct
  source 1..89: mol_type = other DNA
atagtgagtcgtattaaagcaagctgaccctgaagtttgactacaactgatcgaatagtcagctctgttattaccgacatctaatccct

SEQ ID NO: 64 - DNA, 89 bp, synthetic construct
  source 1..89: mol_type = other DNA
atagtgagtcgtattatagatgtcggtaataacagagcggcaggatgagtaccgtcgaacccaacttcagggtcagcttgcttatccct

SEQ ID NO: 65 - DNA, 21 bp, synthetic construct
  source 1..21: mol_type = other DNA
gctctgttattaccgacatct

SEQ ID NO: 66 - DNA, 21 bp, synthetic construct
  source 1..21: mol_type = other DNA
agatgtcggtaataacagagc

SEQ ID NO: 67 - RNA, 21 nt, synthetic construct
  source 1..21: mol_type = other RNA
gctctgttattaccgacatct

SEQ ID NO: 68 - RNA, 21 nt, synthetic construct
  source 1..21: mol_type = other RNA
agatgtcggtaataacagagc

SEQ ID NO: 69 - DNA, 22 bp, synthetic construct
  source 1..22: mol_type = other DNA
tgactacaactgtcgaatagtc

SEQ ID NO: 70 - DNA, 89 bp, synthetic construct; identical to SEQ ID NO: 28
  source 1..89: mol_type = other DNA

SEQ ID NO: 71 - DNA, 89 bp, synthetic construct; identical to SEQ ID NO: 29
  source 1..89: mol_type = other DNA

SEQ ID NO: 72 - DNA, 88 bp, synthetic construct
  source 1..88: mol_type = other DNA
atagtgagtcgtattaaagcaagctgaccctgaagttcttaggctggacaacaaccatctagctactgactggataagatctatccct

SEQ ID NO: 73 - DNA, 88 bp, synthetic construct
  source 1..88: mol_type = other DNA
atagtgagtcgtattaagatcttatccagtcagtagcgctggatgaaggacggtcgaacgcaacttcagggtcagcttgcttatccct

SEQ ID NO: 74 - DNA, 28 bp, synthetic construct
  source 1..28: mol_type = other DNA
  variation 18: note = n= 21-23
  variation 20: note = n=21-23
  variation 22: note = n=21-23
atagtgagtcgtattatngncnatccct

SEQ ID NO: 75 - RNA, 22 nt, synthetic construct
  source 1..22: mol_type = other RNA
tgactacaactgtcgaatagtc

SEQ ID NO: 76 - DNA, 89 bp, synthetic construct
  source 1..89: mol_type = other DNA
atagtgagtcgtattaaagcaagctgaccctgaagttcttaggctggacaacaaccatctaggtggagccttgacaacgtagtatccct

SEQ ID NO: 77 - DNA, 89 bp, synthetic construct
  source 1..89: mol_type = other DNA
atagtgagtcgtattatcacctcggaactgttgcatcagctggatgaaggacggtcgaacgcaacttcagggtcagcttgcttatccct

SEQ ID NO: 78 - DNA, 88 bp, synthetic construct
  source 1..88: mol_type = other DNA
atagtgagtcgtattaaagcaagctgaccctgaagttcttaggctggacaacaaccatctaggagccggtgactaattcaaaatccct

SEQ ID NO: 79 - DNA, 88 bp, synthetic construct
  source 1..88: mol_type = other DNA
atagtgagtcgtattatttgaattagtcaccggctccgctggatgaaggacggtcgaacgcaacttcagggtcagcttgcttatccct

SEQ ID NO: 80 - DNA, 67 bp, synthetic construct
  source 1..67: mol_type = other DNA
tcccctggtttcgtcgaccaactcgcagagttgacttaacggctgaggtgctgtggtcaactctgcg

SEQ ID NO: 81 - DNA, 68 bp, synthetic construct
  source 1..68: mol_type = other DNA
ctagttagaaggggaccaaagcagctggttgagcgtctcaactgaattgccgactccacgacaccagt

SEQ ID NO: 82 - RNA, 21 nt, synthetic construct
  source 1..21: mol_type = other RNA
ggaccaaactcccgttagtca

SEQ ID NO: 83 - RNA, 21 nt, synthetic construct
  source 1..21: mol_type = other RNA
tgactaacgggagtttggtcc

SEQ ID NO: 84 - DNA, 21 bp, synthetic construct
  source 1..21: mol_type = other DNA
ggaccaaactcccgttagtca

SEQ ID NO: 85 - DNA, 21 bp, synthetic construct
  source 1..21: mol_type = other DNA
tgactaacgggagtttggtcc